(19) United States
(12) Patent Application Publication
(10) Pub. No.: US 2022/0132847 A1
(43) Pub. Date: May 5, 2022

(54) CINNOLINIUM COMPOUNDS FOR USE IN A METHOD OF CONTROLLING UNWANTED PLANT GROWTH

(71) Applicant: SYNGENTA CROP PROTECTION AG, Basel (CH)

(72) Inventors: James Nicholas SCUTT, Bracknell, Berkshire (GB); Nigel James WILLETTS, Bracknell, Berkshire (GB)

(73) Assignee: SYNGENTA CROP PROTECTION AG, Basel (CH)

(21) Appl. No.: 17/429,193

(22) PCT Filed: Feb. 4, 2020
(86) PCT No.: PCT/EP2020/052743

§ 371 Date: Aug. 6, 2021

(30) Foreign Application Priority Data
Feb. 11, 2019 (GB) 1901808.4

(51) Int. Cl. A01N 43/58; A01P 13/00; C07F 9/30; A01N 57/22; C07D 237/28; C07F 9/38

(52) U.S. Cl. CPC A01N 43/58; A01P 13/00; C07F 9/38; A01N 57/22; C07D 237/28; C07F 9/30

(57) ABSTRACT

The present invention relates to herbicidally active cinnolinium derivatives of formula (I), as well as to processes and intermediates used for the preparation of such derivatives. The invention further extends to herbicidal compositions comprising such derivatives, as well as to the use of such compounds and compositions in controlling undesirable plant growth: in particular the use in controlling weeds, in crops of useful plants.

The present invention relates to herbicidally active cinnolinium derivatives, as well as to processes and intermediates used for the preparation of such derivatives. The invention further extends to herbicidal compositions comprising such derivatives, as well as to the use of such compounds and compositions in controlling undesirable plant growth: in particular, the use in controlling weeds, in crops of useful plants.

U.S. Pat. No. 4,666,499 describes a selection of 2-methyl-4-phosphinyl cinnolinium hydroxide salts and their use as herbicides. Gardner et al., (J Agric Food Chem. 1992, 40:318-321) investigate the herbicidal mode of action of 2-methylcinnolinium-4-(O-methyl phosphonate).

The present invention is based on the finding that cinnolinium derivatives of formula (I) as defined herein exhibit surprising good herbicidal activity and are particularly useful in non-selective burn-down applications.

Thus, in a first aspect, the invention provides a compound of formula (I) or an agronomically acceptable salt or zwitterionic species thereof:

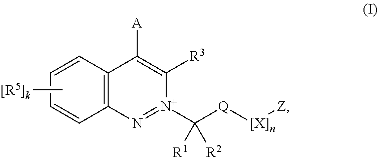

wherein

R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;

R² is selected from the group consisting of hydrogen, halogen, C₁-C₆alkyl and C₁-C₆haloalkyl; and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl;

or R¹ and R² together with the carbon atom to which they are attached form a C₃-C₆cycloalkyl ring or a 3- to 6-membered heterocyclyl, which comprises 1 or 2 heteroatoms individually selected from N and O;

Q is (CR^{1a}R^{2b})ₘ;

m is an integer of 0, 1, 2 or 3;

each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O;

R³ is selected from the group consisting of hydrogen, halogen, cyano, nitro, —S(O)ᵣR¹⁵, C₁-C₆alkyl, C₁-C₆haloalkyl, C₁-C₆haloalkoxy, C₁-C₆alkoxy, C₃-C₆cycloalkyl, —N(R⁶)₂, phenyl, a 5- or 6-membered heteroaryl comprising 1, 2, 3 or 4 heteroatoms individually selected from N, O and S, and a 4- to 6-membered heterocyclyl comprising 1, 2 or 3 heteroatoms individually selected from N, O and S, and wherein said phenyl, heteroaryl or heterocyclyl moieties are optionally substituted by 1 or 2 R⁹ substituents;

A is selected from the group consisting of —C(O)OR⁴¹⁰, —CHO, —C(O)R⁴²⁴, —C(O)NHOR⁴¹¹, —C(O)NHCN, —C(O)NHR⁴²⁵, —S(O)₂NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶(CR⁴⁶₂)_{q}—S(O)₂OR⁴¹⁰, —C(O)NR⁴⁶(CR⁴⁶₂)_{q}P(O)(R⁴¹³)OR⁴¹⁰, —C(O)NR⁴⁶S(O)₂(CR⁴⁶₂)_{q}C(O)OR⁴¹⁰, —(CR⁴⁶₂)_{q}C(O)OR⁴¹⁰, —(CR⁴⁶₂)_{q}S(O)₂OR⁴¹⁰, —(CR⁴⁶₂)_{q}P(O)(R⁴¹³)(OR⁴¹⁰), —OC(O)NHOR⁴¹¹, —O(CR⁴⁶₂)_{q}C(O)OR⁴¹⁰, —OC(O)NHCN, —O(CR⁴⁶₂)_{q}S(O)₂OR⁴¹⁰, —O(CR⁴⁶₂)_{q}P(O)(R⁴¹³)(OR⁴¹⁰), —NR⁴⁶C(O)NHOR⁴¹¹, —NR⁴⁶C(O)NHCN, —C(O)NHS(O)₂R⁴¹², —OC(O)NHS(O)₂R⁴¹², —NR⁴⁶C(O)NHS(O)₂R⁴¹², —S(O)₂OR⁴¹⁰, —OS(O)₂OR⁴¹⁰, —NR⁴⁶S(O)₂OR⁴¹⁰, —NR⁴⁶S(O)OR⁴¹⁰, —NHS(O)₂R⁴¹⁴, —S(O)₂OR⁴¹⁰, —S(CR⁴⁶₂)_{q}C(O)OR⁴¹⁰, —S(CR⁴⁶₂)_{q}S(O)₂OR⁴¹⁰, —S(CR⁴⁶₂)_{q}P(O)(R⁴¹³)(OR⁴¹⁰), —OS(O)OR⁴¹⁰, —S(O)₂NHCN, —S(O)₂NHC(O)R⁴¹⁸, —S(O)₂NHS(O)₂R⁴¹², —OS(O)₂NHCN, —OS(O)₂NHS(O)₂R⁴¹², —OS(O)₂NHC(O)R⁴¹⁸, —NR⁴⁶S(O)₂NHCN, —NR⁴⁶S(O)₂NHC(O)R⁴¹⁸, —N(OH)C(O)R⁴¹⁵, —ONHC(O)R⁴¹⁵, —NR⁴⁶S(O)₂NHS(O)₂R⁴¹², —P(O)(R⁴¹³)(OR⁴¹⁰), —P(O)H(OR⁴¹⁰), —OP(O)(R⁴¹³)(OR⁴¹⁰), —NR⁴⁶P(O)(R⁴¹³)(OR⁴¹⁰) and tetrazole;

each R⁴⁶ is independently selected from hydrogen and C₁-C₆alkyl;

each R⁴⁹ is independently selected from the group consisting of halogen, cyano, —OH, —N(R⁴⁶)₂, C₁-C₄alkyl, C₁-C₄alkoxy, C₁-C₄haloalkyl and C₁-C₄haloalkoxy;

R⁴¹⁰ is selected from the group consisting of hydrogen, C₁-C₆alkyl, phenyl and benzyl, and wherein said phenyl or benzyl are optionally substituted by 1, 2 or 3 R⁴⁹ substituents, which may be the same or different;

R⁴¹¹ is selected from the group consisting of hydrogen, C₁-C₆alkyl, —C(O)OR⁴¹⁰, and phenyl, and wherein said phenyl is optionally substituted by 1, 2 or 3 R⁴⁹ substituents, which may be the same or different;

R⁴¹² is selected from the group consisting of C₁-C₆alkyl, C₃-C₆cycloalkyl, C₁-C₆haloalkyl, C₁-C₆alkoxy, —OH, —N(R⁴⁶)₂, phenyl, a 5- or 6-membered heteroaryl comprising 1, 2, 3 or 4 heteroatoms individually selected from N, O and S, and a 4- to 6-membered heterocyclyl comprising 1, 2 or 3 heteroatoms individually selected from N, O and S, and wherein said phenyl, heteroaryl or heterocyclyl moieties are optionally substituted by 1 or 2 R⁴²⁰ substituents;

R⁴¹³ is selected from the group consisting of —OH, C₁-C₆alkyl, C₁-C₆alkoxy and phenyl;

R⁴¹⁴ is selected from the group consisting of C₁-C₆alkyl, C₁-C₆haloalkyl, and N(R⁴⁶)₂;

R⁴¹⁵ is selected from the group consisting of C₁-C₆alkyl and phenyl, and wherein said phenyl is optionally substituted by 1, 2 or 3 R⁴⁹ substituents, which may be the same or different;

R⁴¹⁸ is selected from the group consisting of hydrogen, C₁-C₆alkyl, C₁-C₆haloalkyl, C₁-C₆alkoxy, —N(R⁴⁶)₂ and phenyl, and wherein said phenyl is optionally substituted by 1, 2 or 3 R⁴⁹ substituents, which may be the same or different;

each R⁴²⁰ is independently C₁-C₆ alkyl, C₁-C₆alkoxy, halogen, C₁-C₆haloalkyl, C₁-C₆haloalkoxy, or C₁-C₃alkoxyC₁-C₃alkyl;

R⁴²⁴ is a peptide moiety comprising 1, 2, or 3 amino acid moieties, each amino acid moiety independently selected from the group consisting of Ala, Cys, Asp, Glu, Phe, Gly, His, lie, Lys, Leu, Met, Asn, Pro, Gln, Arg, Ser, Thr, Val, Trp and Tyr, wherein said peptide moiety is bonded to the rest of the molecule via a nitrogen atom in the amino acid moiety;

R⁴²⁵ is phenyl optionally substituted by 1 or 2 R⁴⁹ substituents, or a 5- or 6-membered heteroaryl comprising 1, 2, 3 or 4 heteroatoms individually selected from N, O and S and optionally substituted by 1 or 2 R⁴⁹ substituents;

q is an integer of 1, 2 or 3;

each R⁵ is independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;

k is an integer of 0, 1, 2, 3, or 4;

each R⁶ is independently selected from hydrogen and C₁-C₆alkyl;

each R⁷ is independently selected from the group consisting of C₁-C₆alkyl, —S(O)₂R¹⁵, —C(O)R¹⁵, —C(O)OR¹⁵ and —C(O)NR¹⁶R¹⁷;

each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};

R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or

R^{7b} and R^{7c} together with the nitrogen atom to which they are attached form a 4- to 6-membered heterocyclyl ring which optionally comprises one additional heteroatom individually selected from N, O and S;

X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, and wherein the aforementioned CR¹R², Q and Z moieties may be attached at any position of said cycloalkyl, phenyl, heteroaryl or heterocyclyl moieties;

n is 0 or 1;

each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;

Z is selected from the group consisting of hydrogen, methoxy, —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;

R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;

R¹¹ is selected from the group consisting of hydrogen, C₁-C₆alkyl and phenyl, and wherein said phenyl is optionally substituted by 1, 2 or 3 R⁹ substituents, which may be the same or different;

R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;

R¹³ is selected from the group consisting of —OH, C₁-C₆alkyl, C₁-C₆alkoxy and phenyl;

R¹⁴ is C₁-C₆haloalkyl;

R¹⁵ is selected from the group consisting of C₁-C₆alkyl and phenyl, and wherein said phenyl is optionally substituted by 1, 2 or 3 R⁹ substituents, which may be the same or different;

R^{15a} is phenyl, wherein said phenyl is optionally substituted by 1, 2 or 3 R⁹ substituents, which may be the same or different;

R¹⁶ and R¹⁷ are independently selected from the group consisting of hydrogen and C₁-C₆alkyl; or R¹⁶ and R¹⁷ together with the nitrogen atom to which they are attached form a 4- to 6-membered heterocyclyl ring which optionally comprises one additional heteroatom individually selected from N, O and S;

R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different; and r is 0, 1 or 2; with the proviso that: (i) when A is —P(O)(OH)(OR⁴¹⁰) and R⁴¹⁰ is C₁-C₆alkyl, and R¹ and R² are both hydrogen, m is 0, and n is 0, then Z is not hydrogen, and (ii) the compound of formula (I) is not methyl 2,3-dimethylcinnolin-2-ium-4-carboxylate.

According to a second aspect of the invention, there is provided an agrochemical composition comprising a herbicidally effective amount of a compound of formula (I) and an agrochemically-acceptable diluent or carrier. Such an agricultural composition may further comprise at least one additional active ingredient.

According to a third aspect of the invention, there is provided a method of controlling or preventing undesirable plant growth, wherein a herbicidally effective amount of a compound of formula (I), or a composition comprising this compound as active ingredient, is applied to the plants, to parts thereof or the locus thereof.

According to a fourth aspect of the invention, there is provided the use of a compound of formula (I) as a herbicide.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, amino means an —NH₂ group.

As used herein, nitro means an —NO₂ group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl (Me), ethyl (Et), n-propyl, 1-methylethyl (iso-propyl), n-butyl, and 1-dimethylethyl (t-butyl).

As used herein, the term “C₁-C₆alkoxy” refers to a radical of the formula —ORₐ where Rₐ is a C₁-C₆alkyl radical as generally defined above. C₁-C₄alkoxy is to be construed accordingly. Examples of C₁₋₄alkoxy include, but are not limited to, methoxy, ethoxy, propoxy, iso-propoxy and t-butoxy.

As used herein, the term “C₁-C₆haloalkyl” refers to a C₁-C₆alkyl radical as generally defined above substituted by one or more of the same or different halogen atoms. C₁-C₄haloalkyl is to be construed accordingly. Examples of C₁-C₆haloalkyl include, but are not limited to chloromethyl, fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. C₂-C₄alkenyl is to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical as generally defined above substituted by one or more of the same or different halogen atoms. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₂-C₄alkynyl is to be construed accordingly. Examples of C₂-C₆alkynyl include, but are not limited to, prop-1-ynyl, propargyl (prop-2-ynyl) and but-1-ynyl.

As used herein, the term “C₁-C₆haloalkoxy” refers to a C₁-C₆alkoxy group as defined above substituted by one or more of the same or different halogen atoms. C₁-C₄haloalkoxy is to be construed accordingly. Examples of C₁-C₆haloalkoxy include, but are not limited to, fluoromethoxy, difluoromethoxy, fluoroethoxy, trifluoromethoxy and trifluoroethoxy.

As used herein, the term “C₁-C₃haloalkoxyC₁-C₃alkyl” refers to a radical of the formula R_{b}—O—Rₐ— where R_{b} is a C₁-C₃haloalkyl radical as generally defined above, and Rₐ is a C₁-C₃alkylene radical as generally defined above.

As used herein, the term “C₁-C₃alkoxyC₁-C₃alkyl” refers to a radical of the formula R_{b}—O—Rₐ— where R_{b} is a C₁-C₃alkyl radical as generally defined above, and Rₐ is a C₁-C₃alkylene radical as generally defined above.

As used herein, the term “C₃-C₆alkenyloxy” refers to a radical of the formula —ORₐ where Rₐ is a C₃-C₆alkenyl radical as generally defined above.

As used herein, the term “C₃-C₆alkynyloxy” refers to a radical of the formula —ORₐ where Rₐ is a C₃-C₆alkynyl radical as generally defined above.

As used herein, the term “hydroxyC₁-C₆alkyl” refers to a C₁-C₆alkyl radical as generally defined above substituted by one or more hydroxy groups.

As used herein, the term “C₁-C₆alkylcarbonyl” refers to a radical of the formula —C(O)Rₐ where Rₐ is a C₁-C₆alkyl radical as generally defined above.

As used herein, the term “C₁-C₆alkoxycarbonyl” refers to a radical of the formula —C(O)ORₐ where Rₐ is a C₁-C₆alkyl radical as generally defined above.

As used herein, the term “aminocarbonyl” refers to a radical of the formula —C(O)NH₂.

As used herein, the term “C₁-C₆alkylaminocarbonyl” refers to a radical of the formula —C(O)NHRₐ where Rₐ is a C₁-C₆alkyl radical as generally defined above.

As used herein, the term “di-C₁-C₆alkylaminocarbonyl” refers to a radical of the formula —C(O)NRₐ(Rₐ) where each Rₐ is independently a C₁-C₆alkyl radical as generally defined above.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to, cyclopropyl, cyclobutyl, cyclopentyl and cyclohexyl.

As used herein, the term “C₃-C₆halocycloalkyl” refers to a C₃-C₆cycloalkyl radical as generally defined above substituted by one or more of the same or different halogen atoms. C₃-C₄halocycloalkyl is to be construed accordingly.

As used herein, the term “C₃-C₆cycloalkoxy” refers to a radical of the formula —ORₐ where Rₐ is a C₃-C₆cycloalkyl radical as generally defined above.

As used herein, except where explicitly stated otherwise, the term “heteroaryl” refers to a 5- or 6-membered monocyclic aromatic ring which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heterocyclyl” or “heterocyclic” refers to a stable 3- to 6-membered non-aromatic monocyclic ring radical which comprises 1, 2, or 3 heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or δ-lactamyl.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

For example a compound of formula (I) wherein A or Z comprises an acidic proton, may exist as a zwitterion, e.g as a compound of formula (I-I) or formula (I-III), or as an agronomically acceptable salt, e.g. as a compound of formula (I-II) as shown below:

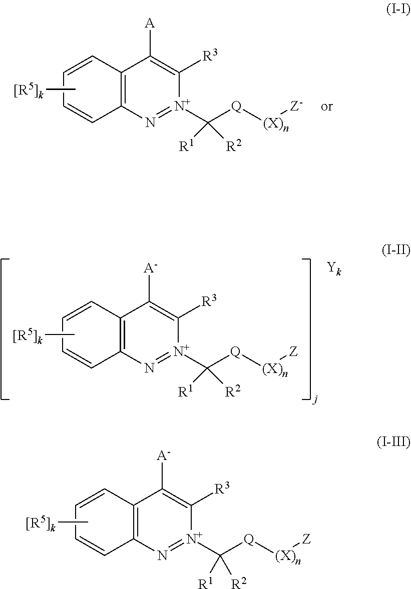

wherein, Y represents an agronomically acceptable anion and j and k represent integers that may be selected from 1, 2 or 3, dependent upon the charge of the respective anion Y.

A compound of formula (I) may also exist as an agronomically acceptable salt of a zwitterion in the form of a compound of formula (I-IV) as shown below:

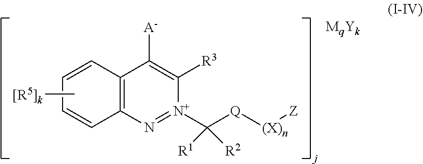

wherein, Y represents an agronomically acceptable anion, M represents an agronomically acceptable cation (in addition to the cinnolinium cation) and the integers j, k and q may be selected from 1, 2 or 3, dependent upon the charge of the respective anion Y and respective cation M.

Thus where a compound of formula (I) is drawn in protonated form herein, the skilled person would appreciate that it could equally be represented in unprotonated or salt form with one or more relevant counter ions.

In one embodiment of the invention there is provided a compound of formula (I-II) or formula (I-IV) wherein k is 2, j is 1 and Y is selected from the group consisting of halogen, trifluoroacetate and pentafluoropropionate. In this embodiment a nitrogen atom comprised in R¹, R², R³, R⁴, R⁵, A, Q, Z or X may be protonated.

Suitable agronomically acceptable salts of the present invention, represented by an anion Y, include but are not limited chloride, bromide, iodide, fluoride, 2-naphthalenesulfonate, acetate, adipate, methoxide, ethoxide, propoxide, butoxide, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydrogen sulfate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, pentafluoropropionate, perchlorate, phosphate, propionate, propylsulfate, propylsulfonate, succinate, sulfate, tartrate, tosylate, tridecylate, triflate, trifluoroacetate, undecylinate and valerate.

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

Preferred compounds of formula (I), wherein A and/or Z comprise(s) an acidic proton, can be represented as either (1-1), (I-II), (I-III) or (I-IV). For compounds of formula (I-II) or (I-IV) emphasis is given to salts when Y is chloride, bromide, iodide, hydroxide, bicarbonate, acetate, pentafluoropropionate, perchlorate, triflate, trifluoroacetate, methylsulfate, tosylate and nitrate, wherein j and k are 1. Preferably, Y is chloride, bromide, iodide, hydroxide, bicarbonate, acetate, trifluoroacetate, methylsulfate, tosylate and nitrate, wherein j and k are 1. For compounds of formula (I-II) or (I-IV) emphasis is also given to salts when Y is carbonate and sulfate, wherein j is 2 and k is 1, and when Y is phosphate, wherein j is 3 and k is 1.

Where appropriate compounds of formula (I) may also be in the form of (and/or be used as) an N-oxide.

Compounds of formula (I) wherein m is 0 and n is 0 may be represented by a compound of formula (I-Ia) as shown below:

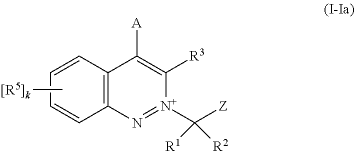

(I-Ia) wherein k, R¹, R², R³, A, R⁵ and Z are as defined for compounds of formula (I).

Compounds of formula (I) wherein m is 1 and n is 0 may be represented by a compound of formula (I-Ib) as shown below:

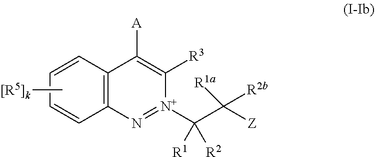

(I-Ib) wherein k, R¹, R², R^{1a}, R^{2b}, R³, A, R⁵ and Z are as defined for compounds of formula (I).

Compounds of formula (I) wherein m is 2 and n is 0 may be represented by a compound of formula (I-Ic) as shown below:

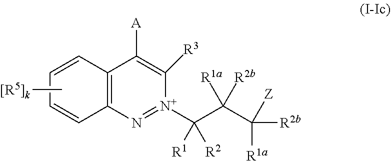

wherein k, R¹, R², R^{1a}, R^{2b}, R³, A, R⁵ and Z are as defined for compounds of formula (I).

Compounds of formula (I) wherein m is 3 and n is 0 may be represented by a compound of formula (I-Id) as shown below:

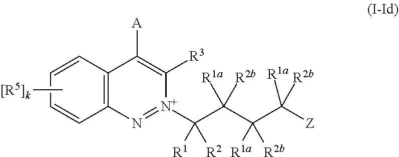

(I-Id) wherein k, R¹, R², R^{1a}, R^{2b}, R³, A, R⁵ and Z are as defined for compounds of formula (I).

The following list provides definitions, including preferred definitions, for the substituents R¹, R², R^{1a}, R^{2b}, R³, R⁵, R⁶, R⁷, R^{7a}, R^{7b}, R^{7c}, R⁹, R¹⁰, R¹¹, R¹², R¹³, R¹⁴, R¹¹, R^{15a}, R¹⁶, R¹⁷, R¹⁸, R⁴⁶, R⁴⁹, R⁴¹⁰, R⁴¹¹, R⁴¹², R⁴¹³, R⁴¹⁴, R⁴¹⁵, R⁴¹⁸, R⁴²⁰, R⁴²⁴, R⁴²⁵, A, Q, X, and Z, and integers k, m, n, q and r, as used herein. For any one of these substituents and/or integers, any of the definitions given below may be combined with that of any other substituent and/or integer given below or elsewhere in this document.

As defined supra, A is selected from the group consisting of —C(O)OR⁴¹⁰, —CHO, —C(O)R⁴²⁴, —C(O)NHOR⁴¹¹, —C(O)NHCN, —C(O)NHR⁴²⁵, —S(O)₂NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶(CR⁴⁶₂)_{q}—S(O)₂OR⁴¹⁰, —C(O)NR⁴⁶(CR⁴⁶₂)_{q}P(O)(R⁴¹³)OR⁴¹⁰, —C(O)NR⁴⁶S(O)₂(CR⁴⁶₂)_{q}C(O)OR⁴¹⁰, —(CR⁴⁶₂)_{q}C(O)OR⁴¹⁰, —(CR⁴⁶₂)_{q}S(O)₂OR⁴¹⁰, —(CR⁴⁶₂)_{q}P(O)(R⁴¹³)(OR⁴¹⁰), —OC(O)NHOR⁴¹¹, —O(CR⁴⁶₂)_{q}C(O)OR⁴¹⁰, —OC(O)NHCN, —O(CR⁴⁶₂)_{q}S(O)₂OR⁴¹⁰, O(CR⁴⁶₂)_{q}P(O)(R⁴¹³)(OR⁴¹⁰), —NR⁴⁶C(O)NHOR⁴¹¹, —NR⁴⁶C(O)NHCN, —C(O)NHS(O)₂R⁴¹², —OC(O)NHS(O)₂R⁴¹², —NR⁴⁶C(O)NHS(O)₂R⁴¹², —S(O)₂OR⁴¹⁰, —OS(O)₂OR⁴¹⁰, —NR⁴⁶S(O)₂OR⁴¹⁰, —NR⁴⁶S(O)OR⁴¹⁰, —NHS(O)₂R⁴¹⁴, —S(O)₂OR⁴¹⁰, —S(CR⁴⁶₂)_{q}C(O)OR⁴¹⁰, —S(CR⁴⁶₂)_{q}S(O)₂OR⁴¹⁰. —S(CR⁴⁶₂)_{q}P(O)(R⁴¹³)(OR⁴¹⁰), —OS(O)OR⁴¹⁰, —S(O)₂NHCN, —S(O)₂NHC(O)R⁴¹⁸, —S(O)₂NHS(O)₂R⁴¹², —OS(O)₂NHCN, —OS(O)₂NHS(O)₂R⁴¹², —OS(O)₂NHC(O)R⁴¹⁸, —NR⁴⁶S(O)₂NHCN, —NR⁴⁶S(O)₂NHC(O)R⁴¹⁸, —N(OH)C(O)R⁴¹⁵, —ONHC(O)R⁴¹⁵, —NR⁴⁶S(O)₂NHS(O)₂R⁴¹², —P(O)(R⁴¹³)(OR⁴¹⁰), —P(O)H(OR⁴¹⁰), —OP(O)(R⁴¹³)(OR⁴¹⁰), —NR⁴⁶P(O)(R⁴¹³)(OR⁴¹⁰) and tetrazole, wherein each R⁴⁶ is independently selected from hydrogen and C₁-C₆alkyl; each R⁴⁹ is independently selected from the group consisting of halogen, cyano, —OH, —N(R⁴⁶)₂, C₁-C₄alkyl, C₁-C₄alkoxy, C₁-C₄haloalkyl and C₁-C₄haloalkoxy; R⁴¹⁰ is selected from the group consisting of hydrogen, C₁-C₆alkyl, phenyl and benzyl, and wherein said phenyl or benzyl are optionally substituted by 1, 2 or 3 R⁴⁹ substituents, which may be the same or different; R⁴¹¹ is selected from the group consisting of hydrogen, C₁-C₆alkyl, —C(O)OR⁴¹⁰, and phenyl, and wherein said phenyl is optionally substituted by 1, 2 or 3 R⁴⁹ substituents, which may be the same or different; R⁴¹² is selected from the group consisting of C₁-C₆alkyl, C₃-C₆cycloalkyl, C₁-C₆haloalkyl, C₁-C₆alkoxy, —OH, —N(R⁴⁶)₂, phenyl, a 5- or 6-membered heteroaryl comprising 1, 2, 3 or 4 heteroatoms individually selected from N, O and S, and a 4- to 6-membered heterocyclyl comprising 1, 2 or 3 heteroatoms individually selected from N, O and S, and wherein said phenyl, heteroaryl or heterocyclyl moieties are optionally substituted by 1 or 2 R⁴²⁰ substituents; R⁴¹³ is selected from the group consisting of —OH, C₁-C₆alkyl, C₁-C₆alkoxy and phenyl; R⁴¹⁴ is selected from the group consisting of C₁-C₆alkyl, C₁-C₆haloalkyl, and N(R⁴⁶)₂; R⁴¹⁵ is selected from the group consisting of C₁-C₆alkyl and phenyl, and wherein said phenyl is optionally substituted by 1, 2 or 3 R⁴⁹ substituents, which may be the same or different; R⁴¹⁸ is selected from the group consisting of hydrogen, C₁-C₆alkyl, C₁-C₆haloalkyl, C₁-C₆alkoxy, —N(R⁴⁶)₂ and phenyl, and wherein said phenyl is optionally substituted by 1, 2 or 3 R⁴⁹ substituents, which may be the same or different; each R⁴²⁰ is independently C₁-C₆ alkyl, C₁-C₆alkoxy, halogen, C₁-C₆haloalkyl, C₁-C₆haloalkoxy, or C₁-C₃alkoxyC₁-C₃alkyl; R⁴²⁴ is a peptide moiety comprising 1, 2, or 3 amino acid moieties, each amino acid moiety independently selected from the group consisting of Ala, Cys, Asp, Glu, Phe, Gly, His, lie, Lys, Leu, Met, Asn, Pro, Gln, Arg, Ser, Thr, Val, Trp and Tyr, wherein said peptide moiety is bonded to the rest of the molecule via a nitrogen atom in the amino acid moiety; R⁴²⁵ is phenyl optionally substituted by 1 or 2 R⁴⁹ substituents, or a 5- or 6-membered heteroaryl comprising 1, 2, 3 or 4 heteroatoms individually selected from N, O and S and optionally substituted by 1 or 2 R⁴⁹ substituents.

Preferably A is selected from the group consisting of: —C(O)OR⁴¹⁰, —C(O)NHOR⁴¹¹, —C(O)NHR⁴²⁵, —S(O)₂NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶S(O)₂(CR⁴⁶₂)_{q}C(O)OR⁴¹⁰, —(CR⁴⁶₂)_{q}C(O)OR⁴¹⁰, —(CR⁴⁶₂)_{q}P(O)(R⁴¹³)(OR⁴¹⁰), —OC(O)NHOR⁴¹¹, —O(CR⁴⁶₂)_{q}C(O)OR⁴¹⁰, —OC(O)NHCN, —O(CR⁴⁶₂)_{q}S(O)₂OR⁴¹⁰, —O(CR⁴⁶₂)_{q}P(O)(R⁴¹³)(OR⁴¹⁰), —S(O)₂OR⁴¹⁰, —S(CR⁴⁶₂)_{q}C(O)OR⁴¹⁰, —S(CR⁴⁶₂)_{q}S(O)₂OR⁴¹⁰, —P(O)(R⁴¹³)(OR⁴¹⁰), —P(O)H(OR⁴¹⁰), —OP(O)(R⁴¹³)(OR⁴¹⁰), and —NR⁴⁶P(O)(R⁴¹³)(OR⁴¹⁰).

More preferably A is selected from the group consisting of: —C(O)OR⁴¹⁰, —C(O)NHOR⁴¹¹, —C(O)NHR⁴²⁵, —C(O)NHS(O)₂R⁴¹⁴, —C(O)NR⁴⁶(CR⁴⁶₂)_{q}C(O)OR⁴¹⁰, C(O)NR⁴⁶S(O)₂(CR⁴⁶₂)_{q}C(O)—OR⁴¹⁰, —(CR⁴⁶₂)_{q}C(O)OR⁴¹⁰, —(CR⁴⁶₂)_{q}P(O)(R⁴¹³)(OR⁴¹⁰), —S(O)₂OR⁴¹⁰, —S(CR⁴⁶₂)_{q}C(O)OR⁴¹⁰, —O(CR⁴⁶₂)_{q}C(O)OR⁴¹⁰, and —P(O)(R⁴¹³)(OR⁴¹⁰).

Preferably each R⁴⁶ is independently selected from the group consisting of hydrogen and C₁-C₃ alkyl, and more preferably from the group consisting of hydrogen, methyl, and ethyl. Most preferably each R⁴⁶ is independently selected from hydrogen and methyl.

Preferably each R⁴⁹ is independently selected from the group consisting of C₁-C₄alkyl, C₁-C₄haloalkyl, C₁-C₄alkoxy, C₁-C₄haloalkoxy, and halogen. More preferably, each R⁴⁹ is independently selected from the group consisting of C₁-C₂alkyl, C₁-C₂haloalkyl, C₁-C₂alkoxy, C₁-C₂haloalkoxy, and halogen.

More preferably still, each R⁴⁹ is independently selected from the group consisting of methyl, ethyl and halogen. Most preferably each R⁴⁹ is independently selected from the group consisting of methyl, chloro and fluoro. In preferred embodiments, where R⁴⁹ is present, there will be either 1 or 2 R⁴⁹ substituents. In other preferred embodiments, R⁴⁹ is absent and the relevant cyclic group is unsubstituted.

The integer q is preferably 1 or 2.

Preferably R⁴¹⁰ is selected from the group consisting of hydrogen and C₁-C₆ alkyl. In particular, R⁴¹⁰ may be hydrogen, methyl, ethyl, n-propyl, cylco-propyl, iso-propyl, or n-butyl, sec-butyl, tert-butyl or iso-butyl.

R⁴¹¹ is preferably hydrogen or C₁-C₆alkyl, more preferably hydrogen or C₁-C₃alkyl, and most preferably hydrogen or methyl.

R⁴¹³ is preferably —OH, C₁-C₆alkyl, or C₁-C₆alkoxy. More preferably R⁴¹³ is —OH, C₁-C₄alkyl, or C₁-C₄alkoxy. Most preferably R⁴¹³ is selected from the group consisting of —OH, methoxy, ethoxy, isopropyloxy, methyl, ethyl, n-propyl, i-propyl, and i-butyl.

R⁴¹⁴ is preferably selected from the group consisting of C₁-C₄alkyl, C₁-C₄haloalkyl, and N(R⁴⁶)₂, wherein each R⁴⁶ may be the same or different. More preferably R⁴¹⁴ is selected from the group consisting of C₁-C₄alkyl, C₁-C₃haloalkyl, and N(R⁴⁶)₂.

R⁴²⁵ is preferably selected from the group consisting of phenyl, thiophene, thiazole, imidazole, pyrazole, isothiazole, triazole, tetrazole, pyridazine, pyrimidine, pyrazine, and triazine, each optionally substituted by 1 or 2 R⁴⁹ substituents. More preferably R⁴²⁵ is selected from the group consisting of phenyl, thiophene, thiazole, triazole, and tetrazole, each optionally substituted by 1 R⁴⁹ substituent.

R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵. Preferably, R¹ is selected from the group consisting of hydrogen, halogen, C₁-C₆alkyl, C₁-C₆fluoroalkyl, —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵. More preferably, R¹ is selected from the group consisting of hydrogen, halogen, C₁-C₆alkyl, C₁-C₆fluoroalkyl, —OR⁷ and —N(R^{7a})₂. Even more preferably, R¹ is selected from the group consisting of hydrogen, C₁-C₆alkyl, —OR⁷ and —N(R^{7a})₂. Even more preferably still, R¹ is hydrogen or C₁-C₆alkyl. Yet even more preferably still, R¹ is hydrogen or C₁-C₃alkyl (preferably methyl). Most preferably R¹ is hydrogen.

R² is selected from the group consisting of hydrogen, halogen, C₁-C₆alkyl and C₁-C₆haloalkyl. Preferably, R² is selected from the group consisting of hydrogen, halogen, C₁-C₆alkyl and C₁-C₆fluoroalkyl. More preferably, R² is hydrogen or C₁-C₆alkyl. Even more preferably, R² is hydrogen or C₁-C₃alkyl (preferably methyl). Most preferably R² is hydrogen.

Wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl. Preferably, when R¹ is selected from the group consisting of —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵, R² is selected from the group consisting of hydrogen and methyl.

Alternatively, R¹ and R² together with the carbon atom to which they are attached form a C₃-C₆cycloalkyl ring or a 3- to 6-membered heterocyclyl, which comprises 1 or 2 heteroatoms individually selected from N and O. Preferably, R¹ and R² together with the carbon atom to which they are attached form a C₃-C₆cycloalkyl ring. More preferably, R¹ and R² together with the carbon atom to which they are attached form a cyclopropyl ring.

In one embodiment R¹ and R² are independently selected from the group consisting of hydrogen and C₁-C₃alkyl.

In another embodiment R¹ and R² are hydrogen.

In another embodiment R¹ is methyl and R² is hydrogen.

In another embodiment R¹ is methyl and R² is methyl.

Q is (CR^{1a}R^{2b})ₘ.

As stated herein, m is an integer of 0, 1, 2 or 3. In a set of embodiments, m is 0. In another set of embodiments, m is 1. In still a further set of embodiments, m is 3.

Each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵. Preferably, each Ria and R^{2b} are independently selected from the group consisting of hydrogen, halogen, C₁-C₆alkyl, C₁-C₆fluoroalkyl, —OH, —NH₂ and —NHR⁷. More preferably, each R^{1a} and R^{2b} are independently selected from the group consisting of hydrogen, halogen, C₁-C₆alkyl, —OH and —NH₂. Even more preferably, each Ria and R^{2b} are independently selected from the group consisting of hydrogen, halogen and C₁-C₄ alkyl.

In one set of embodiments when m is 1, R^{1a} is preferably selected from hydrogen and halogen, and R^{2b} is preferably independently selected from hydrogen, halogen, and C₁-C₄alkyl.

In a further set of embodiments, when m is 3, Q is —CH₂—CH₂—CH(Z)(n-butyl) i.e. each R^{1a} is hydrogen, and each R^{2b} is independently selected from hydrogen or C₄alkyl.

Alternatively, each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O. Preferably, each R^{1a} and R^{2b} together with the carbon atom to which they are attached form a C₃-C₆cycloalkyl ring. More preferably, each R^{1a} and R^{2b} together with the carbon atom to which they are attached form a cyclopropyl ring.

R³ is selected from the group consisting of hydrogen, halogen, cyano, nitro, —S(O)ᵣR¹⁵, C₁-C₆alkyl, C₁-C₆halooalkyl, C₁-C₆haloalkoxy, C₁-C₆alkoxy, C₃-C₆cycloalkyl, —N(R⁶)₂, phenyl, a 5-or 6-membered heteroaryl comprising 1, 2, 3 or 4 heteroatoms individually selected from N, O and S, and a 4- to 6-membered heterocyclyl comprising 1, 2 or 3 heteroatoms individually selected from N, O and S, and wherein said phenyl, heteroaryl or heterocyclyl moieties are optionally substituted by 1 or 2 R⁹ substituents; Preferably, R³ is selected from the group consisting of hydrogen, halogen and C₁-C₆alkyl, phenyl and thiazole, wherein said phenyl or thiazole is optionally substituted by 1 or 2 R⁹, which may be the same or different. More preferably, R³ is selected from the group consisting of hydrogen, C₁-C₃alkyl, thiazole and phenyl. Even more preferably, R³ is selected from the group consisting of hydrogen, methyl, thiazole and phenyl.

As defined herein, k is 0, 1, 2, 3 or 4. Preferably k is 0, 1 or 2. More preferably k is 0 or 1. In one embodiment k is 0. In another embodiment k is 1.

When k is 1 or 2, each R⁵ is independently selected from the group consisting of halogen, nitro, cyano, —NH₂, —NR⁶R⁷, —OH, —OR⁷, —S(O)ᵣR¹², —NR⁶S(O)ᵣR¹², C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₆haloalkoxy, C₁-C₃haloalkoxyC₁-C₃alkyl-, C₁-C₆alkoxycarbonyl, C₃-C₆alkenyloxy, C₃-C₆alkynyloxy, C₁-C₆alkylcarbonyl, C₁-C₆alkylaminocarbonyl, di-C₁-C₆alkylaminocarbonyl, —C(R⁸)═NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different.

Preferably when k is 1 or 2, each R⁵ is independently selected from the group consisting of halogen, nitro, cyano, —NH₂, —NR⁶R⁷, —OH, —OR⁷, C₁-C₆alkyl, C₁-C₆haloalkyl, C₃-C₆cycloalkyl, C₃-C₆cycloalkoxy, C₂-C₆alkenyl, C₂-C₆haloalkenyl, C₂-C₆alkynyl, C₁-C₆haloalkoxy, C₁-C₃haloalkoxyC₁-C₃alkyl-, C₁-C₆alkoxycarbonyl, C₁-C₆alkylcarbonyl, C₁-C₆alkylaminocarbonyl, di-C₁-C₆alkylaminocarbonyl, —C(R⁸)=NOR⁸, phenyl and heteroaryl, wherein the heteroaryl moiety is a 5- or 6-membered monocyclic aromatic ring which comprises 1, 2, 3 or 4 heteroatoms individually selected from N, O and S, and wherein any of said phenyl or heteroaryl moieties are optionally substituted by 1, 2 or 3 R⁹ substituents, which may be the same or different.

More preferably, when k is 1 or 2, each R⁵ is independently selected from the group consisting of halogen, cyano, —NH₂, —NR⁶R⁷, —OH, —OR⁷, C₁-C₃alkyl, C₁-C₃haloalkyl, C₃-C₆cycloalkyl, C₁-C₃haloalkoxy, C₂-C₄alkenyl, C₂-C₄alkynyl, C₁-C₃alkoxycarbonyl, C₁-C₃alkylaminocarbonyl, di-C₁-C₃alkylaminocarbonyl and phenyl, wherein said phenyl is optionally substituted by 1, 2 or 3 R⁹ substituents, which may be the same or different.

Further more preferably, when k is 1 or 2, each R⁵ is independently selected from the group consisting of halogen, cyano, —NR⁶R⁷, —OR⁷, C₁-C₃alkyl, C₁-C₃haloalkyl, C₁-C₃alkoxycarbonyl, C₁-C₃alkylaminocarbonyl, di-C₁-C₃alkylaminocarbonyl and phenyl.

Further more preferably still, when k is 1 or 2, each R⁵ is independently selected from the group consisting of chloro, fluoro, bromo, iodo, cyano, —NHC(O)Me, methoxy, methyl, trifluoromethyl, methoxycarbonyl, di-methylaminocarbonyl and phenyl.

Yet further more preferably still, when k is 1 or 2, each R⁵ is independently selected from the group consisting of chloro, fluoro, bromo, iodo, —NHC(O)Me, methoxy, methyl and di-methylaminocarbonyl.

Alternatively, when k is 3 or 4, each R⁵ is independently selected from the group consisting of halogen, C₁-C₆alkyl, C₁-C₆haloalkyl, C₁-C₆alkoxy and C₁-C₆haloalkoxy. Preferably each R⁵ is independently selected from the group consisting of chloro, fluoro, bromo, iodo, methoxy, methyl and trifluoromethyl. More preferably each R⁵ is independently selected from the group consisting of chloro, fluoro, methoxy and methyl. Even more preferably each R⁵ is independently selected from the group consisting of chloro, fluoro and methyl. Most preferably each R⁵ is methyl.

Each R⁶ is independently selected from hydrogen and C₁-C₆alkyl. Preferably, each R⁶ is independently selected from hydrogen and methyl.

R⁷ is independently selected from the group consisting of C₁-C₆alkyl, —S(O)₂R¹⁵, —C(O)R¹⁵, —C(O)OR¹⁵ and —C(O)NR¹⁶R¹⁷. Preferably, each R⁷ is independently selected from the group consisting of C₁-C₆alkyl, —C(O)R¹⁵ and —C(O)NR¹⁶R¹⁷. More preferably, each R⁷ is C₁-C₆alkyl. Most preferably, each R⁷ is methyl.

Each R^{7a} is independently selected from the group consisting of —S(O)₂R¹⁵, —C(O)R¹⁵, —C(O)OR⁵—C(O)NR¹⁶R¹⁷ and —C(O)NR⁶R^{15a}. Preferably, each R^{7a} is independently —C(O)R¹⁵ or —C(O)NR¹⁶R¹⁷.

R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different. Preferably, R^{7b} and R^{7c} are independently selected from the group consisting of C₁-C₆alkyl, —C(O)R¹⁵ and —C(O)NR¹⁶R¹⁷. More preferably, R^{7b} and R^{7c} are C₁-C₆alkyl. Most preferably, R^{7b} and R^{7c} are methyl.

Alternatively, R^{7b} and R^{7c} together with the nitrogen atom to which they are attached form a 4- to 6-membered heterocyclyl ring which optionally comprises one additional heteroatom individually selected from N, O and S. In such embodiments, preferably, R^{7b} and R^{7c} together with the nitrogen atom to which they are attached form a 5- to 6-membered heterocyclyl ring which optionally comprises one additional heteroatom individually selected from N and O. More preferably in such embodiments, R^{7b} and R^{7c} together with the nitrogen atom to which they are attached form an pyrrolidyl, oxazolidinyl, imidazolidinyl, piperidyl, piperazinyl or morpholinyl group.

Each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy. Preferably, each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy. More preferably, each R⁹ is independently selected from the group consisting of halogen, C₁-C₄alkyl, C₁-C₄alkoxy and C₁-C₄haloalkyl. Even more preferably, each R⁹ is independently selected from the group consisting of halogen and C₁-C₄alkyl.

Each R⁸ is independently selected from the group consisting of hydrogen and C₁-C₄alkyl. Preferably, each R⁸ is independently selected from the group consisting of hydrogen and methyl. More preferably, each R⁸ is methyl.

X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, which may be the same or different, and wherein the aforementioned CR¹R², Q and Z moieties may be attached at any position of said cycloalkyl, phenyl, heteroaryl or heterocyclyl moieties.

Preferably, X is selected from the group consisting of phenyl and a 4- to 6-membered heterocyclyl, which comprises 1 or 2 heteroatoms individually selected from N and O, and wherein said phenyl or heterocyclyl moieties are optionally substituted by 1 or 2 R⁹ substituents, which may be the same or different, and wherein the aforementioned CR¹R², Q and Z moieties may be attached at any position of said phenyl or heterocyclyl moieties.

More preferably, X is phenyl or a 5-membered heterocyclyl, which comprises 1 or 2 heteroatoms individually selected from N and O, and wherein said phenyl and heterocyclyl moieties are optionally substituted by 1 or 2 R⁹ substituents, which may be the same or different, and wherein the aforementioned CR¹R², Q and Z moieties may be attached at any position of said phenyl or heterocyclyl moieties.

In one embodiment, X is a 5-membered heterocyclyl, which comprises 1 heteroatom, wherein said heteroatom is N, and wherein the aforementioned CR¹R², Q and Z moieties may be attached at any position of said heterocyclyl moiety. Preferably, X is a 5-membered heterocyclyl, which comprises 1 heteroatom, wherein said heteroatom is N, and wherein the aforementioned CR¹R² and Q moieties are attached adjacent to the N atom and the Z moiety is attached to the N atom.

In another embodiment, X is phenyl optionally substituted by 1 or 2 R⁹ substituents, which may be the same or different, and wherein the aforementioned CR¹R², Q and Z moieties may be attached at any position of said phenyl moiety. Preferably, X is phenyl and the aforementioned CR¹R² and Q moieties are attached in a position para to the Z moiety.

As stated herein, n is 0 or 1. Preferably, n is 0.

Group Z is defined herein as being selected from the group consisting of hydrogen, methoxy, —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂₀R¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole,

In one set of embodiments, Z is Z1 and in a second set of embodiments, Z is Z2.

Z1 is selected from the group consisting of hydrogen, —CH₂OH, and methoxy.

Z2 is selected from the group consisting of —C(O)OR¹⁰, —CHO, —C(O)NHOR¹¹, —C(O)NHCN, —OC(O)NHOR¹¹, —OC(O)NHCN, —NR⁶C(O)NHOR¹¹, —NR⁶C—(O)NHCN, —C(O)NHS(O)₂R¹², —OC(O)NHS(O)₂R¹², —NR⁶C(O)NHS(O)₂R¹², —S(O)₂OR¹⁰, —OS—(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;

More preferably, Z2 is selected from the group consisting of —C(O)OR¹⁰, —CH₂OH, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰, —P(O)(R¹³)(OR¹⁰) and tetrazole.

Even more preferably Z2 is selected from the group consisting of —C(O)OH, —C(O)OCH₃, —C(O)OCH(CH₃)₂, —C(O)OC(CH₃)₃, —CH₂OH, —C(O)NHOCH₃, —C(O)NHS(O)₂CH₃, —C(O)NHS(O)₂N(CH₃)₂, —S(O)₂OH, —OS(O)₂OH, —NHS(O)₂OH, —NHS(O)₂CF₃, —P(O)(OH)(OH), —P(O)(OH)(OCH₃), —P(O)(OCH₃)(OCH₃), —P(O)(OH)(OCH₂CH₃), —P(O)(OCH₂CH₃)(OCH₂CH₃) and tetrazole.

Even more preferably still Z2 is selected from the group consisting of —C(O)OH, —C(O)OCH3, —C(O)NHS(O)₂CH₃, —S(O)₂OH, and —OS(O)₂OH.

Most preferably Z2 is —C(O)OH or —S(O)₂OH.

R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different. Preferably, R¹⁰ is selected from the group consisting of hydrogen, C₁-C₄alkyl, phenyl and benzyl. More preferably, R¹⁰ is selected from the group consisting of hydrogen and C₁-C₃alkyl. Most preferably, R¹⁰ is hydrogen or methyl.

R¹¹ is selected from the group consisting of hydrogen, C₁-C₆alkyl and phenyl, and wherein said phenyl is optionally substituted by 1, 2 or 3 R⁹ substituents, which may be the same or different. Preferably, R¹¹ is selected from the group consisting of hydrogen, C₁-C₆alkyl and phenyl. More preferably, R¹¹ is selected from the group consisting of hydrogen and C₁-C₆alkyl. Even more preferably, R¹¹ is C₁-C₆alkyl. Most preferably, R¹¹ is methyl.

R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different. Preferably, R¹² is selected from the group consisting of C₁-C₆alkyl, C₁-C₆haloalkyl, C₁-C₆alkoxy, —OH, —N(R⁶)₂ and phenyl. More preferably, R¹² is selected from the group consisting of C₁-C₆alkyl, C₁-C₆haloalkyl and —N(R⁶)₂. Even more preferably, R¹² is selected from the group consisting of C₁-C₃alkyl, —N(Me)₂ and trifluoromethyl. More preferably still R¹² is C₁-C₃alkyl, and most preferably R¹² is methyl.

R¹³ is selected from the group consisting of —OH, C₁-C₆alkyl, C₁-C₆alkoxy and phenyl. Preferably R¹³ is selected from the group consisting of —OH, C₁-C₆alkyl and C₁-C₆alkoxy. More preferably, R¹³ is selected from the group consisting of —OH and C₁-C₆alkoxy. Even more preferably, R¹³ is selected from the group consisting of —OH, methoxy and ethoxy. Most preferably, R¹³ is —OH.

R¹⁴ is C₁-C₆haloalkyl. Preferably, R¹⁴ is trifluoromethyl.

R¹⁵ is selected from the group consisting of C₁-C₆alkyl, phenyl and benzyl, and wherein said phenyl or benzyl are optionally substituted by 1, 2 or 3 R⁹ substituents, which may be the same or different. Preferably, R¹⁵ is selected from the group consisting of C₁-C₆alkyl, phenyl and benzyl. More preferably, R¹⁵ is C₁-C₆alkyl. Most preferably R¹⁵ is methyl.

R^{15a} is phenyl, wherein said phenyl is optionally substituted by 1, 2 or 3 R⁹ substituents, which may be the same or different. Preferably, R^{15a} is phenyl optionally substituted by 1 R⁹ substituent. More preferably, R^{15a} is phenyl.

R¹⁶ and R¹⁷ are independently selected from the group consisting of hydrogen and C₁-C₆alkyl. Preferably, R¹⁶ and R¹⁷ are independently selected from the group consisting of hydrogen and methyl.

Alternatively, R¹⁶ and R¹⁷ together with the nitrogen atom to which they are attached form a 4- to 6-membered heterocyclyl ring which optionally comprises one additional heteroatom individually selected from N, O and S. Preferably, R¹⁶ and R¹⁷ together with the nitrogen atom to which they are attached form a 5- to 6-membered heterocyclyl ring which optionally comprises one additional heteroatom individually selected from N and O. More preferably, R¹⁶ and R¹⁷ together with the nitrogen atom to which they are attached form an pyrrolidyl, oxazolidinyl, imidazolidinyl, piperidyl, piperazinyl or morpholinyl group.

R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different. Preferably, R¹⁸ is selected from the group consisting of hydrogen, C₁-C₆alkyl, C₁-C₆haloalkyl, C₁-C₆alkoxy, —N(R⁶)₂ and phenyl. More preferably, R¹⁸ is selected from the group consisting of hydrogen, C₁-C₆alkyl and C₁-C₆haloalkyl. Further more preferably, R¹⁸ is selected from the group consisting of C₁-C₆alkyl and C₁-C₆haloalkyl. Most preferably, R¹⁸ is methyl or trifluoromethyl.

r is 0, 1 or 2. Preferably, r is 0 or 2.

It should be understood that compounds of formula (I) may exist/be manufactured in ‘procidal form’, wherein they comprise a group ‘G’. Such compounds are referred to herein as compounds of formula (I-V).

G is a group which may be removed in a plant by any appropriate mechanism including, but not limited to, metabolism and chemical degradation to give a compound of formula (I-I), (I-III) or (I-IV) wherein A contains an acidic proton, for example see the scheme below:

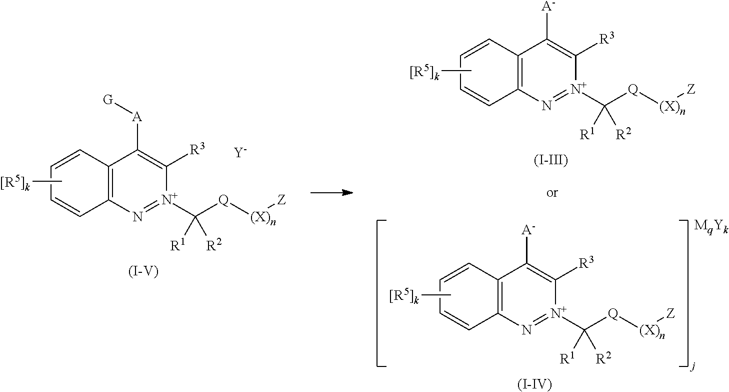

Whilst such G groups may be considered as ‘procidal’, and thus yield active herbicidal compounds once removed, compounds comprising such groups may also exhibit herbicidal activity in their own right. In such cases in a compound of formula (I-V), A-G may include but is not limited to, any one of (G1) to (G7) below and E indicates the point of attachment to the remaining part of a compound of formula (I):

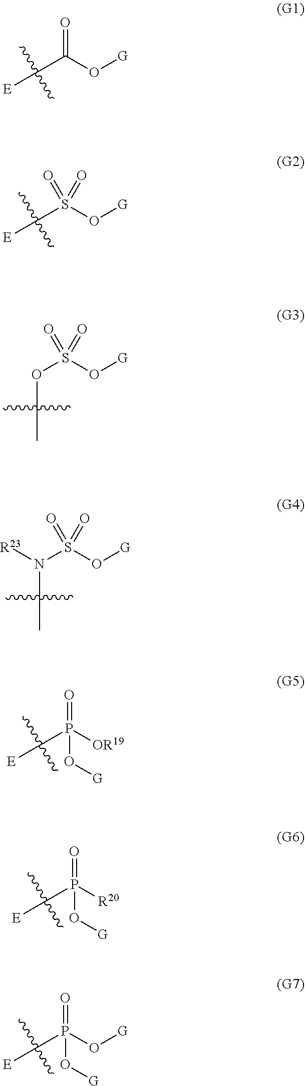

In embodiments where A-G is (G1) to (G7), G, R¹⁹, R²⁰, R²¹, R²² and R²³ are defined as follows:

G is C₁-C₆alkyl, C₂-C₆alkenyl, C₂-C₆alkynyl, —C(R²¹R²²)OC(O)R¹⁹, phenyl or phenyl-C₁-C₄alkyl-, wherein said phenyl moiety is optionally substituted by 1 to 5 substituents independently selected from halo, cyano, nitro, C₁-C₆alkyl, C₁-C₆haloalkyl or C₁-C₆alkoxy.

R¹⁹ is C₁-C₆alkyl or phenyl,

R²⁰ is hydroxy, C₁-C₆alkyl, C₁-C₆alkoxy or phenyl,

R²¹ is hydrogen or methyl,

R²² is hydrogen or methyl,

R²³ is hydrogen or C₁-C₆alkyl.

As stated above, all permissible combinations of substituents (and preference levels) are contemplated within the invention. For the avoidance of doubt, however, the invention explicitly encompasses the following embodiments.

In one set of embodiments, Z is Z1 and thus selected from the group consisting of hydrogen, —CH₂OH, and methoxy; k is 0; n is 0; m is 0; R¹ and R² independently hydrogen or methyl; R³ is selected from the group consisting of hydrogen, halogen and C₁-C₆alkyl, phenyl and thiazole, wherein said phenyl or thiazole is optionally substituted by 1 or 2 R⁹, which may be the same or different; each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy, and A is selected from the group consisting of —C(O)OR⁴¹⁰, —C(O)NHOR⁴¹¹, —C(O)NHR⁴²⁵, —S(O)₂NHR⁴²¹, —C(O)NHS(O)₂R⁴¹⁴, —C(O)NR⁴⁶(CR⁴⁶₂)_{q}C(O)OR⁴¹⁰, —C(O)NR⁴⁶S(O)₂(CR⁴⁶₂)_{q}C(O)OR⁴¹⁰, —(CR⁴⁶₂)_{q}C(O)OR⁴¹⁰, —(CR⁴⁶₂)_{q}P(O)(R⁴¹³)(OR⁴¹⁰), —OC(O)NHOR⁴¹¹, —O(CR⁴⁶₂)_{q}C(O)OR⁴¹⁰, —OC(O)NHCN, —O(CR⁴⁶₂)_{q}S(O)₂OR⁴¹⁰, —O(CR⁴⁶₂)_{q}P(O)(R⁴¹³)(OR⁴¹⁰), —S(O)₂OR⁴¹⁰, —S(CR⁴⁶₂)_{q}C(O)OR⁴¹⁰, —S(CR⁴⁶₂)_{q}S(O)₂OR⁴¹⁰, —P(O)(R⁴¹³)(OR⁴¹⁰), —P(O)H(OR⁴¹⁰), —OP(O)(R⁴¹³)(OR⁴¹⁰), and —NR⁴⁶P(O)(R⁴¹³)(OR⁴¹⁰), with the proviso that when A is —P(O)(R⁴¹³)(OR⁴¹⁰), R⁴¹³ is —OH, R⁴¹⁰ is C₁-C₆alkyl, and R¹ and R² are both hydrogen, then Z is CH₂OH or methoxy.

In a second set of embodiments, Z is Z1, k is 0; n is 0; m is 1; R¹ and R² independently hydrogen or methyl; Ria and R^{2b} are each independently of hydrogen, halogen, C₁-C₆alkyl, C₁-C₆fluoroalkyl, —OH, —NH₂ and —NHR⁷; R³ is selected from the group consisting of hydrogen, halogen and C₁-C₆alkyl, phenyl and thiazole, wherein said phenyl or thiazole is optionally substituted by 1 or 2 R⁹, which may be the same or different; each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy, and A is selected from the group consisting of —C(O)OR⁴¹⁰, —C(O)NHOR⁴¹¹, —C(O)NHR⁴²⁵, —S(O)₂NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶S(O)₂(CR⁴⁶₂)_{q}C(O)OR⁴¹⁰, —(CR⁴⁶₂)_{q}C(O)OR⁴¹⁰, —(CR⁴⁶₂)_{q}P(O)(R⁴¹³)(OR⁴¹⁰), —OC(O)NHOR⁴¹¹, —O(CR⁴⁶₂)_{q}C(O)OR⁴¹⁰, —OC(O)NHCN, —O(CR⁴⁶₂)_{q}S(O)₂OR⁴¹⁰, —O(CR⁴⁶₂)_{q}P(O)(R⁴¹³)(OR⁴¹⁰), S(O)₂OR⁴¹⁰, —S(CR⁴⁶₂)_{q}C(O)OR⁴¹⁰, —S(CR⁴⁶₂)_{q}S(O)₂OR⁴¹⁰, —P(O)(R⁴¹³)(OR⁴¹⁰), —P(O)H(OR⁴¹⁰), —OP(O)(R⁴¹³)(OR⁴¹⁰), and —NR⁴⁶P(O)(R⁴¹³)(OR⁴¹⁰).

In a third set of embodiments, Z is Z1, k is 0; n is 0; m is 3; R¹ and R² independently hydrogen or methyl; each R^{1a} and R^{2b} are each independently of hydrogen, or C₁-C₆alkyl; R³ is selected from the group consisting of hydrogen, halogen and C₁-C₆alkyl, phenyl and thiazole, wherein said phenyl or thiazole is optionally substituted by 1 or 2 R⁹, which may be the same or different; each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy, and A is selected from the group consisting of —C(O)OR⁴¹⁰, —C(O)NHOR⁴¹¹, —C(O)NHR⁴²⁵, —S(O)₂NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶S(O)₂(CR⁴⁶₂)_{q}C(O)OR⁴¹⁰, —(CR⁴⁶₂)_{q}C(O)OR⁴¹⁰, —(CR⁴⁶₂)_{q}P(O)(R⁴¹³)(OR⁴¹⁰), —OC(O)NHOR⁴¹¹, O(CR⁴⁶₂)_{q}C(O)OR⁴¹⁰, —OC(O)NHCN, —O(CR⁴⁶₂)_{q}S(O)₂OR⁴¹⁰, —O(CR⁴⁶₂)_{q}P(O)(R⁴¹³)(OR⁴¹⁰), —S(O)₂OR⁴¹⁰, —S(CR⁴⁶₂)_{q}C(O)OR⁴¹⁰, —S(CR⁴⁶₂)_{q}S(O)₂OR⁴¹⁰, —P(O)(R⁴¹³)(OR⁴¹⁰), —P(O)H(OR⁴¹⁰), —OP(O)(R⁴¹³)(OR⁴¹⁰), and —NR⁴⁶P(O)(R⁴¹³)(OR⁴¹⁰).

In a further set of embodiments Z is Z2 and is thus selected from the group consisting of C(O)OR¹⁰, —CHO, —C(O)NHOR¹¹, —C(O)NHCN, —OC(O)NHOR¹¹, —OC(O)NHCN, —NR⁶C(O)NHOR¹¹, —NR⁶C—(O)NHCN, —C(O)NHS(O)₂R¹², —OC(O)NHS(O)₂R¹², —NR⁶C(O)NHS(O)₂R¹², —S(O)₂OR¹⁰, —OS—(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole; m is 0 or 1; n is 0; R¹ and R² are independently hydrogen or methyl; each R^{1a} and each R^{2b} are independently hydrogen, halogen, methyl, ethyl, propyl or butyl; k is 0; R³ is selected from hydrogen, halogen, cyano, nitro, —S(O)ᵣR¹⁵, C₁-C₆alkyl, C₁-C₆halooalkyl, C₁-C₆haloalkoxy, C₁-C₆alkoxy, C₃-C₆cycloalkyl, —N(R⁶)₂, phenyl, a 5- or 6-membered heteroaryl comprising 1, 2, 3 or 4 heteroatoms individually selected from N, O and S, and a 4- to 6-membered heterocyclyl comprising 1, 2 or 3 heteroatoms individually selected from N, O and S, and wherein said phenyl, heteroaryl or heterocyclyl moieties are optionally substituted by 1 or 2 R⁹ substituents; each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy; and A is selected from the group consisting of —C(O)OR⁴¹⁰, —CHO, —C(O)R⁴²⁴, —C(O)NHOR⁴¹¹, —C(O)NHCN, —C(O)NHR⁴²⁵, —S(O)₂NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶(CR⁴⁶₂)_{q}—S(O)₂OR⁴¹⁰, —C(O)NR⁴⁶(CR⁴⁶₂)_{q}P(O)(R⁴¹³)OR⁴¹⁰, —C(O)NR⁴⁶S(O)₂(CR⁴⁶₂)_{q}C(O)OR⁴¹⁰, —(CR⁴⁶₂)_{q}C(O)OR⁴¹⁰, —(CR⁴⁶₂)_{q}S(O)₂OR⁴¹⁰, —(CR⁴⁶₂)_{q}P(O)(R⁴¹³)(OR⁴¹⁰), —(CR⁴⁶₂)_{q}P(O)(R⁴¹³)(OR⁴¹⁰), —OC(O)NHOR⁴¹¹, —O(CR⁴⁶₂)_{q}C(O)OR⁴¹⁰, —OC(O)NHCN, —O(CR⁴⁶₂)_{q}S(O)₂OR⁴¹⁰, O(CR⁴⁶₂)_{q}P(O)(R⁴¹³)(OR⁴¹⁰), —NR⁴⁶C(O)NHOR⁴¹¹, —NR⁴⁶C(O)NHCN, —C(O)NHS(O)₂R⁴¹², —OC(O)NHS(O)₂R⁴¹², —NR⁴⁶C(O)NHS(O)₂R⁴¹², —S(O)₂OR⁴¹⁰, —OS(O)₂OR⁴¹⁰, —NR⁴⁶S(O)₂OR⁴¹⁰, —NR⁴⁶S(O)OR⁴¹⁰, —NHS(O)₂R⁴¹⁴, —S(O)₂OR⁴¹⁰, —S(CR⁴⁶₂)_{q}C(O)OR⁴¹⁰, S(CR⁴⁶₂)_{q}S(O)₂OR⁴¹⁰, —S(CR⁴⁶₂)_{q}P(O)(R⁴¹³)(OR⁴¹⁰), —OS(O)OR⁴¹⁰, —S(O)₂NHCN, —S(O)₂NHC(O)R⁴¹¹, —S(O)₂NHS(O)₂R⁴¹², —OS(O)₂NHCN, —OS(O)₂NHS(O)₂R⁴¹², —OS(O)₂NHC(O)R⁴¹¹, —NR⁴⁶S(O)₂NHCN, —NR⁴⁶S(O)₂NHC(O)R⁴¹¹, —N(OH)C(O)R⁴¹⁵, —ONHC(O)R⁴¹⁵, —NR⁴⁶S(O)₂NHS(O)₂R⁴¹², —P(O)(R⁴¹³)(OR⁴¹⁰), —P(O)H(OR⁴¹⁰), —OP(O)(R⁴¹³)(OR⁴¹⁰), —NR⁴⁶P(O)(R⁴¹³)(OR⁴¹⁰) and tetrazole.

In this set of embodiments it is preferred that Z is selected from the group consisting of —C(O)OR¹⁰, —CH₂OH, —C(O)NHOR¹¹, —C(O)NHS(O)₂R¹², —S(O)₂₀R¹⁰, —OS(O)₂OR¹⁰, —NR'S(O)₂OR¹⁰, —NHS(O)₂R¹⁴, —S(O)OR¹⁰, —P(O)(R¹³)(OR¹⁰) and tetrazole, and even more preferred that Z is selected from the group consisting of C(O)OR¹⁰, —C(O)NHS(O)₂R¹²—S(O)₂OR¹⁰, —OS(O)₂OR¹⁰.

In this set of embodiments it is also preferred that A is selected from the group consisting of —C(O)OR⁴¹⁰, —C(O)NHOR⁴¹¹, —C(O)NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶S(O)₂(CR⁴⁶₂)_{q}C(O)—OR⁴¹⁰, —(CR⁴⁶₂)_{q}C(O)OR⁴¹⁰, —(CR⁴⁶₂)_{q}P(O)(R⁴¹³)(OR⁴¹⁰), —S(O)₂—OR⁴¹⁰, —S(CR⁴⁶₂)_{q}C(O)OR⁴¹⁰, —O(CR⁴⁶₂)_{q}C(O)OR⁴¹⁰, and —P(O)(R⁴¹³)(OR⁴¹⁰), and even more preferred that A is selected from the group consisting of —C(O)OR⁴¹⁰, —C(O)NHS(O)₂R⁴¹⁴, —S(O)₂—OR¹⁰, and —P(O)(R⁴¹³)(OR⁴¹⁰).

The compounds in Tables 1 to 4 below illustrate the compounds of the invention. The skilled person would understand that the compounds of formula (I) may exist as an agronomically acceptable salt, a zwitterion or an agronomically acceptable salt of a zwitterion as described hereinbefore.

TABLE 1
This table discloses 40 specific compounds of the formula (T-1):
(T-1)

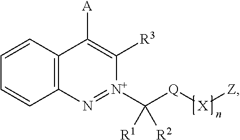

wherein R³, A, Z, m and Q are as defined in the table below,
R¹ and R² are hydrogen and n is 0.

| Compound | | | | | |
|---|---|---|---|---|---|
| number | R³ | A | Z | m | Q |
| 1.001 | H | —P(O)(OH)(Me) | —H | 0 | — |
| 1.002 | H | —P(O)(OH)(Me) | —H | 1 | —CH₂ |
| 1.003 | H | —P(O)(OH)(Me) | —CH₂OH | 0 | — |
| 1.004 | H | —P(O)(OH)(Et) | —H | 0 | — |
| 1.005 | H | —P(O)(OH)(Et) | —H | 1 | —CH₂ |
| 1.006 | H | —P(O)(OH)(Et) | —CH₂OH | 0 | — |
| 1.007 | H | —P(O)(OH)(Pr) | —H | 0 | — |
| 1.008 | H | —P(O)(OH)(Pr) | —H | 1 | —CH₂ |
| 1.009 | H | —P(O)(OH)(Pr) | —CH₂OH | 0 | — |
| 1.010 | H | —P(O)(OH)(iPr) | —H | 0 | — |
| 1.011 | H | —P(O)(OH)(iPr) | —H | 1 | —CH₂ |
| 1.012 | H | —P(O)(OH)(iPr) | —CH₂OH | 0 | — |
| 1.013 | H | —P(O)(OH)(Bu) | —H | 0 | — |
| 1.014 | H | —P(O)(OH)(Bu) | —H | 1 | —CH₂ |
| 1.015 | H | —P(O)(OH)(Bu) | —CH₂OH | 0 | — |
| 1.016 | H | —C(O)NHSO₂Me | —H | 0 | — |
| 1.017 | H | —C(O)NHSO₂Me | —H | 1 | —CH₂ |
| 1.018 | H | —C(O)NHSO₂Me | —CH₂OH | 0 | — |
| 1.019 | H | —C(O)NHSO₂Et | —H | 0 | — |
| 1.020 | H | —C(O)NHSO₂Et | —H | 1 | —CH₂ |
| 1.021 | H | —C(O)NHSO₂Et | —CH₂OH | 0 | — |
| 1.022 | H | —C(O)OH | —H | 0 | — |
| 1.023 | H | —C(O)OH | —H | 1 | —CH₂ |
| 1.024 | H | —C(O)OH | —CH₂OH | 0 | — |
| 1.025 | H | —P(O)(OH)(OMe) | —H | 1 | —CH₂ |
| 1.026 | H | —P(O)(OH)(OMe) | —CH₂OH | 0 | — |
| 1.027 | H | —P(O)(OH)(OEt) | —H | 1 | —CH₂ |
| 1.028 | H | —P(O)(OH)(OEt) | —CH₂OH | 0 | — |
| 1.029 | H | —P(O)(OH)(OPr) | —H | 1 | —CH₂ |
| 1.030 | H | —P(O)(OH)(OPr) | —CH₂OH | 0 | — |
| 1.031 | H | —P(O)(OH)(OiPr) | —H | 1 | —CH₂ |
| 1.032 | H | —P(O)(OH)(OiPr) | —CH₂OH | 0 | — |
| 1.033 | H | —P(O)(OH)(OBu) | —H | 1 | —CH₂ |
| 1.034 | H | —P(O)(OH)(OBu) | —CH₂OH | 0 | — |
| 1.035 | H | —P(O)(OH)(O-allyl) | —H | 1 | —CH₂ |
| 1.036 | H | —P(O)(OH)(O-allyl) | —CH₂OH | 0 | — |
| 1.037 | H | —P(O)(OH)(O-propargyl) | —H | 1 | —CH₂ |
| 1.038 | H | —P(O)(OH)(O-propargyl) | —CH₂OH | 0 | — |
| 1.039 | H | —P(O)(OH)(OCH₂CF₃) | —H | 1 | —CH₂ |
| 1.040 | H | —P(O)(OH)(OCH₂CF₃) | —CH₂OH | 0 | — |

TABLE 2
This table discloses 40 specific compounds of the formula (T-2)

(T-2)

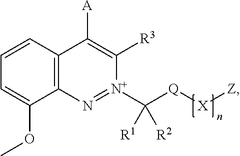

wherein R³, A, Z, m and Q are as defined in Table 1 above,
R¹ and R² are hydrogen and n is 0.

wherein R³, A, Z, m and Q are as defined in Table 1 above, R¹ and R² are hydrogen and n is 0.

TABLE 3
This table discloses 40 specific compounds of the formula (T-3):

(T-3)

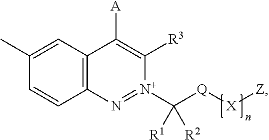

wherein R³, A, Z, m and Q are as defined in Table above,
R¹ and R² are hydrogen and n is 0.

wherein R³, A, Z, m and Q are as defined in Table 1 above, R¹ and R² are hydrogen and n is 0.

TABLE 4
This table discloses 40 specific compounds of the formula (T-4):

(T-4)

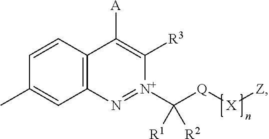

wherein R³, A, Z, m and Q are as defined in Table 1,
R¹ and R² are hydrogen and n is 0.

wherein R³, A, Z, m and Q are as defined in Table 1, R¹ and R² are hydrogen and n is O.

The compounds of the present invention may be prepared according to the following schemes in which the substituents R¹, R², R^{1a}, R^{2b}, R³, R⁵, R⁶, R⁷, R^{7a}, R^{7b}, R^{7c}, R⁹, R¹⁰, R¹¹, R¹², R¹³, R¹⁴, R¹⁵, R^{15a}, R¹⁶, R¹⁷, R¹⁸, R⁴⁶, R⁴⁹, R⁴¹⁰, R⁴¹¹, R⁴¹², R⁴¹³, R⁴¹⁴, R⁴¹⁵, R⁴¹⁸, R⁴²⁰, R⁴²⁴, R⁴²⁵, A, Q, X, and Z, and integers k, m, n, q and r, are as defined hereinbefore unless explicitly stated otherwise.

The compounds of formula (I) may be prepared by the alkylation of compounds of formula (X), wherein R³, R⁵, k and A are as defined for compounds of formula (I), with a suitable alkylating agent of formula (W), wherein R¹, R², Q, X, n, and Z are as defined for compounds of formula (I) and LG is a suitable leaving group, for example halide or pseudohalide such as triflate, mesylate or tosylate, in a suitable solvent at a suitable temperature, as described in reaction scheme 1. Example conditions include stirring a compound of formula (X) with an alkylating agent of formula (W) in a solvent, or mixture of solvents, such as acetone, dichloromethane, dichloroethane, N,N-dimethylformamide, acetonitrile, 1,4-dioxane, water, acetic acid or trifluoroacetic acid at a temperature between −78° C. and 150° C. Alkylating agents of formula (W) are commercially available or are known in the literature and may include, but are not limited to, iodomethane, bromomethane, chloromethane, dimethylsulfate, iodoethane, bromoethane, chloroethane, diethylsulfate, 2-methoxyethyl trifluoromethanesulfonate, 2-bromoethyl methyl ether, 2-iodoethyl methyl ether, benzyl bromide, benzyl chloride, benzyl iodide, 2-bromoethanol, 2-iodoethanol, 2,2-difluoroethyl trifluoromethanesulfonate, 2-bromoethylamine hydrobromide, bromoacetic acid, methyl bromoacetate, 3-bromopropionoic acid, methyl 3-bromopropionate, 2-bromo-N-methoxyacetamide, sodium 2-bromoethanesulphonate, 2,2-dimethylpropyl 2-(trifluoromethylsulfonyloxy)ethanesulfonate, 2-bromo-N-methanesulfonylacetamide, 3-bromo-N-methanesulfonylpropanamide, dimethoxyphosphorylmethyl trifluoromethanesulfonate, dimethyl 3-bromopropylphosphonate, 3-chloro-2,2-dimethyl-propanoic acid and diethyl 2-bromoethylphosphonate. Such alkylating agents and related compounds are either known in the literature or may be prepared by known literature methods. Compounds of formula (I) which may be described as esters of N-alkyl acids, which include, but are not limited to, esters of carboxylic acids, phosphonic acids, phosphinic acids, sulfonic acids and sulfinic acids, may optionally be subsequently partially or fully hydrolysed by treament with a suitable reagent, for example, aqueous hydrochloric acid or trimethylsilyl bromide, in a suitable solvent at a suitable temperature between 0° C. and 100° C.

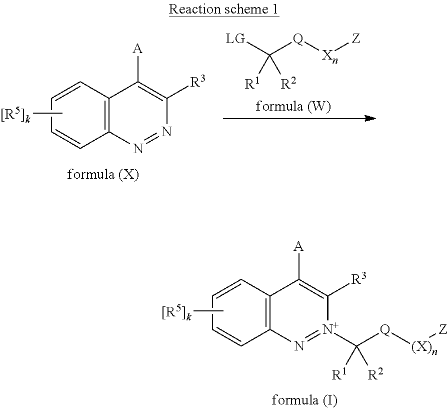

Additionally, compounds of formula (I) may be prepared by reacting compounds of formula (X), wherein wherein R³, R⁵, k and A are as defined for compounds of formula (I), with a suitably activated electrophilic alkene of formula (B), wherein Z is —S(O)₂OR¹⁰, —P(O)(R¹³)(OR¹⁰), C(O)NR¹⁶R¹⁷, S(O)₂NR¹⁶R¹⁷, nitro, cyano, S(O)₂R¹⁵, C(O)R¹⁵ or —C(O)OR¹⁰ and R¹, R². R^{1a}, R¹⁰, R¹³, R¹⁵, R¹⁶ and R¹⁷ are as defined for compounds of formula (I), in a suitable solvent at a suitable temperature.

Compounds of formula (B) are known in the literature, or may be prepared by known methods. Example reagents include, but are not limited to, acrylic acid, methacrylic acid, crotonic acid, 3,3-dimethylacrylic acid, methyl acrylate, ethene sulfonic acid, isopropyl ethylenesulfonate, 2,2-dimethylpropyl ethenesulfonate and dimethyl vinylphosphonate. The direct products of these reactions, which may be described as esters of N-alkyl acids, which include, but are not limited to, esters of carboxylic acids, phosphonic acids, phosphinic acids, sulfonic acids and sulfinic acids, may optionally be subsequently partially or fully hydrolysed by treatment with a suitable reagent in a suitable solvent at a suitable temperature, as described in reaction scheme 2.

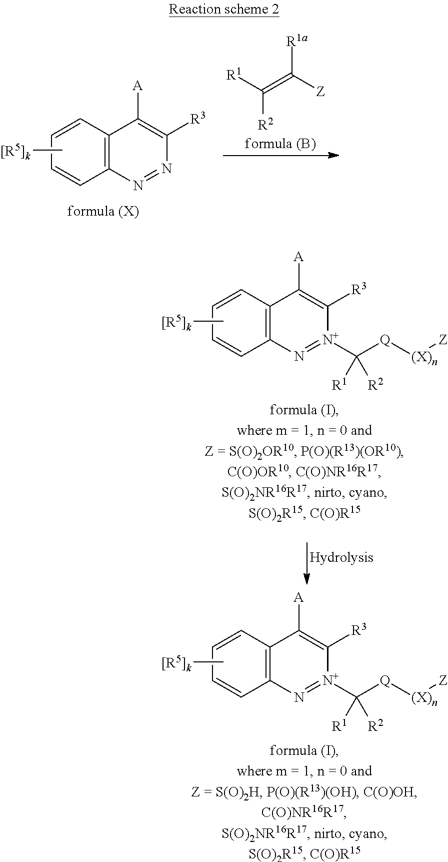

In a related reaction compounds of formula (I), wherein Q is C(R₁ₐR_{2b}), m is 1, 2 or 3, n=0 and Z is —S(O)₂OH, —OS(O)₂OH or —NR⁶S(O)₂OH, may be prepared by the reaction of compounds of formula (X), wherein R³, R⁵, k and A are as defined for compounds of formula (I), with a cyclic alkylating agent of formula (E), (F) or (AF), wherein Y^{a} is C(R^{1a}R^{2b}), O or NR⁶ and R¹, R², R^{1a} and R^{2b} are as defined for compounds of formula (I), in a suitable solvent at a suitable temperature, as described in reaction scheme 3.

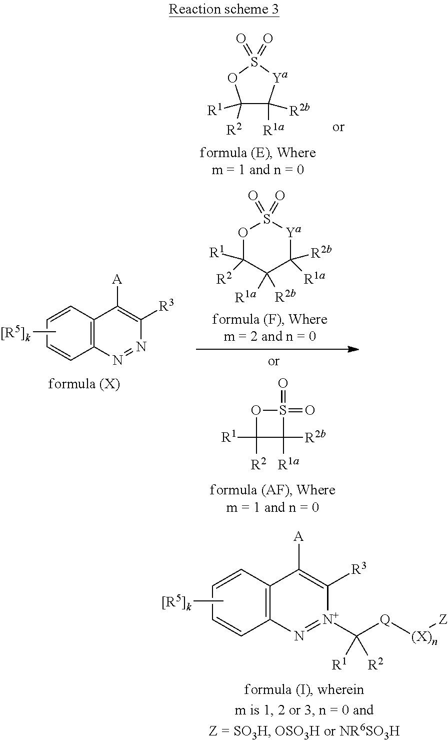

Suitable solvents and suitable temperatures are as previously described. An alkylating agent of formula (E) or (F) may include, but is not limited to, 1,3-propanesultone, 1,4-butanesultone, ethylenesulfate, 1,3-propylene sulfate and 1,2,3-oxathiazolidine 2,2-dioxide. Such alkylating agents and related compounds are either known in the literature or may be prepared by known literature methods.

A compound of formula (I), wherein m is 0, n is 0 and Z is —S(O)₂OH, may be prepared from a compound of formula (I), wherein m is 0, n is 0 and Z is C(O)OR¹⁰, by treatment with trimethylsilylchlorosulfonate in a suitable solvent at a suitable temperature, as described in reaction scheme 4. Preferred conditions include heating the carboxylate precursor in neat trimethylsilylchlorosulfonate at a temperature between 25° C. and 150° C.

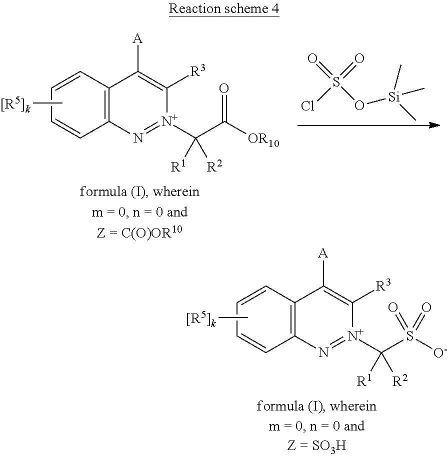

Furthermore, compounds of formula (I) may be prepared by reacting compounds of formula (X), wherein R³, R⁵, k and A are as defined for compounds of formula (I), with a suitable alcohol of formula (WW), wherein R¹, R², Q, X, n and Z are as defined for compounds of formula (I), under Mitsunobu-type conditions such as those reported by Petit et al, Tet. Lett. 2008, 49 (22), 3663. Suitable phosphines include triphenylphosphine, suitable azodicarboxylates include diisopropylazodicarboxylate and suitable acids include fluoroboric acid, triflic acid and bis(trifluoromethylsulfonyl)amine, as described in reaction scheme 5. Such alcohols are either known in the literature or may be prepared by known literature methods.

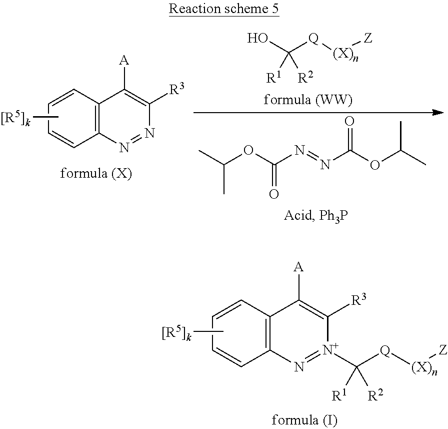

Compounds of formula (X) are known in the literature, or may be prepared by known methods. See for example Chen, X., Zheng, G., Song, G., Li, X., Adv. Synth. Catal., 2018, 360(15), 2836, Ponte, J. R. et al, U.S. Pat. No. 4,666,499, Armarego, W. L. F., Batterham, T. J., Schofield, K., Theobald, R. S., Journal of the Chemical Society C: Organic, 1966, (6), 1433, Barber, H. J., Lunt, E., Journal of the Chemical Society C: Organic, 1968, (9), 1156, Hayashi, E., Watanabe, T., Yakugaku Zasshi, 1968, 88(6), 742, Nagarajan, K., Shah, R. K., Shenoy, S. J., Indian J. Chem., Sect B, 1986, 25B(7), 697, Mizuno, Y., Adachi, K., Ikeda, K., Pharmaceutical Bulletin, 1954, 2, 225, Somei, M., Kurizuka, Y., Chem. Lett., 1979, (2), 127 and Denes et al, EP 212726.

Compounds of formula (X), wherein A is —C(O)R⁴²⁴, —C(O)NHOR⁴¹¹, —C(O)NHCN, —C(O)NHR⁴²⁵, —C(O)NR⁴⁶(CR⁴⁶₂)_{q}C(O)OR⁴¹⁰, —C(O)NR⁴⁶(CR⁴⁶₂)_{q}S(O)₂OR⁴¹⁰, —C(O)NR⁴⁶(CR⁴⁶₂)_{q}P(O)(R⁴¹³)OR⁴¹⁰, and R³, R⁵, R⁴⁶, R⁴¹⁰, R⁴¹¹, R⁴¹³, R⁴²⁴, R⁴²⁵, k and q are as defined for a compound of formula (I), may be prepared from a compound of formula (J), wherein T is a halogen or T is an ester or activated ester, for example—OC₁-C₆alkyl, pentafluorophenol, p-nitrophenol, 2,4,6-trichlorophenol, —OC(O)R or —OS(O)₂R′″ and R′″ is, for example, C₁-C₆alkyl, C₁-C₆haloalkyl or optionally substituted phenyl, by reacting with an amine, for example but not limited to, of formula —R⁴²⁴, NH₂OR⁴¹¹, NH₂CN, NH₂R⁴²⁵, NHR⁴⁶(CR⁴⁶₂)_{q}C(O)OR⁴¹⁰, NHR⁴⁶(CR⁴⁶₂)_{q}S(O)₂OR⁴¹⁰ or NHR⁴⁶(CR⁴⁶₂)_{q}P(O)(R⁴¹³)OR⁴¹⁰, in a suitable solvent or mixture of solvents, optionally in the presence of a suitable base at a suitable temperature between −78° C. and 200° C., as described in reaction scheme 6. Suitable bases include, but are not limited to, triethylamine, pyridine, N,N-diisopropylethylamine, an alkali metal carbonate, such as sodium carbonate, potassium carbonate or cesium carbonate, or an alkali metal alkoxide, such as sodium methoxide. Suitable solvents include, but are not limited to, dichloromethane, N,N-dimethylformamide, THE or toluene. Compounds of formula (J) are either known in the literature or may be prepared by known literature methods or may be commercially available.

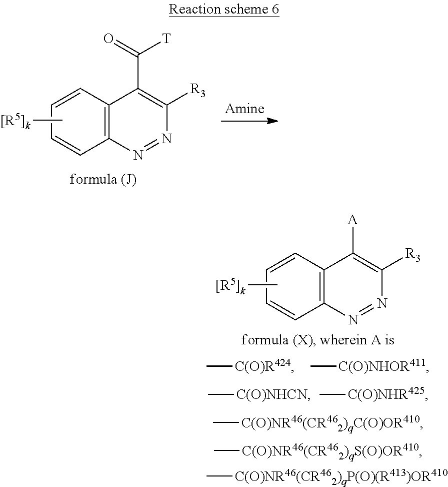

Compounds of formula (X), wherein A is —C(O)R⁴²⁴, —C(O)NHOR⁴¹¹, —C(O)NHCN, —C(O)NHR⁴²⁵, —C(O)NR⁴⁶(CR⁴⁶₂)_{q}C(O)OR⁴¹⁰, —C(O)NR⁴⁶(CR⁴⁶₂)_{q}S(O)₂OR⁴¹⁰, —C(O)NR⁴⁶(CR⁴⁶₂)_{q}P(O)(R⁴¹³)OR⁴¹⁰, wherein R³, R⁵, R⁴⁶, R⁴¹⁰, R⁴¹¹, R⁴¹³, R⁴²⁴, R⁴²⁵, k and q are as defined previously, may be prepared from a carboxylic acid of formula (L) by classical amide bond forming reactions which are very well known in the literature, as described in reaction scheme 7. Such reactions include, but are not limited to, reacting a carboxylic acid of formula (L) with an amine, for example, of formula —R⁴²⁴, NH₂OR⁴¹¹, NH₂CN, NH₂R⁴²⁵, NHR⁴⁶(CR⁴⁶₂)_{q}C(O)OR⁴¹⁰, NHR⁴⁶(CR⁴⁶₂)_{q}S(O)₂OR⁴¹⁰, NH₂S(O)₂R⁴¹² or NHR⁴⁶(CR⁴⁶₂)_{q}P(O)(R⁴¹³)OR⁴¹⁰, wherein R⁴⁶, R⁴¹⁰, R⁴¹¹, R⁴¹², R⁴¹³, R⁴²⁴, R⁴²⁵ and q are as defined for compounds of formula (I), in the presence of a suitable coupling agent in a suitable solvent or mixture of solvents, at a suitable temperature between −78° C. and 200° C., and optionally in the presence of a suitable base. Suitable coupling reagents include, but are not limited to, a carbodiimide, for example dicyclohexylcarbodiimide or 1-ethyl-3-[3-dimethylaminopropyl]carbodiimide hydrochloride, a phosphonic anhydride, for example 2,4,6-tripropyl-1,3,5,2,4,6-trioxatriphosphorinane-2,4,6-trioxide, or a phosphonium salt, for example benzotriazol-1-yloxy(tripyrrolidin-1-yl)phosphonium hexafluorophosphate. Suitable solvents include, but are not limited to, dichloromethane, N,N-dimethylformamide, THE or toluene, and suitable bases include, but are not limited to, triethylamine, pyridine and N,N-diisopropylethylamine. Compounds of formula (L) are either known in the literature or may be prepared by known literature methods or may be commercially available.

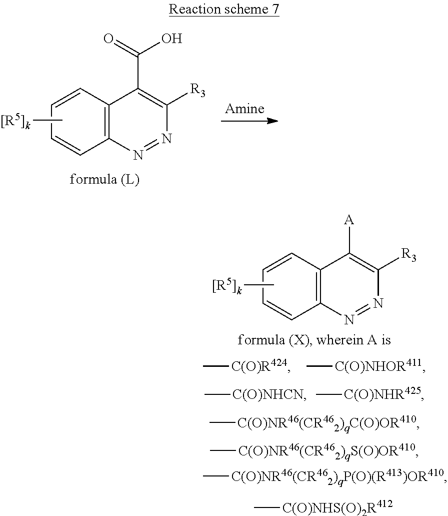

A compound of formula (X), wherein A is —P(O)(R⁴¹³)(OR⁴¹⁰) or —P(O)H(OR⁴¹⁰) and R³, R⁵, R⁴¹⁰, R⁴¹³ and k are as defined previously, may be prepared from a compound of formula (ZZ), wherein LG is a leaving group, for example, halide or pseudohalide, such as triflate, mesylate or tosylate, as described in reaction scheme 8. Example conditions include reacting a compound of formula (X) with a reagent of formula P(R¹³)(OR¹⁰)₂ or P(O)(R¹³)(OR¹⁰)H in the presence of an appropriate transition metal catalyst, ligand and base, in an appropriate solvent and at an appropriate temperature. See, for example, Keglevich, G., Gruen, A., Boelcskei, A., Drahos, L., Kraszni, M., Balogh, G. T., Heteroatom Chemistry, 23(6), 2012, 574, Fang, C., Chen, Z., Liu, X., Yang, Y., Deng, M., Weng, L., Jia, Y., Zhou, Y., Inorganica Chimica Acta, 362(7), 2009, 2101 and Hynek, J., Brazda, P., Rohlicek, J., Londesborough, M. G. S., Demel, J., Angewandte Chemie, International Edition, 57(18), 2018, 5016.

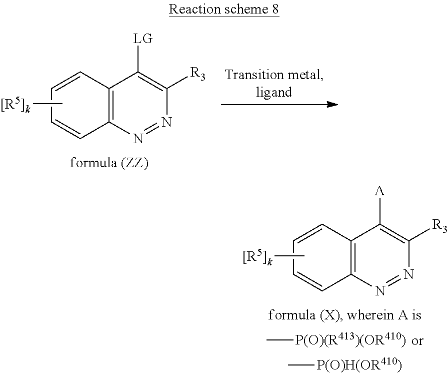

In an alternative approach a compound of formula (X) may be prepared by nucleophilic displacement on a compound of formula (ZZ), wherein LG includes, but is not limited to, halide or pseudohalide, such as triflate, mesylate or tosylate, or a compound of formula (Y), as described in reaction scheme 9. Similar reactions are known in the literature, see for example Gardner, G.; Steffens, J. J.; Grayson, B. T.; Kleier, D. A. J. Agric. Food. Chem., 1992, 318-321, and Miyashita, A.; Suzuki, Y.; Iwamoto, K.; Oishi, E.; Higashino, T. Heterocycles, 1998, 49, 405). Compounds of formula (Y) are known in the literature, for example, Kleier, D. A. J. Agric. Food. Chem., 1992, 318-321, Barlin, G. B.; Brown, W. V. J. Chem. Soc (C), 1969, 921-923 and Klatt, T. et al Org. Lett. 2014, 16, 1232-1235.

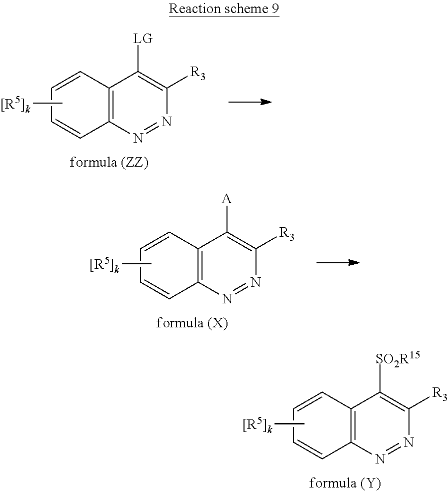

A compound of formula (ZZ), wherein R³, R⁵ and k are as defined for compounds of formula (I) and LG is a halide, may be prepared from a 4-hydroxycinnoline of formula (AZ) by treatment with known halogenating agents, such as phosphoryl halide, in a suitable solvent at a suitable temperature, as described in reaction scheme 10. See, for example, Ruchelman, A. L. et al Bioorg. Med. Chem., 2004, 12(4), 795-806).

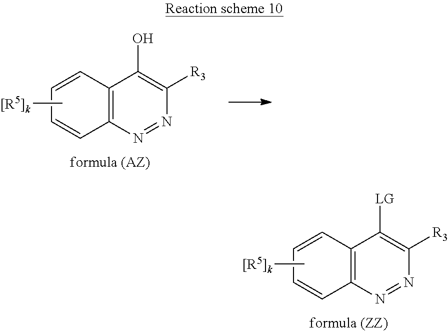

Hydroxycinnolines of formula (AZ) may be prepared by the diazotisation of an optionally substituted 2-aminoarylketone of formula (L) with either an inorganic nitrite or alkyl nitrite in the presence of acid in a suitable solvent at a suitable temperature, for example, Borsche, W.; Herbert, A. Liebigs Ann. Chem., 1941, 546, 293, and Koelsch, C. F. J. Org. Chem., 1943, 8, 295, as described in reaction scheme 11. Compounds of formula (L) are known in the literature or may be prepared by known methods, for example, Jana, S. et al Org. Biomol. Chem., 2015, 13(31), 8411-8415.

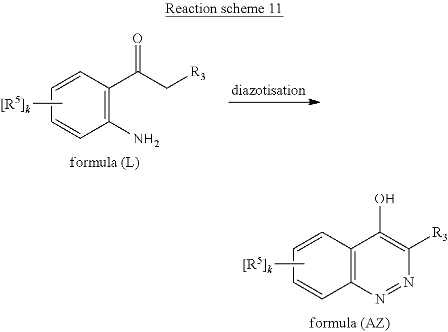

In an alternative approach a compound of formula (AZ) may be prepared by a sequence starting with the oxidation of a 2-haloacetophenone of formula (R), wherein R³, R⁵ and k are as defined for a compound of formula (I) and Hal is a halide, using a suitable oxidizing agent in a suitable solvent at a suitable temperature, for example selenium dioxide in 1,4-dioxane at a temperature between 25° C. to 100° C. Compounds of formula (S), wherein R³, R⁵ and k are as defined for a compound of formula (I), may be condensed with an optionally protected hydrazine, wherein PG is either hydrogen or a suitable protecting group, such as tert-butyl carbazate, to afford a hydrazone of formula (T), wherein R³, R⁵ and k are as defined for a compound of formula (I), preferably in the presence of an acid catalyst in a suitable solvent at a suitable temperature. Cyclisation of a compound of formula (T) to a compound of formula (AZ) may be achieved by treatment with a suitable base in a suitable solvent at a suitable temperature, for example potassium carbonate in N,N-dimethylformamide at a temperature between 25° C. and 150° C. This sequence of reactions is described in reaction scheme 12.

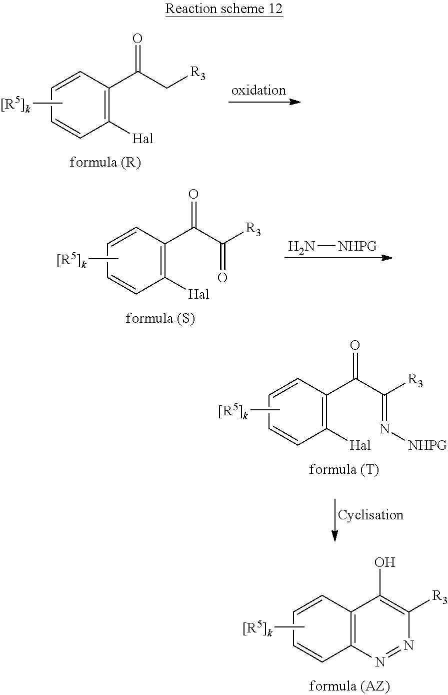

Compounds of formula (R) are known in the literature or may be prepared by known methods (for example Ruan, J. et al J. Am. Chem. Soc., 2010, 132(46), 16689-16699; 2010 and Ridge, D. N. et al J. Med. Chem., 1979, 22(11), 1385-1389).

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydro-furfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecyl-benzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid(methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The composition of the present may further comprise at least one additional pesticide. For example, the compounds according to the invention can also be used in combination with other herbicides or plant growth regulators. In a preferred embodiment the additional pesticide is a herbicide and/or herbicide safener.

Thus, compounds of formula (I) can be used in combination with one or more other herbicides to provide various herbicidal mixtures. Specific examples of such mixtures include (wherein “I” represents a compound of formula (I)): —I+acetochlor; I+acifluorfen (including acifluorfen-sodium); I+aclonifen; I+alachlor; I+alloxydim; I+ametryn; I+amicarbazone; I+amidosulfuron; I+aminocyclopyrachlor; I+aminopyralid; I+amitrole; I+asulam; I+atrazine; I+bensulfuron (including bensulfuron-methyl); I+bentazone; I+bicyclopyrone; I+bilanafos; I+bifenox; I+bispyribac-sodium; I+bixlozone; I+bromacil; I+bromoxynil; I+butachlor; I+butafenacil; I+cafenstrole; I+carfentrazone (including carfentrazone-ethyl); cloransulam (including cloransulam-methyl); I+chlorimuron (including chlorimuron-ethyl); I+chlorotoluron; I+cinosulfuron; I+chlorsulfuron; I+cinmethylin; I+clacyfos; I+clethodim; I+clodinafop (including clodinafop-propargyl); I+clomazone; I+clopyralid; I+cyclopyranil; I+cyclopyrimorate; I+cyclosulfamuron; I+cyhalofop (including cyhalofop-butyl); I+2,4-D (including the choline salt and 2-ethylhexyl ester thereof); I+2,4-DB; I+daimuron; I+desmedipham; I+dicamba (including the aluminum, aminopropyl, bis-aminopropylmethyl, choline, dichloroprop, diglycolamine, dimethylamine, dimethylammonium, potassium and sodium salts thereof); I+diclofop-methyl; I+diclosulam; I+diflufenican; I+difenzoquat; I+diflufenican; I+diflufenzopyr; I+dimethachlor; I+dimethenamid-P; I+diquat dibromide; I+diuron; I+esprocarb; I+ethalfluralin; I+ethofumesate; I+fenoxaprop (including fenoxaprop-P-ethyl); I+fenoxasulfone; I+fenquinotrione; I+fentrazamide; I+flazasulfuron; I+florasulam; I+florpyrauxifen; I+fluazifop (including fluazifop-P-butyl); I+flucarbazone (including flucarbazone-sodium; I+flufenacet; I+flumetralin; I+flumetsulam; I+flumioxazin; I+flupyrsulfuron (including flupyrsulfuron-methyl-sodium; I+fluroxypyr (including fluroxypyr-meptyl; I+fluthiacet-methyl; I+fomesafen; I+foramsulfuron; I+glufosinate (including the ammonium salt thereof); I+glyphosate (including the diammonium, isopropylammonium and potassium salts thereof); I+halauxifen (including halauxifen-methyl); I+halosulfuron-methyl; I+haloxyfop (including haloxyfop-methyl); I+hexazinone; I+hydantocidin; I+imazamox; I+imazapic; I+imazapyr; I+imazaquin; I+imazethapyr; I+indaziflam; I+iodosulfuron (including iodosulfuron-methyl-sodium); I+iofensulfuron; I+iofensulfuron-sodium; I+ioxynil; I+ipfencarbazone; I+isoproturon; I+isoxaben; I+isoxaflutole; I+lactofen; I+lancotrione; I+linuron; I+MCPA; I+MCPB; I+mecoprop-P; I+mefenacet; I+mesosulfuron; I+mesosulfuron-methyl; I+mesotrione; I+metamitron; I+metazachlor; I+methiozolin; I+metobromuron; I+metolachlor; I+metosulam; I+metoxuron; I+metribuzin; I+metsulfuron; I+molinate; I+napropamide; I+nicosulfuron; I+norflurazon; I+orthosulfamuron; I+oxadiargyl; I+oxadiazon; I+oxasulfuron; I+oxyfluorfen; I+paraquat dichloride; I+pendimethalin; I+penoxsulam; I+phenmedipham; I+picloram; I+picolinafen; I+pinoxaden; I+pretilachlor; I+primisulfuron-methyl; I+prodiamine; I+prometryn; I+propachlor; I+propanil; I+propaquizafop; I+propham; I+propyrisulfuron, I+propyzamide; I+prosulfocarb; I+prosulfuron; I+pyraclonil; I+pyraflufen (including pyraflufen-ethyl): I+pyrasulfotole; I+pyrazolynate, I+pyrazosulfuron-ethyl; I+pyribenzoxim; I+pyridate; I+pyriftalid; I+pyrimisulfan, I+pyrithiobac-sodium; I+pyroxasulfone; I+pyroxsulam; I+quinclorac; I+quinmerac; I+quizalofop (including quizalofop-P-ethyl and quizalofop-P-tefuryl,; I+rimsulfuron; I+saflufenacil; I+sethoxydim; I+simazine; I+S-metolachlor; I+sulcotrione; I+sulfentrazone; I+sulfosulfuron; I+tebuthiuron; I+tefuryltrione; I+tembotrione; I+terbuthylazine; I+terbutryn; I+thiencarbazone; I+thifensulfuron; I+tiafenacil; I+tolpyralate; I+topramezone; I+tralkoxydim; I+triafamone; I+triallate; I+triasulfuron; I+tribenuron (including tribenuron-methyl); I+triclopyr; I+trifloxysulfuron (including trifloxysulfuron-sodium); I+trifludimoxazin; I+trifluralin; I+triflusulfuron; I+tritosulfuron; I+4-hydroxy-1-methoxy-5-methyl-3-[4-(trifluoromethyl)-2-pyridyl]imidazolidin-2-one; I+4-hydroxy-1,5-dimethyl-3-[4-(trifluoromethyl)-2-pyridyl]imidazolidin-2-one; I+5-ethoxy-4-hydroxy-1-methyl-3-[4-(trifluoromethyl)-2-pyridyl]imidazolidin-2-one; I+4-hydroxy-1-methyl-3-[4-(trifluoromethyl)-2-pyridyl]imidazolidin-2-one; I+4-hydroxy-1,5-dimethyl-3-[1-methyl-5-(trifluoromethyl)pyrazol-3-yl]imidazolidin-2-one; I+(4R)1-(5-tert-butylisoxazol-3-yl)-4-ethoxy-5-hydroxy-3-methyl-imidazolidin-2-one; I+3-[2-(3,4-dimethoxyphenyl)-6-methyl-3-oxo-pyridazine-4-carbonyl]bicyclo[3.2.1]octane-2,4-dione; I+2-[2-(3,4-dimethoxyphenyl)-6-methyl-3-oxo-pyridazine-4-carbonyl]-5-methyl-cyclohexane-1,3-dione; I+2-[2-(3,4-dimethoxyphenyl)-6-methyl-3-oxo-pyridazine-4-carbonyl]cyclohexane-1,3-dione; I+2-[2-(3,4-dimethoxyphenyl)-6-methyl-3-oxo-pyridazine-4-carbonyl]-5,5-dimethyl-cyclohexane-1,3-dione; I+6-[2-(3,4-dimethoxyphenyl)-6-methyl-3-oxo-pyridazine-4-carbonyl]-2,2,4,4-tetramethyl-cyclohexane-1,3,5-trione; I+2-[2-(3,4-dimethoxyphenyl)-6-methyl-3-oxo-pyridazine-4-carbonyl]-5-ethyl-cyclohexane-1,3-dione; I+2-[2-(3,4-dimethoxyphenyl)-6-methyl-3-oxo-pyridazine-4-carbonyl]-4,4,6,6-tetramethyl-cyclohexane-1,3-dione; I+2-[6-cyclopropyl-2-(3,4-dimethoxyphenyl)-3-oxo-pyridazine-4-carbonyl]-5-methyl-cyclohexane-1,3-dione; I+3-[6-cyclopropyl-2-(3,4-dimethoxyphenyl)-3-oxo-pyridazine-4-carbonyl]bicyclo[3.2.1]octane-2,4-dione; I+2-[6-cyclopropyl-2-(3,4-dimethoxyphenyl)-3-oxo-pyridazine-4-carbonyl]-5,5-dimethyl-cyclohexane-1,3-dione; I+6-[6-cyclopropyl-2-(3,4-dimethoxyphenyl)-3-oxo-pyridazine-4-carbonyl]-2,2,4,4-tetramethyl-cyclohexane-1,3,5-trione; I+2-[6-cyclopropyl-2-(3,4-dimethoxyphenyl)-3-oxo-pyridazine-4-carbonyl]cyclohexane-1,3-dione; I+4-[2-(3,4-dimethoxyphenyl)-6-methyl-3-oxo-pyridazine-4-carbonyl]-2,2,6,6-tetramethyl-tetrahydropyran-3,5-dione and I+4-[6-cyclopropyl-2-(3,4-dimethoxyphenyl)-3-oxo-pyridazine-4-carbonyl]-2,2,6,6-tetramethyl-tetrahydropyran-3,5-dione.

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Preferred combinations (wherein “I” represents a compound of formula (I)) include: —I+benoxacor, I+cloquintocet (including cloquintocet-mexyl); I+cyprosulfamide; I+dichlormid; I+fenchlorazole (including fenchlorazole-ethyl); I+fenclorim; I+fluxofenim; 1+furilazole I+isoxadifen (including isoxadifen-ethyl); I+mefenpyr (including mefenpyr-diethyl); I+metcamifen;

I+N-(2-methoxybenzoyl)-4-[(methylaminocarbonyl)amino] benzenesulfonamide and I+oxabetrinil.

Particularly preferred are mixtures of a compound of formula (I) with cyprosulfamide, isoxadifen (including isoxadifen-ethyl), cloquintocet (including cloquintocet-mexyl) and/or N-(2-methoxybenzoyl)-4-[(methyl-aminocarbonyl)amino]benzenesulfonamide.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicummiliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

The compounds of formula (I) are also useful for pre-harvest desiccation in crops, for example, but not limited to, potatoes, soybean, sunflowers and cotton. Pre-harvest desiccation is used to desiccate crop foliage without significant damage to the crop itself to aid harvesting.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulphate | 3% | — | 5% |
| sodium diisobutylnaphthalenesulfonate | — | 10% | 6% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20 |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruded granules | |
|---|---|
| Active ingredients | 15% |
| sodium lignosulfonate | 2% |
| Carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

| Suspension concentrate | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| Carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

| Flowable concentrate for seed treatment | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a 20% solution | 0.5% |
| in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 Parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

### List of Abbreviations:

Boc=tert-butyloxycarbonyl
br=broad
CDCl₃=chloroform-d
CD₃OD=methanol-d
° C.=degrees Celsius
D₂O=water-d
DCM=dichloromethane
d=doublet
dd=double doublet
dt=double triplet
DMSO=dimethylsulfoxide
EtOAc=ethyl acetate
h=hour(s)
HCl=hydrochloric acid
HPLC=high-performance liquid chromatography (description of the apparatus and the
methods used for HPLC are given below)
m=multiplet
M=molar
min=minutes
MHz=mega hertz
mL=millilitre
mp=melting point
ppm=parts per million
q=quartet
quin=quintet
rt=room temperature
s=singlet
t=triplet
THE=tetrahydrofuran

### LC/MS=Liquid Chromatography Mass Spectrometry

### Preparative Reverse Phase HPLC Method:

Compounds purified by mass directed preparative HPLC using ES+/ES− on a Waters FractionLynx Autopurification system comprising a 2767 injector/collector with a 2545 gradient pump, two 515 isocratic pumps, SFO, 2998 photodiode array (Wavelength range (nm): 210 to 400), 2424 ELSD and QDa mass spectrometer. A Waters Atlantis T3 5 micron 19×10 mm guard column was used with a Waters Atlantis T3 OBD, 5 micron 30×100 mm prep column.
Ionisation method: Electrospray positive and negative: Cone (V) 20.00, Source Temperature (° C.)

### 120, Cone Gas Flow (L/Hr.) 50

Mass range (Da): positive 100 to 800, negative 115 to 800.

The preparative HPLC was conducted using an 11.4 minute run time (not using at column dilution, bypassed with the column selector), according to the following gradient table:

| Time (mins) | Solvent A (%) | Solvent B (%) | Flow (ml/min) |
|---|---|---|---|
| 0.00 | 100 | 0 | 35 |
| 2.00 | 100 | 0 | 35 |
| 2.01 | 100 | 0 | 35 |
| 7.0 | 90 | 10 | 35 |
| 7.3 | 0 | 100 | 35 |
| 9.2 | 0 | 100 | 35 |
| 9.8 | 99 | 1 | 35 |
| 11.35 | 99 | 1 | 35 |
| 11.40 | 99 | 1 | 35 |

515 pump 0 ml/min Acetonitrile (ACD)
515 pump 1 ml/min 90% Methanol/10% Water (make up pump)
Solvent A: Water with 0.05% Trifluoroacetic Acid
Solvent B: Acetonitrile with 0.05% Trifluoroacetic Acid

### PREPARATION EXAMPLES

### Example 1: Preparation of 2,3-dimethylcinnolin-2-ium-4-carboxylate A61

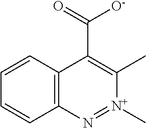

### Step 1: Preparation of 3-methylcinnolin-4-ol

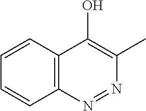

To a mixture of 1-(2-aminophenyl)propan-1-one (22 g) and glacial acetic acid (22 mL) was added 2M aqueous hydrochloric acid (66 mL) and water (22 mL). The mixture was cooled to 0° C. and a solution of sodium nitrite (11.192 g) in water (44 mL) was added slowly, keeping the temperature between 0° C. and 5° C. The mixture was stirred at 0° C. for one hour and urea (0.886 g) was added and stirred for another hour. To this was added a solution of sodium acetate (159.19 g) in water (440 mL) followed by dichloromethane (110 mL) at 0° C. and then the mixture was allowed to warm to room temperature and stirred for 15 hours. The reaction mass was filtered and the light brown solid was washed sequentially with water (50 mL), dichloromethane (20 mL) and hexane (20 mL) and dried to give 3-methylcinnolin-4-ol.

¹H NMR (400 MHz, CDCl₃) 12.50 (br. s., 1H) 8.15 (d, 1H) 7.48-7.60 (m, 1H) 7.39-7.47 (m, 1H) 7.19-7.31 (m, 1H) 2.34-2.35 (m, 3H) Step 2: Preparation of 4-chloro-3-methyl-cinnoline

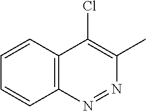

To a mixture of 3-methylcinnolin-4-ol (9 g) and chlorobenzene (90 mL), under a nitrogen atmosphere, was added 2-methylpyridine (1.0466 g) drop wise at room temperature. Phosphorus oxychloride (7.936 mL) was then added drop wise and the resulting mixture was heated at reflux for 2 hours. The reaction mass was poured cautiously into ice cold water and the resulting mixture was basified with saturated aqueous sodium carbonate solution. The reaction mass was extracted with dichloromethane (3×50 mL) and the combined organic layers were concentrated then purified by silica gel chromatography eluting with a 3:7 ration of ethyl acetate in iso-hexane to give 4-chloro-3-methyl-cinnoline.

¹H NMR (400 MHz, CDCl₃) 8.48 (m, 1H), 8.12 (m, 1H), 7.74-7.84 (m, 2H), 3.03 (s, 3H) Step 3: Preparation of 3-methyl-4-(p-tolylsulfonyl)cinnoline

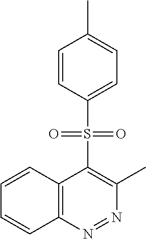 [SMILES: Cc1ccc(S(=O)(=O)c2c(C)nnc3ccccc23)cc1]

A mixture of 4-chloro-3-methyl-cinnoline (0.5 g) and acetonitrile (6 mL), under a nitrogen atmosphere, was cooled to 0° C. and sodium p-toluenesulfinate (0.549 g) was added in one portion. The mixture was stirred cold for 1 hour and then allowed to warm to room temperature and stirred overnight. The reaction mixture was partitioned between water and ethyl acetate (100 mL), then extracted further ethyl acetate (2×100 mL). The combined organic layers were dried over sodium sulphate and concentrated to give 3-methyl-4-(p-tolylsulfonyl)cinnoline.

¹H NMR (400 MHz, CDCl₃) 9.15 (d, 1H), 8.62 (d, 1H), 7.81-7.92 (m, 4H), 7.32 (d, 2H), 3.35 (s, 3H), 2.41 (s, 3H)

### Step 4: Preparation of 3-methylcinnoline-4-carbonitrile

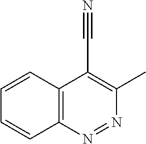

To a solution of 3-methyl-4-(p-tolylsulfonyl)cinnoline (2.5 g) in N,N-dimethylformamide (25 mL), under a nitrogen atmosphere, was added sodium cyanide (1.7 g) at room temperature. The reaction mixture was stirred for 2 hours then quenched with water and extracted with ethyl acetate (3×100 mL). The combined organic layers were dried over sodium sulphate and concentrated to give crude 3-methylcinnoline-4-carbonitrile which was used without further purification.

### Step 5: Preparation of 3-methylcinnoline-4-carboxylic Acid

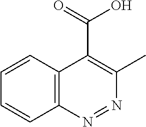

To a mixture of crude 3-methylcinnoline-4-carbonitrile (1 g) and water (8 mL) was added concentrated sulfuric acid (8 mL) drop wise. The reaction mixture was heated at 80° C. for 10 days. The reaction mixture was diluted with water (20 mL), basified with aqueous 2M sodium hydroxide, washed with ethyl acetate (3×100 mL) and the aqueous phase was acidified with 2M aqueous hydrochloric acid. The crude product was extracted with ethyl acetate (3×100 mL) and the combined organic layers were concentrated to give 3-methylcinnoline-4-carboxylic acid.

¹H NMR (400 MHz, CD₃OD) 8.49 (d, 1H) 8.09 (d, 1H) 7.95 (td, 2H) 2.99 (s, 3H) (CO₂H proton missing)

### Step 6: Preparation of 2,3-dimethylcinnolin-2-ium-4-carboxylate A61

To a solution of 3-methylcinnoline-4-carboxylic acid (300 mg) in tetrahydrofuran (9 mL) and 1,4-dioxane (9 mL) was added dimethyl sulphate (0.603 g) drop wise at room temperature under a nitrogen atmosphere. The reaction mixture was stirred at room temperature for 50 hours then concentrated and washed sequentially with tert-butyl methyl ether (2×20 mL) and acetone (10 mL). The resulting solid was purified by preparative reverse phase HPLC to give 2,3-dimethylcinnolin-2-ium-4-carboxylate.

¹H NMR (400 MHz, CD₃OD) 8.38-8.50 (m, 1H), 8.14-8.23 (m, 3H), 4.84 (s, 3H), 2.95-3.13 (m, 3H)

### Example 2: Preparation of ethyl-(2-methylcinnolin-2-ium-4-yl)phosphinate A48

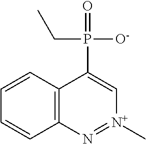 [SMILES: CCP(=O)([O-])c1c[n+](C)nc2ccccc12]

### Step 1: Preparation of 4-(p-tolylsulfonyl)cinnoline

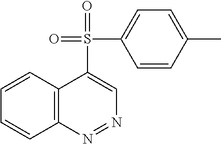 [SMILES: Cc1ccc(S(=O)(=O)c2cnnc3ccccc23)cc1]

To a solution of 4-chloro-cinnoline (24 g) in N,N-dimethylformamide (200 mL) was added with sodium p-toluenesulfonate (31.2 g) at room temperature. The reaction mixture was stirred at room temperature for 16 hours then quenched into ice water. The resulting solid was filtered and dried to afford 4-(p-tolylsulfonyl)cinnoline as a pale yellow solid.

¹H NMR (400 MHz, CDCl₃) 9.75 (s, 1H), 8.74-8.67 (m, 2H), 7.94-7.92 (d, 4H), 7.36-7.34 (d, 2H), 2.41 (s, 3H)

### Step 2: Preparation of 1-ethylphosphonoyloxyethane

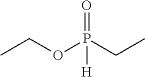

To a solution of triethyl phosphite (10 g) in tetrahydrofuran (100 mL) was added ethyl magnesium bromide (1.8 mL, 1 M in tetrahydrofuran) at room temperature. The reaction mixture was heated at 80° C. for 16 hours then quenched with 2M aqueous hydrochloric acid (75 mL). The crude product was extracted with ethyl acetate (3×100 mL), dried over sodium sulfate then concentrated. Purification by silica gel chromatography eluting with 0-80% ethyl acetate in iso-hexane afforded 1-ethylphosphonoyloxyethane as a pale yellow oil.

¹H NMR (400 MHz, CDCl₃) 7.72 (s, 5H), 6.40 (s, 0.5H), 4.20-4.09 (m, 2H), 1.83-1.77 (m, 2H), 1.39-1.35 (t, 3H), 1.19-1.12 (m, 3H)

### Step 3: Preparation of 4-[ethoxy(ethyl)phosphoryl]cinnoline

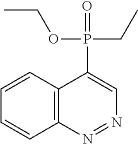 [SMILES: CCOP(=O)(CC)c1cnnc2ccccc12]

To a solution of 1-ethylphosphonoyloxyethane (1.28 g) in tetrahydrofuran (20 mL) at 78° C. was added lithium bis(trimethylsilyl)amide (1M in tetrahydrofuran, 10.5 mL) under a nitrogen atmosphere. The mixture was stirred at −78° C. for 1 hour then a solution of 4-(p-tolylsulfonyl)cinnoline (1.00 g) in tetrahydrofuran (10.0 mL) was added to the reaction mixture drop wise at this temperature. The resulting reaction mixture was allowed to warm to room temperature and stirred for 2 hours. The reaction mixture was quenched with saturated aqueous ammonium chloride (20.0 mL) and extracted with ethyl acetate (3×30 mL). The combined organic phase was dried over sodium sulfate, concentrated, then purified by silica gel chromatography eluting with 0-50% ethyl acetate in iso-hexane to give 4-[ethoxy(ethyl)phosphoryl]cinnoline as a yellow oil.

¹H NMR (300 MHz, CDCl₃) 9.59-9.56 (d, 1H), 8.70-8.66 (m, 2H), 7.98-7.87 (m, 2H), 4.30-3.99 (m, 2H), 2.18-1.96 (m, 2H), 1.41-1.36 (t, 3H), 1.19-1.07 (m, 3H)

### Step 4: Preparation of 4-[ethoxy(ethyl)phosphoryl]-2-methyl-cinnolin-2-ium iodide

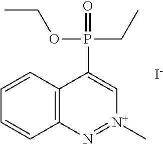 [SMILES: CCOP(=O)(CC)c1c[n+](C)nc2ccccc12 ; [I-]]

To a solution of 4-[ethoxy(ethyl)phosphoryl]cinnoline (0.65 g) in tetrahydrofuran (20 mL) was added iodomethane (0.49 mL) at room temperature. The reaction mixture was stirred at room temperature for 16 hours then concentrated and triturated with acetone to afford 4-[ethoxy(ethyl)phosphoryl]-2-methyl-cinnolin-2-ium iodide as a brown solid.

¹H NMR (300 MHz, DMSO-d₆) 9.94-9.91 (d, 1H), 8.94-8.91 (d, 1H), 8.78-8.75 (d, 1H), 8.54-8.42 (m, 2H), 4.95 (s, 3H), 4.24-3.97 (m, 2H), 2.39-2.16 (m, 2H), 1.31-1.27 (t, 3H), 1.10-1.04 (m, 3H)

### Step 5: Preparation of ethyl-(2-methylcinnolin-2-ium-4-yl)phosphinate A48

A mixture of 4-[ethoxy(ethyl)phosphoryl]-2-methyl-cinnolin-2-ium iodide (0.78 g) and concentrated aqueous hydrochloric acid (15 mL) was heated at 100° C. for 16 hours. After cooling to room temperature solvents were removed in vacuo and the residue was concentrated and triturated with acetone (10 mL) to afford ethyl-(2-methylcinnolin-2-ium-4-yl)phosphinate as a black gum.

¹H NMR (400 MHz, D₂O) 9.38-9.36 (d, 1H), 8.79-8.77 (d, 1H), 8.50-8.47 (d, 1H), 8.27-8.18 (m, 2H), 4.79 (s, 3H), 1.88-1.83 (m, 2H), 0.91-0.82 (m, 3H)

### Example 3: Preparation of 2-methylcinnolin-2-ium-4-carboxylic Acid Methyl Sulfate A3

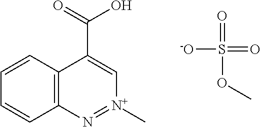

To a solution of cinnoline-4-carboxylic acid (0.5 g) in toluene (9 mL) was added dimethyl sulfate (0.532 g) drop wise at room temperature under a nitrogen atmosphere. The mixture was heated at 110° C. for 2 hours then cooled to room temperature and concentrated. To this crude product was added acetone followed by heating at reflux for 5 minutes with vigorous stirring. After cooling the resulting precipitate was filtered and dried to give 2-methylcinnolin-2-ium-4-carboxylic acid methyl sulfate as a dark blue/green solid.

¹H NMR (400 MHz, CD₃OD) 9.65 (d, 1H), 9.20 (d, 1H), 8.64-8.58 (m, 1H), 8.46-8.41 (m, 1H), 8.39-8.31 (m, 1H), 4.94 (s, 3H), 3.66 (s, 3H) (CO₂H proton missing)

### Example 4: Preparation of N-methoxy-2-methyl-cinnolin-2-ium-4-carboxamide 2,2,2-trifluoroacetate A4

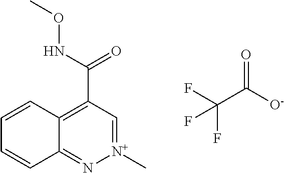 [SMILES: CONC(=O)c1c[n+](C)nc2ccccc12 ; O=C([O-])C(F)(F)F]

### Step 1: Preparation of N-methoxycinnoline-4-carboxamide

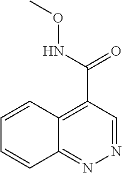

A mixture of cinnoline-4-carboxylic acid (0.5 g), O-(benzotriazol-1-yl)-N,N,N′,N′-tetramethyluronium tetrafluoroborate (1.03 g) and methoxyammonium chloride (0.264 g) was stirred in acetonitrile (14.4 mL) under a nitrogen atmosphere at room temperature. Triethylamine (0.734 g) was added and the reaction was stirred at room temperature for 3.5 hours. The reaction mixture was concentrated and the residue partitioned between 2M aqueous hydrochloric acid and dichloromethane. The aqueous layer was extracted with further dichloromethane and the combined organic phase was dried over magnesium sulfate and concentrated. The resulting solid was triturated with acetone, filtered then dried to give crude N-methoxycinnoline-4-carboxamide which was used without further purification.

### Step 2: Preparation of N-methoxy-2-methyl-cinnolin-2-ium-4-carboxamide 2,2,2-trifluoroacetate A4

Crude N-methoxycinnoline-4-carboxamide from Step 1 was stirred in iodomethane (5.70 g) at room temperature for 16 hours. The reaction mixture was concentrated and the residue partitioned between water and dichloromethane. The aqueous layer was concentrated then purified by preparative reverse phase HPLC (trifluoroacetic acid was present in the eluent) to give N-methoxy-2-methyl-cinnolin-2-ium-4-carboxamide 2,2,2-trifluoroacetate as a red/brown gum.

¹H NMR (400 MHz, CD₃OD) 9.94 (s, 1H), 8.94 (br. s., 1H), 8.65 (d, 1H), 8.49-8.27 (m, 2H), 4.94 (s, 3H), 4.05 (s, 3H) (NH proton missing)

### Example 5: Preparation of (2-methylcinnolin-2-ium-4-carbonyl)-methylsulfonyl-azanide

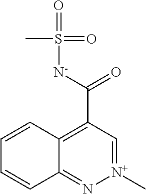 [SMILES: C[n+]1cc(C(=O)[N-]S(C)(=O)=O)c2ccccc2n1]

### Step 1: N-methylsulfonylcinnoline-4-carboxamide

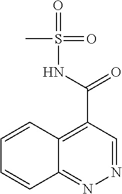 [SMILES: CS(=O)(=O)NC(=O)c1cnnc2ccccc12]

A mixture of cinnoline-4-carboxylic acid (0.3 g), N,N-dimethylaminopyridine (0.276 g), methanesulfonamide (0.217 g) and N-(3-dimethylaminopropyl)-N′-ethylcarbodiimide hydrochloride (0.438 g) was stirred in dichloromethane (12.1 mL) under a nitrogen atmosphere at room temperature for 19 hours. The reaction mixture was concentrated and purified by preparative reverse phase HPLC to afford N-methylsulfonylcinnoline-4-carboxamide as an orange gum.

¹H NMR (400 MHz, CDCl₃) 9.43 (br s, 1H), 8.59-8.54 (m, 1H), 8.51-8.45 (m, 1H), 8.00-7.91 (m, 2H), 3.53 (s, 3H) (NH proton missing)

### Step 2: Preparation of (2-methylcinnolin-2-ium-4-carbonyl)-methylsulfonyl-azanide A5

A mixture of N-methylsulfonylcinnoline-4-carboxamide (0.18 g) and iodomethane (3.42 g) was stirred at room temperature for 21 hours. The resulting solid was then filtered and dried to afford (2-methylcinnolin-2-ium-4-carbonyl)-methylsulfonyl-azanide as a brown solid.

1H NMR (400 MHz, CD₃OD) 9.87 (s, 1H), 9.24-9.11 (m, 1H), 8.63-8.55 (m, 1H), 8.39-8.26 (m, 2H), 4.84-4.77 (m, 3H), 3.24 (s, 3H)

### Example 6: Preparation of 2-methylcinnolin-2-ium-4-sulfonate A6

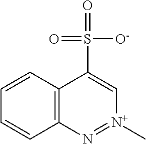

### Step 1: Preparation of cinnoline-4-sulfonic Acid

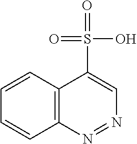 [SMILES: O=S(=O)(O)c1cnnc2ccccc12]

To a suspension of 4-chlorocinnoline (0.2 g) in water (4 mL) was added sodium sulfite (0.234 g) and the mixture was heated at 100° C. for 1 hour. The reaction mixture was concentrated to give cinnoline-4-sulfonic acid as a yellow solid.

¹H NMR (400 MHz, D₂O) 9.50 (s, 1H), 8.56-8.48 (m, 1H), 8.48-8.40 (m, 1H), 8.02-7.91 (m, 2H)

### Step 2: Preparation of 2-methylcinnolin-2-ium-4-sulfonate A6

To a mixture of cinnoline-4-sulfonic acid (0.11 g) in toluene (2.62 mL) was added dimethyl sulfate (0.08 g) and the mixture was heated at 110° C. for 2 hours under a nitrogen atmosphere. The reaction mixture was concentrated then purified by preparative reverse phase HPLC to afford 2-methylcinnolin-2-ium-4-sulfonate as a beige solid.

¹H NMR (400 MHz, D₂O) 9.74 (s, 1H), 8.77 (d, 1H), 8.57 (d, 1H), 8.39-8.33 (m, 1H), 8.32-8.25 (m, 1H), 4.85 (s, 3H)

### Example 7: Preparation of (2R)-2-[(2-methylcinnolin-2-ium-4-carbonyl)amino]propanoic acid 2,2,2-trifluoroacetate A9

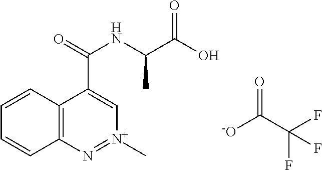

### Step 1: Preparation of tert-butyl (2R)-2-(cinnoline-4-carbonylamino)propanoate

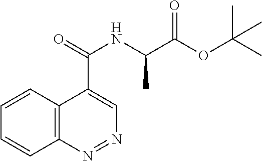 [SMILES: C[C@@H](NC(=O)c1cnnc2ccccc12)C(=O)OC(C)(C)C]

A mixture of cinnoline-4-carboxylic acid (0.5 g) and [(1S)-2-tert-butoxy-1-methyl-2-oxo-ethyl]ammonium chloride (0.574 g) in dichloromethane (14.4 mL) was cooled to 0° C. and pyridine (0.751 mL) was added drop wise, followed by the addition of dicyclohexylcarbodiimide (0.718 g) in one portion. The reaction mixture was allowed to warm to room temperature and stirred for 1 hour. The reaction mixture was filtered and the filtrate was concentrated and partitioned between water and ethyl acetate. The organic layer was washed sequentially with water, 0.1M aqueous hydrochloric acid and brine, then dried with magnesium sulfate and concentrated to give tert-butyl (2R)-2-(cinnoline-4-carbonylamino)propanoate as a dark red gum.

¹H NMR (400 MHz, CDCl₃) 9.38 (s, 1H), 8.61 (dd, 1H), 8.39 (dd, 1H), 7.95-7.82 (m, 2H), 6.86 (d, 1H), 4.83-4.70 (m, 1H), 1.59 (d, 3H), 1.53 (s, 9H)

### Step 2: Preparation of tert-butyl (2R)-2-[(2-methylcinnolin-2-ium-4-carbonyl)amino]propanoate Iodide A8

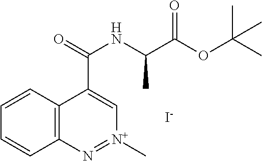 [SMILES: C[C@@H](NC(=O)c1c[n+](C)nc2ccccc12)C(=O)OC(C)(C)C ; [I-]]

A mixture of methyl iodide (1.33 mL) and tert-butyl (2R)-2-(cinnoline-4-carbonylamino)propanoate (0.2 g) were stirred at room temperature for 20 hours. The reaction mixture was concentrated and the residue was triturated with ethyl acetate to afford tert-butyl (2R)-2-[(2-methylcinnolin-2-ium-4-carbonyl)amino]propanoate iodide as an orange solid.

¹H NMR (400 MHz, D₂O) 9.75 (s, 1H), 8.70-8.64 (m, 1H), 8.60-8.52 (m, 1H), 8.47-8.38 (m, 2H), 4.96 (s, 3H), 4.65 (d, 1H), 1.60-1.50 (m, 12H) (NH proton missing)

### Step 3: Preparation of (2R)-2-[(2-methylcinnolin-2-ium-4-carbonyl)amino]propanoic acid 2,2,2-trifluoroacetate A9

A mixture of tert-butyl (2R)-2-[(2-methylcinnolin-2-ium-4-carbonyl)amino]propanoate iodide (0.14 g) and trifluoroacetic acid (0.947 mL) was stirred at room temperature for 2 hours. The reaction mixture was concentrated then recrystallised with ethyl acetate to afford (2R)-2-[(2-methylcinnolin-2-ium-4-carbonyl)amino]propanoic acid as an orange solid.

¹H NMR (400 MHz, D₂O) 9.77 (s, 1H), 8.71-8.64 (m, 1H), 8.60-8.50 (m, 1H), 8.48-8.33 (m, 2H), 4.95 (s, 3H), 1.61 (d, 3H) (one CH proton hidden underwater peak, NH and CO₂H protons missing)

### Example 8: Preparation of 2-methyl-N-(methylsulfamoyl)cinnolin-2-ium-4-carboxamide A10

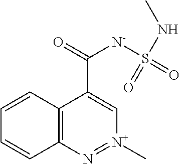 [SMILES: CNS(=O)(=O)[N-]C(=O)c1c[n+](C)nc2ccccc12]

### Step 1: Preparation of N-(methylsulfamoyl)cinnoline-4-carboxamide

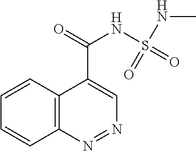 [SMILES: CNS(=O)(=O)NC(=O)c1cnnc2ccccc12]

A mixture of cinnoline-4-carboxylic acid (0.3 g) and 1,1′-carbonyldiimidazole (0.342 g) was heated in tetrahydrofuran (8.61 mL) at 70° C. for 1 hour under a nitrogen atmosphere. The mixture was cooled to room temperature and (sulfamoylamino)methane (0.228 g) and 1,8-diazabicyclo[5.4.0]undec-7-ene (0.342 mL) were added sequentially. The reaction mixture was stirred at room temperature for 22 hours, concentrated, then purified by preparative reverse phase HPLC to afford N-(methylsulfamoyl)cinnoline-4-carboxamide as a pale yellow gum.

¹H NMR (400 MHz, CDCl₃) 9.39 (s, 1H), 8.62 (d, 1H), 8.42 (d, 1H), 8.05-7.91 (m, 2H), 2.86 (s, 3H) (two NH protons missing)

### Step 2: Preparation of 2-methyl-N-(methylsulfamoyl)cinnolin-2-ium-4-carboxamide A10

A mixture of methyl iodide (1.1 mL) and N-(methylsulfamoyl)cinnoline-4-carboxamide (0.06 g) were stirred at room temperature for 4 hours. The resulting solid was filtered then washed with acetone to afford 2-methyl-N-(methylsulfamoyl)cinnolin-2-ium-4-carboxamide iodide as a pale orange solid.

¹H NMR (400 MHz, CD₃OD) 10.04 (s, 1H), 8.69 (dd, 2H), 8.56-8.37 (m, 2H), 4.97 (s, 3H), 2.85 (s, 3H) (NH proton missing)

### Example 9: Preparation of [2-(2,2-difluoroethyl)cinnolin-2-ium-4-carbonyl]-methylsulfonyl-azanide A14

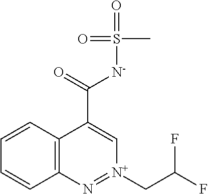 [SMILES: CS(=O)(=O)[N-]C(=O)c1c[n+](CC(F)F)nc2ccccc12]

A mixture of 2,2-difluoroethyl trifluoromethanesulfonate (0.522 g) and N-methylsulfonylcinnoline-4-carboxamide (200 mg) in acetonitrile (5 mL) was heated at 80° C. overnight. The reaction mixture was cooled and the resulting solid was filtered and dried to give [2-(2,2-difluoroethyl)cinnolin-2-ium-4-carbonyl]-methylsulfonyl-azanide.

¹H NMR (400 MHz, CD₃OD) 10.16 (s, 1H), 8.80-8.73 (m, 2H), 8.58-8.47 (m, 2H), 6.87-6.56 (m, 1H), 5.68 (dt, 2H), 3.52 (s, 3H)

### Example 10: Preparation of 2-methyl-N-(2-methyl-1,2,4-triazol-3-yl)cinnolin-2-ium-4-carboxamide Iodide A25

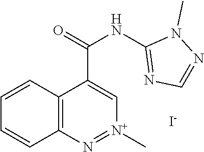 [SMILES: Cn1ncnc1NC(=O)c1c[n+](C)nc2ccccc12 ; [I-]]

### Step 1: Preparation of N-(2-methyl-1,2,4-triazol-3-yl)cinnoline-4-carboxamide

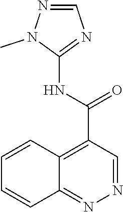 [SMILES: Cn1ncnc1NC(=O)c1cnnc2ccccc12]

A mixture of cinnoline-4-carboxylic acid (0.3 g), triethylamine (0.485 mL) and 1-methyl-1h-1,2,4-triazol-5-amine (0.203 g) was stirred in ethyl acetate (8.61 mL) at room temperature for 15 minutes. Propylphoshonic anhydride (2.05 mL) was added drop wise and the resulting mixture was stirred at room temperature for 20 hours. To this was added 0.5M aqueous hydrochloric acid (30 mL) followed by additional stirring for 2 hours. The resulting precipitate was filtered, washed with 0.5M aqueous hydrochloric acid then dried to afford N-(2-methyl-1,2,4-triazol-3-yl)cinnoline-4-carboxamide as a colourless solid.

¹H NMR (400 MHz, DMSO-d₆) 11.71 (br. s., 1H), 9.72 (br. s., 1H), 8.63 (d, 1H), 8.35 (br. s., 1H), 8.16-7.86 (m, 2H), 3.85 (br. s., 3H) (NH proton missing)

### Step 2: Preparation of 2-methyl-N-(2-methyl-1,2,4-triazol-3-yl)cinnolin-2-ium-4-carboxamide Iodide A25

A mixture of methyl iodide (0.123 mL), N-(2-methyl-1,2,4-triazol-3-yl)cinnoline-4-carboxamide (0.1 g) and methanol (1.18 mL) was heated at 60° C. for 24 hours. The resulting precipitate was filtered, washed with acetone then dried to afford 2-methyl-N-(2-methyl-1,2,4-triazol-3-yl)cinnolin-2-ium-4-carboxamide iodide as an orange solid.

¹H NMR (400 MHz, D₂O) 9.78 (s, 1H), 8.89-8.78 (m, 1H), 8.57-8.49 (m, 1H), 8.31-8.22 (m, 2H), 8.20 (s, 1H), 4.85 (s, 3H), 3.76 (s, 3H) (NH proton missing)

### Example 11: Preparation of ethoxy-[(2-methylcinnolin-2-ium-4-yl)methyl]phosphinic Acid A51

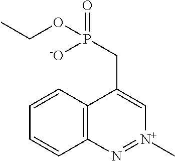

### Step 1: Preparation of ethyl 2-cinnolin-4-yl-2-diethoxyphosphoryl-acetate

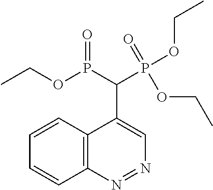

To a suspension of 4-(p-tolylsulfonyl)cinnoline (1 g) and caesium carbonate (5.74 g) in N,N-dimethylformamide (35.2 mL) was added ethyl 2-diethoxyphosphorylacetate (0.863 mL) and the reaction mixture was stirred at room temperature for 72 hours. The reaction mixture was partitioned between water (50 mL) and dichloromethane (200 mL). The organic phase was washed with water (5×50 mL), dried with sodium sulfate, concentrated then purified by silica gel chromatography eluting with 0 to 100% ethyl acetate in iso-hexane to give ethyl 2-cinnolin-4-yl-2-diethoxyphosphoryl-acetate as an orange oil.

¹H NMR (400 MHz, CD₃OD) 9.60 (d, 1H), 8.49-8.53 (m, 1H), 8.36-8.39 (m, 1H), 7.93-8.03 (m, 2H), 3.93-4.37 (m, 7H), 1.21-1.30 (m, 6H), 1.09 (t, 3H)

### Step 2: Preparation of cinnolin-4-ylmethyl(ethoxy)phosphinic Acid

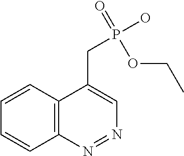 [SMILES: CCOP(=O)(O)Cc1cnnc2ccccc12]

A mixture of ethyl 2-cinnolin-4-yl-2-diethoxyphosphoryl-acetate (300 mg) and 2.5M aqueous sodium hydroxide (2 mL) was heated at reflux for 2 hours. The reaction mixture was neutralised with saturated aqueous ammonium chloride and washed with dichloromethane. The aqueous layer was concentrated, stirred in acetone, filtered then dried to give cinnolin-4-ylmethyl(ethoxy)phosphinic acid as a green oil.

¹H NMR (400 MHz, CD₃OD) 9.27 (d, 1H), 8.42-8.33 (m, 2H), 8.00-7.88 (m, 2H), 3.91-3.81 (m, 2H), 3.62-3.51 (m, 2H), 1.14 (t, 3H) (POH proton missing)

### Step 3: Preparation of ethoxy-[(2-methylcinnolin-2-ium-4-yl)methyl]phosphinic Acid A51

To a mixture of cinnolin-4-ylmethyl(ethoxy)phosphinic acid (220 mg), acetone (2 mL) and iodomethane (0.543 mL) was added a minimum amount of methanol. The solution was stirred at room temperature overnight, concentrated, then purified by preparative reverse phase HPLC to afford ethoxy-[(2-methylcinnolin-2-ium-4-yl)methyl]phosphinic acid.

¹H NMR (400 MHz, CD₃OD) 9.57 (d, 1H), 8.60-8.68 (m, 1H), 8.48-8.57 (m, 1H), 8.27-8.35 (m, 2H), 4.89 (s, 3H), 4.00 (quin, 2H), 3.82-3.93 (m, 2H), 1.21 (t, 3H) (Some exchange of CH₂ protons)

### Example 12: Preparation of (2-methylcinnolin-2-ium-4-yl)methylphosphonic Acid A50

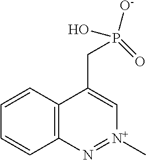 [SMILES: C[n+]1cc(CP(=O)([O-])O)c2ccccc2n1]

### Step 1: Preparation of 4-(diethoxyphosphorylmethyl)cinnoline

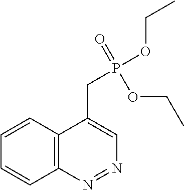 [SMILES: CCOP(=O)(Cc1cnnc2ccccc12)OCC]

A mixture of ethyl 2-cinnolin-4-yl-2-diethoxyphosphoryl-acetate (250 mg) and 0.5M aqueous sodium hydroxide (1 mL) was heated at 60° C. for 90 minutes. The reaction mixture was neutralised with saturated aqueous ammonium chloride and washed with dichloromethane. The organic layer was concentrated then purified by silica gel chromatography eluting with 0 to 10% methanol in dichloromethane to give 4-(diethoxyphosphorylmethyl)cinnoline.

¹H NMR (400 MHz, CD₃OD) 9.27 (d, 1H), 8.39-8.48 (m, 1H), 8.31 (d, 1H), 7.85-8.01 (m, 2H), 4.09 (q, 4H), 3.90 (d, 2H), 1.22 (q, 6H)

### Step 2: Preparation of 4-(diethoxyphosphorylmethyl)-2-methyl-cinnolin-2-ium iodide A47

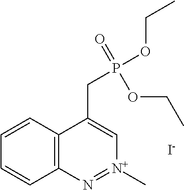 [SMILES: CCOP(=O)(Cc1c[n+](C)nc2ccccc12)OCC ; [I-]]

A mixture of 4-(diethoxyphosphorylmethyl)cinnoline (125 mg), acetone (2 mL) and iodomethane (0.139 mL) was stirred at room temperature overnight. The resulting precipitate was then filtered to afford 4-(diethoxyphosphorylmethyl)-2-methyl-cinnolin-2-ium iodide as a brown solid.

¹H NMR (400 MHz, CD₃OD) 9.69 (d, 1H), 8.59-8.68 (m, 2H), 8.33-8.42 (m, 2H), 4.93 (s, 3H), 4.07-4.25 (m, 4H), 1.27 (t, 6H). (Note: benzylic protons exchanged in the deuterated solvent)

### Step 3: Preparation of (2-methylcinnolin-2-ium-4-yl)methylphosphonic Acid A50

A mixture of 4-(diethoxyphosphorylmethyl)-2-methyl-cinnolin-2-ium (100 mg) and concentrated hydrochloric acid (1 mL) was heated at reflux for 3 hours. The reaction mixture was concentrated and purified by preparative reverse phase HPLC to afford (2-methylcinnolin-2-ium-4-yl)methylphosphonic acid.

¹H NMR (400 MHz, CD₃OD) 9.59 (d, 1H), 8.59-8.67 (m, 1H), 8.48-8.57 (m, 1H), 8.25-8.34 (m, 2H), 4.88 (s, 3H), 3.91 (d, 1H) (partial exchange of CH₂ at 3.91, POH proton missing)

### Example 13: Preparation of ethoxy-[2-(2-methylcinnolin-2-ium-4-yl)ethyl]phosphinic Acid 2,2,2-trifluoroacetate A54

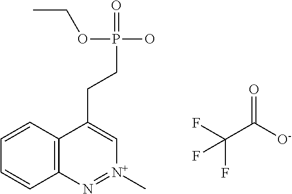 [SMILES: CCOP(=O)(O)CCc1c[n+](C)nc2ccccc12 ; O=C([O-])C(F)(F)F]

### Step 1: Preparation of 4-(2-diethoxyphosphorylethyl)cinnoline

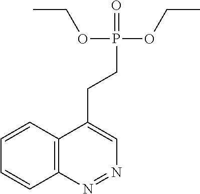 [SMILES: CCOP(=O)(CCc1cnnc2ccccc12)OCC]

A microwave vial was charged with 4-chlorocinnoline (0.5 g), 1-[ethoxy(vinyl)phosphoryl]oxyethane (0.934 mL), palladium (II) acetate (0.0341 g), tris-o-tolylphosphane (0.102 g), triethylamine (1.27 mL) and N,N-dimethylformamide (9.87 mL), purged with nitrogen then heated at 150° C. under microwave irradiation for 30 minutes. The reaction mixture was diluted with dichloromethane, concentrated, then purified by silica gel chromatography eluting with 0 to 10% methanol in dichloromethane to give 4-(2-diethoxyphosphorylethyl)cinnoline as an orange gum.

¹H NMR (400 MHz, CDCl₃) 9.21 (s, 1H), 8.58-8.51 (m, 1H), 8.10-8.03 (m, 1H), 7.91-7.78 (m, 2H), 4.22-4.07 (m, 4H), 3.45-3.34 (m, 2H), 2.27-2.14 (m, 2H), 1.36-1.31 (m, 6H)

### Step 2: Preparation of 4-(2-diethoxyphosphorylethyl)-2-methyl-cinnolin-2-ium iodide

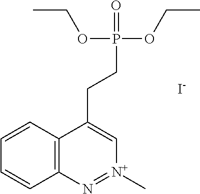 [SMILES: CCOP(=O)(CCc1c[n+](C)nc2ccccc12)OCC ; [I-]]

To a solution of 4-(2-diethoxyphosphorylethyl)cinnoline (0.129 g) in acetone (2.19 mL) was added iodomethane (0.273 mL) and lithium chloride (0.002 g). The reaction mixture was heated at 40° C. for 6 hours then left to stand overnight. The reaction mixture was concentrated to give 4-(2-diethoxyphosphorylethyl)-2-methyl-cinnolin-2-ium iodide as a brown gum, which was used without further purification.

¹H NMR (400 MHz, CD₃OD) 9.88 (s, 1H), 8.61-8.53 (m, 2H), 8.44-8.32 (m, 2H), 4.96 (s, 3H), 4.28-4.12 (m, 4H), 3.77-3.68 (m, 2H), 2.65-2.53 (m, 2H), 1.37-1.31 (m, 6H)

### Step 3: Preparation of ethoxy-[2-(2-methylcinnolin-2-ium-4-yl)ethyl]phosphinic acid 2,2,2-trifluoroacetate A54

A mixture of 4-(2-diethoxyphosphorylethyl)-2-methyl-cinnolin-2-ium iodide (0.19 g) and concentrated hydrochloric acid (1.74 mL) was heated at reflux for 3 hours. The reaction mixture was concentrated and purified by preparative reverse phase HPLC (trifluoroacetic acid was present in the eluent) to afford ethoxy-[2-(2-methylcinnolin-2-ium-4-yl)ethyl]phosphinic acid 2,2,2-trifluoroacetate as a yellow gum.

¹H NMR (400 MHz, CD₃OD) 9.67 (s, 1H), 8.62-8.54 (m, 2H), 8.39-8.30 (m, 2H), 4.90 (s, 3H), 4.11-4.02 (m, 2H), 3.67 (ddd, 2H), 2.36-2.25 (m, 2H), 1.28 (t, 3H) (POH proton missing)

### Example 14: Preparation of 2-(2-methylcinnolin-2-ium-4-yl)sulfanylacetic Acid Chloride

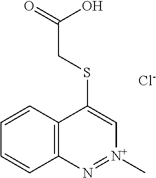 [SMILES: C[n+]1cc(SCC(=O)O)c2ccccc2n1 ; [Cl-]]

### Step 1: Preparation of methyl 2-cinnolin-4-ylsulfanylacetate

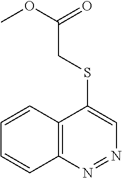 [SMILES: COC(=O)CSc1cnnc2ccccc12]

A mixture of methyl 2-sulfanylacetate (0.14 g), potassium carbonate (0.267 g), 4-(p-tolylsulfonyl)cinnoline (250 mg) and acetone (8.8 mL) was heated at reflux for 5 hours. The reaction mixture was filtered and concentrated to give methyl 2-cinnolin-4-ylsulfanylacetate as a yellow solid, which was used without further purification.

### Step 2: Preparation of methyl 2-(2-methylcinnolin-2-ium-4-yl)sulfanylacetate Iodide

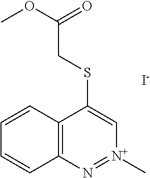 [SMILES: COC(=O)CSc1c[n+](C)nc2ccccc12 ; [I-]]

To a mixture of methyl 2-cinnolin-4-ylsulfanylacetate (194 mg), acetone (8.28 mL) and iodomethane (0.515 mL) was added a minimum amount of methanol. The solution was stirred at room temperature overnight, concentrated, then purified by preparative reverse phase HPLC to afford methyl 2-(2-methylcinnolin-2-ium-4-yl)sulfanylacetate iodide as an off white solid.

¹H NMR (400 MHz, CD₃OD) 9.59 (s, 1H), 8.50-8.55 (m, 1H), 8.43-8.48 (m, 1H), 8.26-8.34 (m, 2H), 4.85 (s, 3H), 4.50 (s, 2H), 3.83 (s, 3H)

### Step 3: Preparation of 2-(2-methylcinnolin-2-ium-4-yl)sulfanylacetic Acid Chloride A57

A mixture of methyl 2-(2-methylcinnolin-2-ium-4-yl)sulfanylacetate iodide (0.1 g) and concentrated hydrochloric acid (2 mL) was heated at 70° C. for 2 hours. The reaction mixture was concentrated to afford 2-(2-methylcinnolin-2-ium-4-yl)sulfanylacetic acid chloride.

¹H NMR (400 MHz, CD₃OD) 9.62 (s, 1H), 8.53-8.48 (m, 1H), 8.48-8.42 (m, 1H), 8.34-8.25 (m, 2H), 4.87 (s, 3H), 4.49 (s, 2H) (CO₂H proton missing)

### Example 15: Preparation of [2-[2-(methanesulfonamido)-2-oxo-ethyl]cinnolin-2-ium-4-yl]-methoxy-phosphinate A63

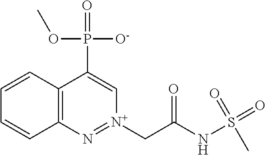 [SMILES: COP(=O)([O-])c1c[n+](CC(=O)NS(C)(=O)=O)nc2ccccc12]

### Step 1: Preparation of 2-bromo-N-methylsulfonyl-acetamide

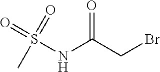

To a solution of methanesulfonamide (1 g) in toluene (63 mL) was added 2-bromoacetyl bromide (3.7 mL) drop wise at room temperature. The reaction was heated at 70° C. for 5 hours then cooled to room temperature. After further cooling over ice and the resulting precipitate was filtered, washed with cold toluene then dried to give 2-bromo-N-methylsulfonyl-acetamide as a pale yellow solid.

¹H NMR (400 MHz, CDCl₃) 8.81 (br s, 1H), 3.95 (s, 2H), 3.35 (s, 3H)

### Step 2: Preparation of [2-[2-(methanesulfonamido)-2-oxo-ethyl]cinnolin-2-ium-4-yl]-methoxy-phosphinate A63

To a mixture of 4-dimethoxyphosphorylcinnoline (200 mg) in acetone (2 mL) was added 2-bromo-N-methylsulfonyl-acetamide (362 mg) over 10 minutes. The mixture was stirred at room temperature for 2 days. The reaction mixture was partitioned between water and dichloromethane.

The aqueous layer was concentrated and purified by preparative reverse phase HPLC to give [2-[2-(methanesulfonamido)-2-oxo-ethyl]cinnolin-2-ium-4-yl]-methoxy-phosphinate as a brown foamy solid.

¹H NMR (400 MHz, D₂O) 9.46-9.56 (m, 1H) 8.75 (d, 1H) 8.55 (d, 1H) 8.22-8.41 (m, 2H) 5.91-5.99 (m, 2H) 3.51 (s, 3H) 3.17 (s, 3H) (NH proton missing)

### Example 16: Preparation of (2-tert-butylcinnolin-2-ium-4-yl)-ethoxy-phosphinate A75

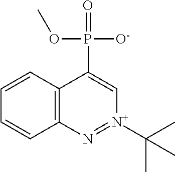 [SMILES: COP(=O)([O-])c1c[n+](C(C)(C)C)nc2ccccc12]

### Step 1: Preparation of 4-diethoxyphosphorylcinnoline

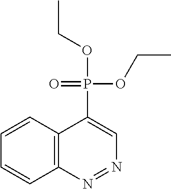 [SMILES: CCOP(=O)(OCC)c1cnnc2ccccc12]

To a stirred suspension of sodium hydride (0.106 g, 60% in mineral oil) in tetrahydrofuran (17.6 mL) was added diethyl phosphite (0.364 g) at 0° C. under a nitrogen atmosphere, followed by stirring for 30 minutes. This mixture was then added dropwise to an ice cold solution of 4-(p-tolylsulfonyl)cinnoline (0.5 g) in tetrahydrofuran (4.8 mL). After warming to room temperature the combined mixture was stirred for a further 2 hours then left to stand overnight. After dilution with water (50 mL) and extraction with dichloromethane (3×) the organic phase was washed sequentially with water and brine, then dried over magnesium sulfate and concentrated to give 4-diethoxyphosphorylcinnoline as a yellow gum.

¹H NMR (400 MHz, CDCl₃) 9.64 (d, 1H), 8.69-8.62 (m, 1H), 8.55-8.49 (m, 1H), 7.98-7.86 (m, 2H), 4.37-4.16 (m, 4H), 1.37 (t, 6H)

### Step 2: Preparation of 2-tert-butyl-4-diethoxyphosphoryl-cinnolin-2-ium perchlorate A73

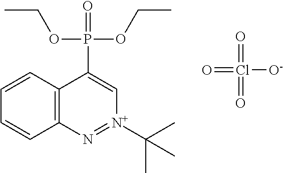 [SMILES: CCOP(=O)(OCC)c1c[n+](C(C)(C)C)nc2ccccc12 ; [O-][Cl+3]([O-])([O-])[O-]]

To a stirred solution of 4-diethoxyphosphorylcinnoline (0.6 g) in tert-butylacetate (10 mL) was added perchloric acid (1.06 mL) at room temperature. The reaction mixture was stirred at room temperature for 16 hours then quenched with ice, diluted with water (100 mL) and extracted with ethyl acetate (2×75 mL). The combined organic phase was dried over sodium sulfate then concentrated to give 2-tert-butyl-4-diethoxyphosphoryl-cinnolin-2-ium perchlorate as a brown liquid.

¹H NMR (400 MHz, D₂O) 9.65-9.63 (d, 1H), 8.69-8.67 (d, 1H), 8.61-8.59 (d, 1H), 8.40-8.29 (m, 2H), 4.34-4.18 (m, 4H), 1.90 (s, 9H), 1.28-1.26 (t, 6H)

### Step 3: Preparation of (2-tert-butylcinnolin-2-ium-4-yl)-ethoxy-phosphinate A73

A solution of 2-tert-butyl-4-diethoxyphosphoryl-cinnolin-2-ium perchlorate (0.3 g) in concentrated hydrochloric acid (10 mL) was stirred at room temperature for 72 hours. The reaction mixture was concentrated then purified by preparative reverse phase HPLC to give (2-tert-butylcinnolin-2-ium-4-yl)-ethoxy-phosphinate as a brown liquid.

¹H NMR (400 MHz, D₂O) 9.57-9.55 (d, 1H), 8.69-8.67 (d, 1H), 8.58-8.56 (d, 1H), 8.29-8.20 (m, 2H), 3.85-3.78 (m, 2H), 1.87 (s, 9H), 1.11-1.07 (t, 3H)

### Example 17: Preparation of isopropoxy-(2-isopropylcinnolin-2-ium-4-yl)phosphinate A74

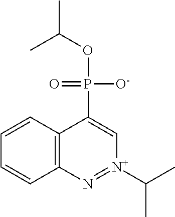 [SMILES: CC(C)OP(=O)([O-])c1c[n+](C(C)C)nc2ccccc12]

A mixture of 4-di-isopropylphosphorylcinnoline (0.4 g) and 2-iodopropane (6.46 mL) was heated for 1 hour at 100° C. under microwave irradiation. The reaction mixture was then filtered through diatomaceous earth, concentrated and purified by preparative reverse phase HPLC to give isopropoxy-(2-isopropylcinnolin-2-ium-4-yl)phosphinate as a light brown solid.

¹H NMR (400 MHz, D₂O) 9.46-9.43 (d, 1H), 8.70-8.68 (d, 1H), 8.54-8.52 (d, 1H), 8.28-8.19 (m, 2H), 5.46-5.39 (m, 1H), 4.45-4.37 (m, 1H), 1.72-1.70 (d, 6H), 1.08-1.07 (d, 6H)

### Example 18: Preparation of [2-(2-hydroxyethyl)cinnolin-2-ium-4-yl]-methoxy-phosphinate A68

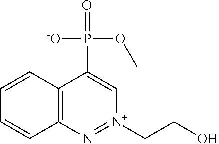

### Step 1: Preparation of cinnolin-4-yl(methoxy)phosphinic Acid

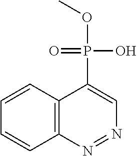

To mixture of 4-dimethoxyphosphorylcinnoline (3.41 g) and 1,4-dioxane (100 mL) was added aqueous 3M sodium hydroxide (24 mL) drop wise and the resulting mixture was stirred for 2 hours at room temperature. The reaction mixture was concentrated then partitioned between water and dichloromethane. The aqueous layer was acidified to pH 3 with concentrated hydrochloric acid, concentrated and the residue was stirred in methanol. After filtration the filtrate was concentrated then purified by preparative reverse phase HPLC to give cinnolin-4-yl(methoxy)phosphinic acid.

¹H NMR (400 MHz, CD₃OD) 9.58 (d, 1H), 8.87-8.82 (m, 1H), 8.64-8.59 (m, 1H), 8.23-8.17 (m, 2H), 3.67 (d, 3H) (POH proton missing)

### Step 2: Preparation of [2-(2-hydroxyethyl)cinnolin-2-ium-4-yl]-methoxy-phosphinate A68

A mixture of 1,3,2-dioxathiolane 2,2-dioxide (161 mg), cinnolin-4-yl(methoxy)phosphinic acid (290 mg) and 1,2-dichloroethane (5 mL) was heated at 85° C. overnight. The reaction mixture was concentrated and partitioned between water and dichloromethane. The aqueous layer was concentrated and purified by preparative reverse phase HPLC to give [2-(2-hydroxyethyl)cinnolin-2-ium-4-yl]-methoxy-phosphinate as a brown gum.

¹H NMR (400 MHz, D₂O) 9.45-9.51 (m, 1H) 8.71 (d, 1H) 8.51-8.57 (m, 1H) 8.20-8.33 (m, 2H) 5.15 (dd, 2H) 4.17-4.25 (m, 2H) 3.47-3.57 (m, 3H) (OH proton missing) Also isolated from this reaction was hydroxy-[2-(2-hydroxyethyl)cinnolin-2-ium-4-yl]phosphinate A67 as a brown gum.

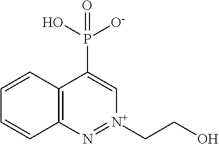 [SMILES: O=P([O-])(O)c1c[n+](CCO)nc2ccccc12]

¹H NMR (400 MHz, D₂O) 9.45-9.51 (m, 1H) 8.68-8.74 (m, 1H) 8.47-8.51 (m, 1H) 8.46 (s, 1H) 8.15-8.29 (m, 2H) 5.12 (dd, 2H) 4.14-4.26 (m, 2H) (OH or POH proton missing).

### Example 19: Preparation of hydroxy-(2-methylcinnolin-2-ium-4-yl)phosphinate A78

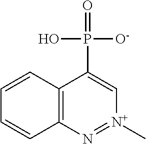

To a suspension of methoxy-(2-methylcinnolin-2-ium-4-yl)phosphinate (0.2 g) in dichloromethane (2 mL) was added bromotrimethylsilane (0.394 g) at room temperature. The reaction mixture was stirred for 5 hours then concentrated, triturated with acetone and dried to give hydroxy-(2-methylcinnolin-2-ium-4-yl)phosphinate as a pale brown solid.

¹H NMR (400 MHz, D₂O) 9.46 (d, 1H), 8.72 (d, 1H), 8.49 (d, 1H), 8.31-8.17 (m, 2H), 4.81 (s, 3H) (POH proton missing)

### Example 20: Preparation of [2-(2-methoxy-2-oxo-ethyl)cinnolin-2-ium-4-carbonyl]-methylsulfonyl-azanide A15

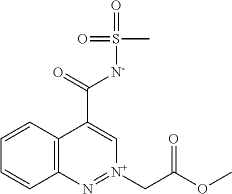 [SMILES: COC(=O)C[n+]1cc(C(=O)[N-]S(C)(=O)=O)c2ccccc2n1]

A mixture of methyl 2-bromoacetate (0.23 mL) and N-methylsulfonylcinnoline-4-carboxamide (0.2 g) in acetonitrile (5 mL) was heated at 80° C. overnight. The reaction mixture was concentrated and the residue partitioned between dichloromethane and water. The aqueous phase was concentrated and purified by preparative reverse phase HPLC to give [2-(2-methoxy-2-oxo-ethyl)cinnolin-2-ium-4-carbonyl]-methylsulfonyl-azanide, ¹H NMR (400 MHz, CD₃OD) 10.01 (s, 1H), 9.27-9.21 (m, 1H), 8.62-8.57 (m, 1H), 8.42-8.31 (m, 2H), 6.11-6.06 (m, 1H), 3.87 (s, 3H), 3.22 (s, 3H) (One proton at 6.11-6.06 exchanged out)

### Example 21: Preparation of cyclopropylsulfonyl-[2-(3-methoxy-3-oxo-propyl)cinnolin-2-ium-4-carbonyl]azanide A19

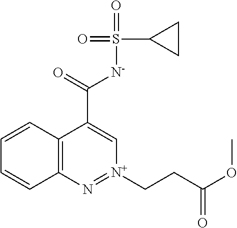 [SMILES: COC(=O)CC[n+]1cc(C(=O)[N-]S(=O)(=O)C2CC2)c2ccccc2n1]

A mixture of methyl 3-bromopropanoate (0.18 mL) and N-cyclopropylsulfonylcinnoline-4-carboxamide (0.15 g) in acetonitrile (4 mL) was heated at 80° C. overnight. A further aliquot of methyl 3-bromopropanoate (0.18 mL) was added and heating continued again overnight. The reaction mixture was concentrated and the residue partitioned between dichloromethane and water. The aqueous phase was concentrated and purified by preparative reverse phase HPLC to give cyclopropylsulfonyl-[2-(3-methoxy-3-oxo-propyl)cinnolin-2-ium-4-carbonyl]azanide.

¹H NMR (400 MHz, CD₃OD) 10.04 (s, 1H), 9.01-8.95 (m, 1H), 8.64-8.57 (m, 1H), 8.40-8.32 (m, 2H), 5.41 (t, 2H), 3.69 (s, 3H), 3.40 (t, 2H), 3.15-3.08 (m, 1H), 1.32-1.19 (m, 2H), 1.16-1.04 (m, 2H) [2-(2-carboxyethyl)cinnolin-2-ium-4-carbonyl]-cyclopropylsulfonyl-azanide A20, was also isolated from this reaction mixture

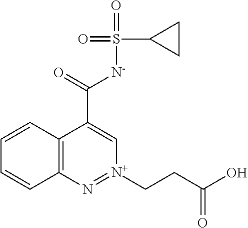 [SMILES: O=C(O)CC[n+]1cc(C(=O)[N-]S(=O)(=O)C2CC2)c2ccccc2n1]

¹H NMR (400 MHz, CD₃OD) 10.07 (s, 1H), 8.90-8.84 (m, 1H), 8.67-8.58 (m, 1H), 8.41-8.32 (m, 2H), 5.40 (t, 2H), 3.38 (t, 2H), 3.12 (tt, 1H), 1.30-1.23 (m, 2H), 1.16-1.08 (m, 2H) (CO₂H proton missing)

### Example 22: Preparation of 2-(carboxymethyl)cinnolin-2-ium-4-carboxylate A23

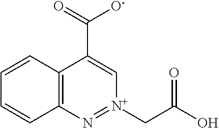 [SMILES: O=C(O)C[n+]1cc(C(=O)[O-])c2ccccc2n1]

A mixture of [2-(2-methoxy-2-oxo-ethyl)cinnolin-2-ium-4-carbonyl]-methylsulfonyl-azanide (0.25 g) and aqueous 2M hydrochloric acid (4 mL) was heated at 80° C. for 2 hours. The mixture was concentrated and triturated with acetone to give 2-(carboxymethyl)cinnolin-2-ium-4-carboxylate as a brown solid.

¹H NMR (400 MHz, D₂O) 9.68 (s, 1H), 8.77-8.73 (m, 1H), 8.53-8.48 (m, 1H), 8.32-8.21 (m, 2H), 5.85 (s, 2H) (CO₂H proton missing)

### Example 23: Preparation of 2-(4-carboxycinnolin-2-ium-2-yl)ethyl sulfate A28

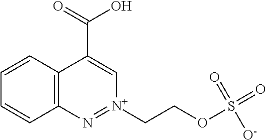 [SMILES: O=C(O)c1c[n+](CCOS(=O)(=O)[O-])nc2ccccc12]

To a mixture of cinnoline-4-carboxylic acid (0.4 g) and 1,2-dichloroethane (8 mL) was added 1,3,2-dioxathiolane 2,2-dioxide (0.312 g) and the mixture was heated at 85° C. overnight. The resulting precipitate was filtered off, washed with acetone and The reaction mixture was cooled to room temperature and allowed to stand overnight. The reaction mixture was concentrated 2-(4-carboxycinnolin-2-ium-2-yl)ethyl sulfate as a yellow solid.

¹H NMR (400 MHz, D₂O) 9.67 (s, 1H) 8.64-8.76 (m, 1H) 8.47-8.58 (m, 1H) 8.15-8.33 (m, 2H) 5.29-5.37 (m, 2H) 4.62-4.76 (m, 2H) (CH₂ underwater peak, CO₂H proton missing)

### Example 24: Preparation of 3-(2-methylcinnolin-2-ium-4-yl)propanoic Acid Chloride A55

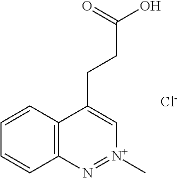 [SMILES: C[n+]1cc(CCC(=O)O)c2ccccc2n1 ; [Cl-]]

### Step 1: Preparation of methyl 3-cinnolin-4-ylpropanoate

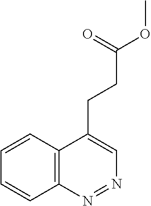 [SMILES: COC(=O)CCc1cnnc2ccccc12]

A microwave vial was charged with 4-chlorocinnoline (0.5 g), methyl acrylate (0.547 mL), palladium (II) acetate (0.034 g), tris-o-tolylphosphane (0.102 g), triethylamine (1.27 mL) and N,N-dimethylformamide (9.87 mL), purged with nitrogen then heated at 150° C. under microwave irradiation for 30 minutes. The reaction mixture was diluted with dichloromethane and washed with water (3×). The organic phase was concentrated, then purified by silica gel chromatography eluting with 0 to 10% methanol in dichloromethane to give methyl 3-cinnolin-4-ylpropanoate as a brown gum.

¹H NMR (400 MHz, CDCl₃) 9.20 (s, 1H), 8.56 (d, 1H), 8.08-8.00 (m, 1H), 7.90-7.76 (m, 2H), 3.71 (s, 3H), 3.43 (t, 2H), 2.82 (t, 2H)

### Step 2: Preparation of methyl 3-(2-methylcinnolin-2-ium-4-yl)propanoate Iodide A81

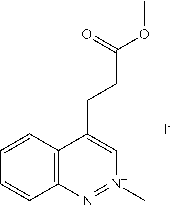 [SMILES: COC(=O)CCc1c[n+](C)nc2ccccc12 ; "l-"]

To a stirred solution of methyl 3-cinnolin-4-ylpropanoate (0.503 g) in acetone (9.89 mL) was added iodomethane (1.23 mL) and lithium chloride (0.008 g). The reaction mixture was heated at 40° C. for 6 hours. The reaction mixture was cooled to room temperature and allowed to stand overnight. The reaction mixture was concentrated to give methyl 3-(2-methylcinnolin-2-ium-4-yl)propanoate iodide which was used without further purification.

¹H NMR (400 MHz, CD₃OD) 9.69 (s, 1H), 8.63-8.55 (m, 2H), 8.38-8.29 (m, 2H), 4.89 (s, 3H), 3.75-3.65 (m, 5H), 3.05-3.00 (m, 2H)

### Step 3: Preparation of 3-(2-methylcinnolin-2-ium-4-yl)propanoic acid 2,2,2-trifluoroacetate A56

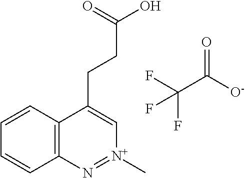 [SMILES: C[n+]1cc(CCC(=O)O)c2ccccc2n1 ; O=C([O-])C(F)(F)F]

A mixture of methyl 3-(2-methylcinnolin-2-ium-4-yl)propanoate iodide (0.723 g) and aqueous 2M hydrochloric acid (16.1 mL) was heated at 60° C. for 2.5 hours. The reaction mixture was cooled to room temperature and allowed to stand for 72 hours. The reaction mixture was concentrated and purified by preparative reverse phase HPLC (trifluoroacetic acid was present in the eluent) to give 3-(2-methylcinnolin-2-ium-4-yl)propanoic acid 2,2,2-trifluoroacetate.

¹H NMR (400 MHz, CD₃OD) 9.66 (s, 1H), 8.64-8.54 (m, 2H), 8.38-8.28 (m, 2H), 4.91 (s, 3H), 3.69 (t, 2H), 2.97 (t, 2H) (CO₂H proton missing)

### Step 4: Preparation of 3-(2-methylcinnolin-2-ium-4-yl)propanoic Acid Chloride A55

A column was packed with Discovery DSC-SCX ion exchange resin (2 g). It was washed with methanol (3 column volumes). To this was added 3-(2-methylcinnolin-2-ium-4-yl)propanoic acid 2,2,2-trifluoroacetate (0.11 g) dissolved in a minimum amount of methanol. The column was eluted with methanol (3 column volumes) and then eluted with 3M hydrogen chloride in methanol (3 column volumes). The methanolic hydrogen chloride fractions were combined and concentrated to give 3-(2-methylcinnolin-2-ium-4-yl)propanoic acid chloride as a green gum.

¹H NMR (400 MHz, CD₃OD) 9.73 (s, 1H), 8.65-8.54 (m, 2H), 8.41-8.28 (m, 2H), 4.93 (s, 3H), 3.78-3.70 (m, 2H), 3.04 (t, 2H) (CO₂H proton missing)

### Example 25: Preparation of (2-ethylcinnolin-2-ium-4-carbonyl)-methylsulfonyl-azanide A22

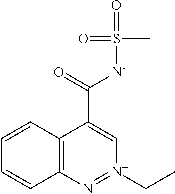 [SMILES: CC[n+]1cc(C(=O)[N-]S(C)(=O)=O)c2ccccc2n1]

A mixture of iodoethane (0.2 mL) and N-methylsulfonylcinnoline-4-carboxamide (0.2 g) in acetonitrile (5 mL) was heated at 80° C. overnight. A further aliquot of iodoethane (0.2 mL) was added and heating continued again overnight. A third aliquot of iodoethane (0.2 mL) was added and heating continued again overnight. The reaction mixture was concentrated and the residue partitioned between dichloromethane and water. The aqueous phase was concentrated and purified by preparative reverse phase HPLC to give (2-ethylcinnolin-2-ium-4-carbonyl)-methylsulfonyl-azanide.

¹H NMR (400 MHz, CD₃OD) 9.99 (s, 1H), 8.98-8.92 (m, 1H), 8.68-8.60 (m, 1H), 8.40-8.32 (m, 2H), 5.19 (q, 2H), 3.34-3.32 (m, 3H), 1.84 (t, 3H)

### Example 26: Preparation of ethyl 2-(2-methylcinnolin-2-ium-4-yl)acetate 2,2,2-trifluoroacetate A52

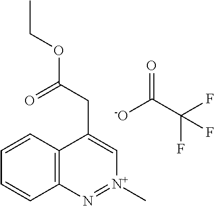

### Step 1: Preparation of diethyl 2-cinnolin-4-ylpropanedioate

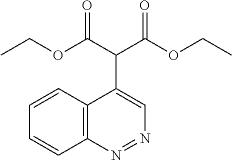 [SMILES: CCOC(=O)C(C(=O)OCC)c1cnnc2ccccc12]

To a suspension of 4-(p-tolylsulfonyl)cinnoline (1 g) and dicesium carbonate (5.74 g) in N,N-dimethylformamide (35.17 mL) was added diethyl propanedioate (0.854 g). The mixture was stirred at room temperature for 72 hours. and the reaction stirred at room temperature over the weekend. The reaction mixture was partitioned between water and dichloromethane. The organic layer was washed with water (5×), concentrated then purified by silica gel chromatography eluting with a mixture of methanol and dichloromethane to give diethyl 2-cinnolin-4-ylpropanedioate.

¹H NMR (400 MHz, CD₃OD) 9.36 (s, 1H), 8.49-8.57 (m, 1H), 8.22 (d, 1H), 7.88-8.05 (m, 2H), 5.49 (s, 1H), 4.27 (dd, 4H), 1.25 (s, 6H)

### Step 2: Preparation of ethyl 2-cinnolin-4-ylacetate

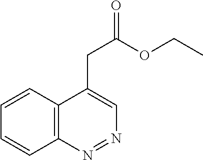 [SMILES: CCOC(=O)Cc1cnnc2ccccc12]

To a solution of diethyl 2-cinnolin-4-ylpropanedioate (0.2 g) in DMSO (6.94 mL) was added a solution of sodium chloride (0.049 g) in water (0.5 mL). The mixture was heated at 150° C. for 3 hours. The mixture was concentrated and purified by silica gel chromatography eluting with a mixture of ethyl acetate and iso-hexane to give ethyl 2-cinnolin-4-ylacetate, which was used without purification in the next step.

### Step 3: Preparation of ethyl 2-(2-methylcinnolin-2-ium-4-yl)acetate 2,2,2-trifluoroacetate A52

A mixture of ethyl 2-cinnolin-4-ylacetate (0.04 g) and iodomethane (0.115 mL) in acetone (1 mL) was stirred at room temperature overnight. The reaction mixture was concentrated then purified by preparative reverse phase HPLC (trifluoroacetic acid was present in the eluent) to give ethyl 2-(2-methylcinnolin-2-ium-4-yl)acetate 2,2,2-trifluoroacetate.

¹H NMR (400 MHz, CD₃OD) 9.72 (s, 1H), 8.66-8.57 (m, 1H), 8.56-8.48 (m, 1H), 8.40-8.31 (m, 2H), 4.92 (s, 3H), 4.23 (q, 2H), 1.28 (t, 3H) (CH₂ exchanged)

### Example 27: Preparation of 3-[2-(3-phosphonopropyl)cinnolin-2-ium-4-yl]propylphosphonic Acid Chloride A82

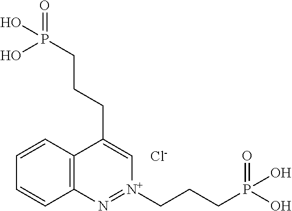 [SMILES: O=P(O)(O)CCCc1c[n+](CCCP(=O)(O)O)nc2ccccc12 ; [Cl-]]

### Step 1: Preparation of 2,4-bis(3-diethoxyphosphorylpropyl)cinnolin-2-ium bromide

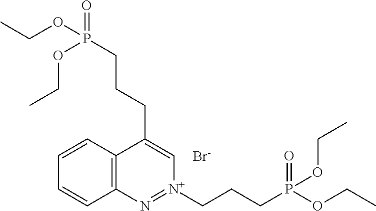 [SMILES: CCOP(=O)(CCCc1c[n+](CCCP(=O)(OCC)OCC)nc2ccccc12)OCC ; [Br-]]

To a solution of 1-bromo-3-diethoxyphosphoryl-propane (1.9 g) in N,N-dimethylformamide (5 mL) was added cinnoline (0.5 g) and sodium iodide (catalytic) at room temperature. The reaction mixture was heated at 100° C. for 4 hours. The reaction mixture was concentrated to afford crude 2,4-bis(3-diethoxyphosphorylpropyl)cinnolin-2-ium bromide as a dark brown liquid, which was used without further purification.

### Step 2: Preparation of 3-[2-(3-phosphonopropyl)cinnolin-2-ium-4-yl]propylphosphonic Acid Chloride A82

A solution of 2,4-bis(3-diethoxyphosphorylpropyl)cinnolin-2-ium bromide (0.75 g) in conc. hydrochloric acid (10 mL) was heated at 100° C. for 16 hours. The reaction mixture was cooled to room temperature, concentrated and purified by preparative reverse phase HPLC 3-[2-(3-phosphonopropyl)cinnolin-2-ium-4-yl]propylphosphonic acid chloride as a pale yellow liquid.

¹H NMR (300 MHz, D₂O) 9.45 (s, 1H), 8.49-8.41 (m, 2H), 8.22-8.20 (m, 2H), 5.07-5.02 (t, 2H), 3.45-3.40 (t, 2H), 2.43-2.32 (m, 2H), 2.06-2.01 (m, 2H), 1.82-1.65 (m, 4H) (POH protons missing)

Additional compounds in Table A were prepared by analogous procedures, from appropriate starting materials.

TABLE A
Physical data for compounds of the invention

| Compound | | |
|---|---|---|
| Number | Structure | ¹H NMR |
| A1 | 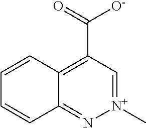 | (400 MHz, CD3OD) 9.84 (s, 1H), 9.14-9.08 (m, 1H), 8.61-8.55 (m, 1H), 8.33 (dqd, 2H), 4.92 (s, 3H) |
| A2 | 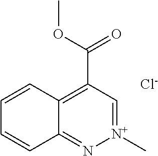 | (400 MHz, CD3OD) 10.16 (s, 1H), 9.24 (d, 1H), 8.70 (d, 1H), 8.49 (ddd, 1H), 8.44-8.37 (m, 1H), 4.99 (s, 3H), 4.19 (s, 3H) |
| A3 | 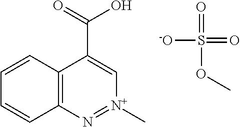 | (400 MHz, CD3OD) 9.65 (d, 1H), 9.20 (d, 1H), 8.64-8.58 (m, 1H), 8.46-8.41 (m, 1H), 8.39-8.31 (m, 1H), 4.94 (s, 3H), 3.66 (s, 3H) (CO2H proton missing) |
| A4 | 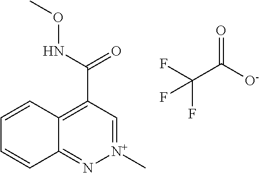 [SMILES: CONC(=O)c1c[n+](C)nc2ccccc12 ; O=C([O-])C(F)(F)F] | (400 MHz, CD3OD) 9.94 (s, 1H), 8.94 (br. s., 1H), 8.65 (d, 1H), 8.49-8.27 (m, 2H), 4.94 (s, 3H), 4.05 (s, 3H) (NH proton missing) |
| A5 | 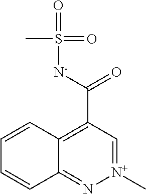 [SMILES: C[n+]1cc(C(=O)[N-]S(C)(=O)=O)c2ccccc2n1] | (400 MHz, CD3OD) 9.87 (s, 1H), 9.24-9.11 (m, 1H), 8.63-8.55 (m, 1H), 8.39-8.26 (m, 2H), 4.84- 4.77 (m, 3H), 3.24 (s, 3H) |
| A6 | 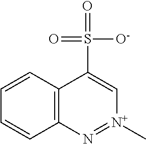 | (400 MHz, D2O) 9.74 (s, 1H), 8.77 (d, 1H), 8.57 (d, 1H), 8.39-3.33 (m, 1H), 8.32-8.25 (m, 1H), 4.85 (s, 3H) |
| A7 | 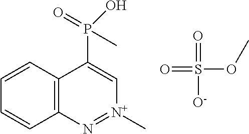 | (400 MHz, CD3OD) 9.80 (d, 1H), 9.02 (d, 1H), 8.71 (d, 1H), 8.53-8.37 (m, 2H), 4.98 (s, 3H), 3.65 (s, 3H), 2.01-1.87 (m, 3H) (POH proton missing) |
| A8 | 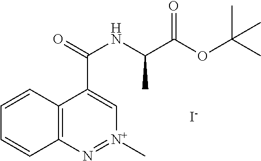 [SMILES: C[C@@H](NC(=O)c1c[n+](C)nc2ccccc12)C(=O)OC(C)(C)C ; [I-]] | (400 MHz, D2O) 9.75 (s, 1H), 8.70-8.64 (m, 1H), 8.60-8.52 (m, 1H), 8.47-8.38 (m, 2H), 4.96 (s, 3H), 4.65 (d, 1H), 1.60-1.50 (m, 12H) (NH proton missing) |
| A9 | 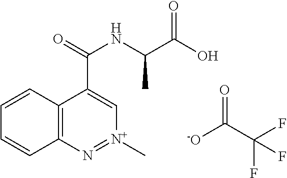 | (400 MHz, D2O) 9.77 (s, 1H), 8.71-8.64 (m, 1H), 8.60-8.50 (m, 1H), 8.48-8.33 (m, 2H), 4.95 (s, 3H), 1.61 (d, 3H) (one CH proton hidden under water peak, NH and CO2H protons missing) |
| A10 | 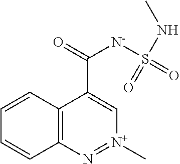 [SMILES: CNS(=O)(=O)[N-]C(=O)c1c[n+](C)nc2ccccc12] | (400 MHz, CD3OD) 10.04 (s, 1H), 8.69 (dd, 2H), 8.56-8.37 (m, 2H), 4.97 (s, 3H), 2.85 (s, 3H) (NH proton missing) |
| A11 | 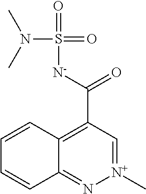 [SMILES: CN(C)S(=O)(=O)[N-]C(=O)c1c[n+](C)nc2ccccc12] | (400 MHz, CD3OD) 10.06 (s, 1H), 8.72 (d, 1H), 8.61 (d, 1H), 8.53-8.38 (m, 2H), 4.98 (s, 3H), 3.11 (s, 6H) |
| A12 | 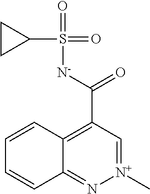 [SMILES: C[n+]1cc(C(=O)[N-]S(=O)(=O)C2CC2)c2ccccc2n1] | (400 MHz, D2O) 9.63 (s, 1H) 8.63 (dd, 1H) 8.44- 8.54 (m, 1H) 8.15-8.32 (m, 2H) 4.83 (s, 3H) 2.90- 3.03 (m, 1H) 0.97-1.23 (m, 4H) |
| A13 | 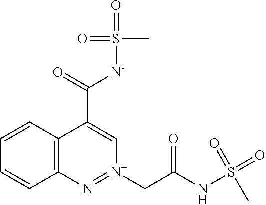 [SMILES: CS(=O)(=O)[N-]C(=O)c1c[n+](CC(=O)NS(C)(=O)=O)nc2ccccc12] | (400 MHz, CD3OD) 10.02-10.16 (m, 1H) 8.76 (t, 2H) 8.39-8.60 (m, 2H) 6.16 (d, 1H) 3.43-3.55 (m, 3H) 3.25-3.37 (m, 3H) (One proton at 6.16 exchanged out) |
| A14 | 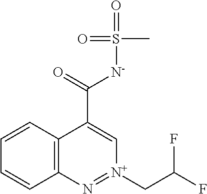 [SMILES: CS(=O)(=O)[N-]C(=O)c1c[n+](CC(F)F)nc2ccccc12] | (400 MHz, CD3OD) 10.16 (s, 1H), 8.80-8.73 (m, 2H), 8.58-8.47 (m, 2H), 6.87-6.56 (m, 1H), 5.68 (dt, 2H), 3.52 (s, 3H) |
| A15 | 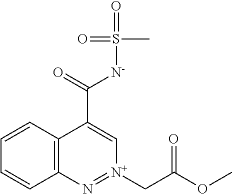 [SMILES: COC(=O)C[n+]1cc(C(=O)[N-]S(C)(=O)=O)c2ccccc2n1] | (400 MHz, CD3OD) 10.01 (s, 1H), 9.27-9.21 (m, 1H), 8.62-8.57 (m, 1H), 8.42-8.31 (m, 2H), 6.11- 6.06 (m, 1H), 3.87 (s, 3H), 3.22 (s, 3H) (One proton at 6.11-6.06 exchanged out) |
| A16 | 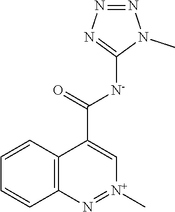 [SMILES: Cn1nnnc1[N-]C(=O)c1c[n+](C)nc2ccccc12] | (400 MHz, CD3OD) 10.20 (s, 1H), 9.13 (d, 1H), 8.72-8.66 (m, 1H), 8.48-8.37 (m, 2H), 5.00 (s, 3H), 4.13-4.10 (m, 3H) |
| A17 | 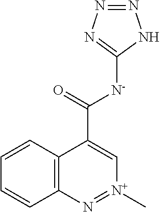 [SMILES: C[n+]1cc(C(=O)[N-]c2nnn[nH]2)c2ccccc2n1] | (400 MHz, D2O) 9.87 (s, 1H), 8.67-8.59 (m, 1H), 8.55 (d, 1H), 8.43-8.20 (m, 2H), 4.91 (s, 3H) (one NH proton missing) |
| A18 | 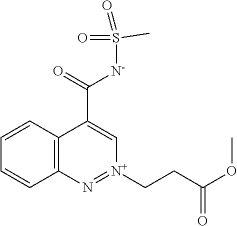 [SMILES: COC(=O)CC[n+]1cc(C(=O)[N-]S(C)(=O)=O)c2ccccc2n1] | (400 MHz, CD3OD) 10.04 (s, 1H), 9.05-8.98 (m, 1H), 8.63-8.54 (m, 1H), 8.40-8.28 (m, 2H), 5.41 (t, 2H), 3.69 (s, 3H), 3.46-3.35 (m, 2H), 3.29 (s, 3H) |
| A19 | 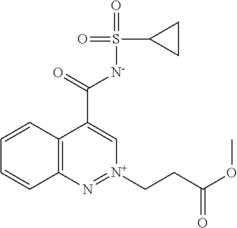 [SMILES: COC(=O)CC[n+]1cc(C(=O)[N-]S(=O)(=O)C2CC2)c2ccccc2n1] | (400 MHz, CD3OD) 10.04 (s, 1H), 9.01-8.95 (m, 1H), 8.64-8.57 (m, 1H), 8.40-8.32 (m, 2H), 5.41 (t, 2H), 3.69 (s, 3H), 3.40 (t, 2H), 3.15-3.08 (m, 1H), 1.32-1.19 (m, 2H), 1.16-1.04 (m, 2H) |
| A20 | 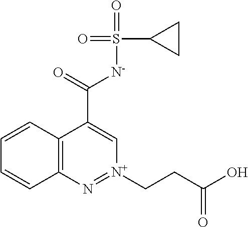 [SMILES: O=C(O)CC[n+]1cc(C(=O)[N-]S(=O)(=O)C2CC2)c2ccccc2n1] | (400 MHz, CD3OD) 10.07 (s, 1H), 8.90-8.84 (m, 1H), 8.67-8.58 (m, 1H), 8.41-8.32 (m, 2H), 5.40 (t, 2H), 3.38 (t, 2H), 3.12 (tt, 1H), 1.30-1.23 (m, 2H), 1.16-1.08 (m, 2H) (CO2H proton missing) |
| A21 | 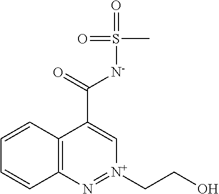 [SMILES: CS(=O)(=O)[N-]C(=O)c1c[n+](CCO)nc2ccccc12] | (400 MHz, CD3OD) 9.95 (s, 1H) 8.89-8.99 (m, 1H) 8.54-8.66 (m, 1H) 8.25-8.40 (m, 2H) 5.18- 5.29 (m, 2H) 4.21-4.31 (m, 2H) 3.28 (s, 3H) (OH proton missing) |
| A22 | 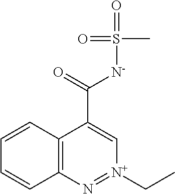 [SMILES: CC[n+]1cc(C(=O)[N-]S(C)(=O)=O)c2ccccc2n1] | (400 MHz, CD3OD) 9.99 (s, 1H), 8.98-8.92 (m, 1H), 8.68-8.60 (m, 1H), 8.40-8.32 (m, 2H), 5.19 (q, 2H), 3.34-3.32 (m, 3H), 1.84 (t, 3H) |
| A23 | 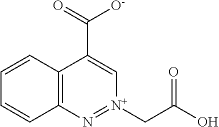 [SMILES: O=C(O)C[n+]1cc(C(=O)[O-])c2ccccc2n1] | (400 MHz, D2O) 9.68 (s, 1H), 8.77-8.73 (m, 1H), 8.53-8.48 (m, 1H), 8.32-8.21 (m, 2H), 5.85 (s, 2H) (CO2H proton missing) |
| A24 | 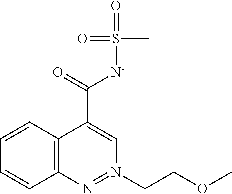 [SMILES: COCC[n+]1cc(C(=O)[N-]S(C)(=O)=O)c2ccccc2n1] | (400 MHz, CD3OD) 9.94 (s, 1H), 9.02-8.95 (m, 1H), 8.65-8.60 (m, 1H), 8.39-8.31 (m, 2H), 5.38- 5.25 (m, 2H), 4.16-4.08 (m, 2H), 3.37-3.33 (m, 3H), 3.32-3.31 (m, 3H) |
| A25 | 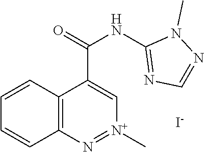 [SMILES: Cn1ncnc1NC(=O)c1c[n+](C)nc2ccccc12 ; [I-]] | (400 MHz, D2O) 9.78 (s, 1H), 8.89-8.78 (m, 1H), 8.57-8.49 (m, 1H), 8.31-8.22 (m, 2H), 8.20 (s, 1H), 4.85 (s, 3H), 3.76 (s, 3H) (NH proton missing) |
| A26 | 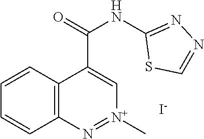 [SMILES: C[n+]1cc(C(=O)Nc2nncs2)c2ccccc2n1 ; [I-]] | (400 MHz, D2O) 9.88 (s, 1H), 9.14 (s, 1H), 8.71- 8.64 (m, 1H), 8.63-8.57 (m, 1H), 8.39-8.27 (m, 2H), 4.90 (s, 3H) (NH proton missing) |
| A27 | 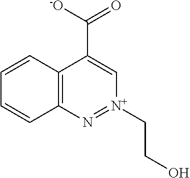 | (400 MHz, CD3OD) 9.94 (s, 1H), 9.22-9.17 (m, 1H), 8.67-8.62 (m, 1H), 8.42-8.31 (m, 2H), 5.25- 5.20 (m, 2H), 4.28-4.21 (m, 2H) (OH proton missing) |
| A28 | 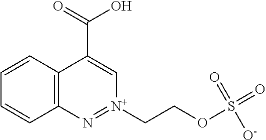 [SMILES: O=C(O)c1c[n+](CCOS(=O)(=O)[O-])nc2ccccc12] | (400 MHz, D2O) 9.67 (s, 1H) 8.64-8.76 (m, 1H) 8.47-8.58 (m, 1H) 8.15-8.33 (m, 2H) 5.29-5.37 (m, 2H) 4.62-4.76 (m, 2H) (CH2 under water peak, CO2H proton missing) |
| A29 | 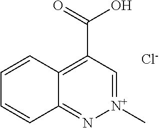 | (400 MHz, D2O) 9.50 (s, 1H), 8.60-8.55 (m, 1H), 8.49-8.45 (m, 1H), 8.26-8.18 (m, 2H), 4.80 (s, 3H) (CO2H proton missing) |
| A30 | 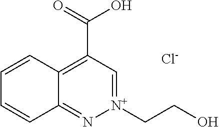 [SMILES: O=C(O)c1c[n+](CCO)nc2ccccc12 ; [Cl-]] | (400 MHz, D2O) 9.66 (s, 1H), 8.75-8.70 (m, 1H), 8.55-8.47 (m, 1H), 8.24 (ddd, 2H), 5.16-5.10 (m, 2H), 4.24-4.18 (m, 2H) (OH and CO2H protons missing) |
| A31 | 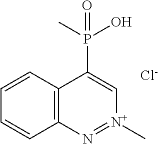 | (400 MHz, CD3OD) 9.82 (d, 1H), 9.02 (d, 1H), 8.72 (d, 1H), 8.48 (dt, 1H), 8.45-8.38 (m, 1H), 4.99 (s, 3H), 1.95 (d, 3H) (POH proton missing) |
| A32 | 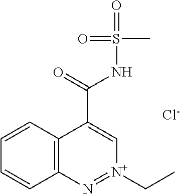 [SMILES: CC[n+]1cc(C(=O)NS(C)(=O)=O)c2ccccc2n1 ; [Cl-]] | (400 MHz, CD3OD) 10.16 (s, 1H), 8.74 (d, 1H), 8.65 (d, 1H), 8.35-8.54 (m, 2H), 5.26 (q, 2H), 3.56 (s, 3H), 1.88 (t, 3H) (NH proton missing) |
| A33 | 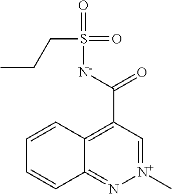 [SMILES: CCCS(=O)(=O)[N-]C(=O)c1c[n+](C)nc2ccccc12] | (400 MHz, CD3OD) 10.03-10.13 (m, 1H) 8.70 (dd, 2H) 8.38-8.54 (m, 2H) 4.93-5.05 (m, 3H) 3.57- 3.73 (m, 2H) 1.90-2.07 (m, 2H) 1.10-1.21 (m, 3H) |
| A34 | 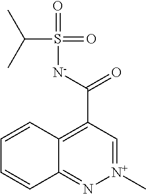 [SMILES: CC(C)S(=O)(=O)[N-]C(=O)c1c[n+](C)nc2ccccc12] | (400 MHz, CD3OD) 9.96-10.13 (m, 1H) 8.69 (dd, 2H) 8.29-8.55 (m, 2H) 4.93-5.07 (m, 3H) 3.98 (spt, 1H) 1.42-1.61 (m, 6H) |
| A35 | 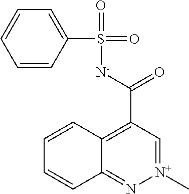 [SMILES: C[n+]1cc(C(=O)[N-]S(=O)(=O)c2ccccc2)c2ccccc2n1] | (400 MHz, CD3OD) 9.97 (s, 1H) 8.62-8.74 (m, 1H) 8.34-8.44 (m, 3H) 8.12-8.24 (m, 2H) 7.61- 7.86 (m, 3H) 4.89-5.02 (m, 3H) |
| A36 | 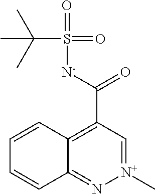 [SMILES: C[n+]1cc(C(=O)[N-]S(=O)(=O)C(C)(C)C)c2ccccc2n1] | (400 MHz, CD3OD) 10.06 (s, 1H) 8.72 (d, 1H) 8.55-8.63 (m, 1H) 8.38-8.52 (m, 2H) 4.99 (s, 3H) 1.51-1.64 (m, 9H) |
| A37 | 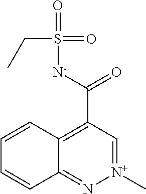 [SMILES: CCS(=O)(=O)[N-]C(=O)c1c[n+](C)nc2ccccc12] | (400 MHz, CD3OD) 10.01-10.12 (m, 1H) 8.61- 8.79 (m, 2H) 8.37-8.53 (m, 2H) 4.99 (s, 3H) 3.61- 3.76 (m, 2H) 1.42-1.57 (m, 3H) |
| A38 | 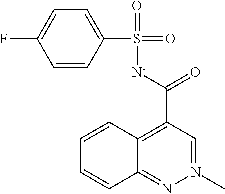 [SMILES: C[n+]1cc(C(=O)[N-]S(=O)(=O)c2ccc(F)cc2)c2ccccc2n1] | (400 MHz, CD3OD) 9.94 (s, 1H), 8.66-8.57 (m, 2H), 8.43-8.32 (m, 2H), 8.25-8.17 (m, 2H), 7.41- 7.33 (m, 2H), 4.93 (s, 3H) |
| A39 | 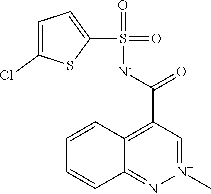 [SMILES: C[n+]1cc(C(=O)[N-]S(=O)(=O)c2ccc(Cl)s2)c2ccccc2n1] | (400 MHz, CD3OD) 9.96 (s, 1H), 8.80-8.70 (m, 1H), 8.68-8.62 (m, 1H), 8.44-8.34 (m, 2H), 7.80- 7.74 (m, 1H), 7.17-7.11 (m, 1H), 4.93 (s, 3H) |
| A40 | 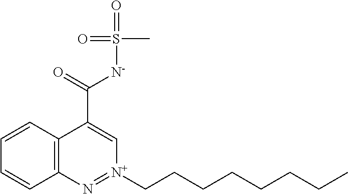 [SMILES: CCCCCCCC[n+]1cc(C(=O)[N-]S(C)(=O)=O)c2ccccc2n1] | (400 MHz, CD3OD) 9.98 (s, 1H), 9.03-8.97 (m, 1H), 8.66-8.60 (m, 1H), 8.40-8.33 (m, 2H), 5.12 (t, 2H), 3.35-3.29 (m, 3H), 2.25 (quin, 2H), 1.53-1.26 (m, 10H), 0.93-0.86 (m, 3H) (SO2Me under solvent peak) |
| A41 | 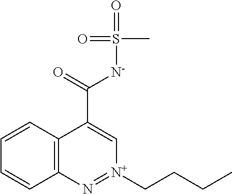 [SMILES: CCCC[n+]1cc(C(=O)[N-]S(C)(=O)=O)c2ccccc2n1] | (400 MHz, CD3OD) 9.99 (s, 1H), 8.99-8.94 (m, 1H), 8.66-8.61 (m, 1H), 8.39-8.33 (m, 2H), 5.13 (t, 2H), 3.34-3.32 (m, 3H), 2.28-2.19 (m, 2H), 1.57- 1.46 (m, 2H), 1.05 (t, 3H) |
| A42 | 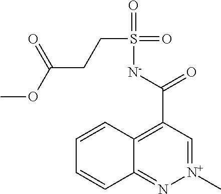 | (400 MHz, CD3OD) 9.91 (s, 1H), 9.06-9.00 (m, 1H), 8.61-8.55 (m, 1H), 8.37-8.27 (m, 2H), 4.94 (s, 3H), 3.75-3.69 (m, 2H), 3.66 (s, 3H), 2.90 (t, 2H) |
| A43 | 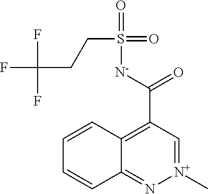 [SMILES: C[n+]1cc(C(=O)[N-]S(=O)(=O)CCC(F)(F)F)c2ccccc2n1] | (400 MHz, CD3OD) 9.90 (s, 1H), 9.24-9.19 (m, 1H), 8.58-8.53 (m, 1H), 8.34-8.27 (m, 2H), 4.91 (s, 3H), 3.61-3.55 (m, 2H), 2.81-2.68 (m, 2H) |
| A44 | 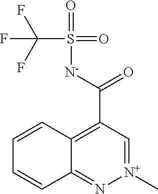 [SMILES: C[n+]1cc(C(=O)[N-]S(=O)(=O)C(F)(F)F)c2ccccc2n1] | (400 MHz, DMSO-d6) 9.98 (s, 1H), 9.04 (d, 1H), 8.64-8.58 (m, 1H), 8.42-8.31 (m, 2H), 4.88 (s, 3H) |
| A45 | 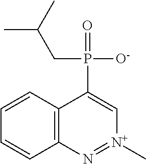 [SMILES: CC(C)CP(=O)([O-])c1c[n+](C)nc2ccccc12] | (400 MHz, D2O) 9.37-9.35 (d, 1H), 8.76-8.74 (d, 1H), 8.48-8.46 (t, 1H), 8.25-8.17 (m, 2H), 4.78 (s, 3H), 1.88-1.76 (m, 3H), 0.83-0.81 (d, 6H) |
| A46 | 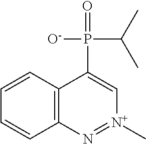 [SMILES: CC(C)P(=O)([O-])c1c[n+](C)nc2ccccc12] | (400 MHz, D2O) 9.38-9.36 (d, 1H), 8.82-8.80 (d, 1H), 8.49-8.47 (d, 1H), 8.25-8.17 (m, 2H), 4.79 (s, 3H), 2.08-2.02 (m, 1H), 0.94-0.88 (m, 6H) |
| A47 | 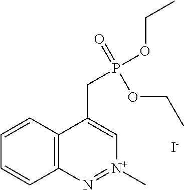 [SMILES: CCOP(=O)(Cc1c[n+](C)nc2ccccc12)OCC ; [I-]] | (400 MHz, CD3OD) 9.69 (d, 1H), 8.68-8.59 (m, 2H), 8.41-8.34 (m, 2H), 4.93 (s, 3H), 4.22-4.10 (m, 4H), 1.27 (t, 6H) (CH2 protons exchanged) |
| A48 | 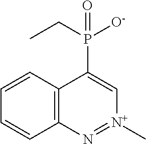 [SMILES: CCP(=O)([O-])c1c[n+](C)nc2ccccc12] | (400 MHz, D2O) 9.38-9.36 (d, 1H), 8.79-8.77 (d, 1H), 8.50-8.47 (d, 1H), 8.27-8.18 (m, 2H), 4.79 (s, 3H), 1.88-1.83 (m, 2H), 0.91-0.82 (m, 3H) |
| A49 | 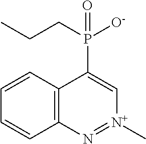 [SMILES: CCCP(=O)([O-])c1c[n+](C)nc2ccccc12] | (300 MHz, D2O) 9.39-9.37 (d, 1H), 8.81-8.79 (d, 1H), 8.52-8.49 (d, 1H), 8.24-8.19 (m, 2H), 4.81 (s, 3H), 1.91-1.81 (m, 2H), 1.39-1.31 (m, 2H), 0.81- 0.79 (m, 3H) |
| A50 | 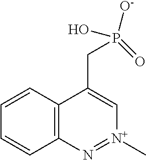 [SMILES: C[n+]1cc(CP(=O)([O-])O)c2ccccc2n1] | (400 MHz, CD3OD) 9.59 (d, 1H), 8.59-8.67 (m, 1H), 8.48-8.57 (m, 1H), 8.25-8.34 (m, 2H), 4.88 (s, 3H), 3.91 (d, 1H) (partial exchange of CH2 at 3.91, POH proton missing) |
| A51 | 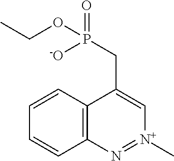 | (400 MHz, CD3OD) 9.57 (d, 1H), 8.60-8.68 (m, 1H), 8.48-8.57 (m, 1H), 8.27-8.35 (m, 2H), 4.89 (s, 3H), 4.00 (quin, 2H), 3.82-3.93 (m, 2H), 1.21 (t, 3H) (Some exchange of CH2 protons) |
| A52 | 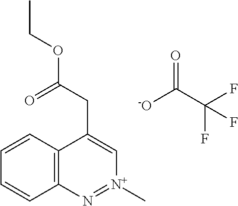 | (400 MHz, CD3OD) 9.72 (s, 1H), 8.66-8.57 (m, 1H), 8.56-8.48 (m, 1H), 8.40-8.31 (m, 2H), 4.92 (s, 3H), 4.23 (q, 2H), 1.28 (t, 3H) (CH2 exchanged) |
| A53 | 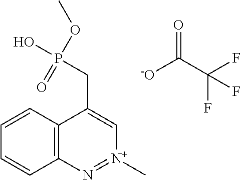 | (400 MHz, CD3OD) 9.55 (d, 1H), 8.66-8.60 (m, 1H), 8.56-8.50 (m, 1H), 8.33-8.27 (m, 2H), 4.88 (s, 3H), 3.91-3.79 (m, 2H), 3.64 (d, 3H) (POH proton missing) |
| A54 | 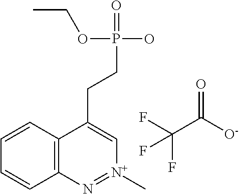 [SMILES: CCOP(=O)(O)CCc1c[n+](C)nc2ccccc12 ; O=C([O-])C(F)(F)F] | (400 MHz, CD3OD) 9.67 (s, 1H), 8.62-8.54 (m, 2H), 8.39-8.30 (m, 2H), 4.90 (s, 3H), 4.11-4.02 (m, 2H), 3.67 (ddd, 2H), 2.36-2.25 (m, 2H), 1.28 (t, 3H) (POH proton missing) |
| A55 | 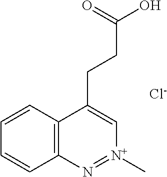 [SMILES: C[n+]1cc(CCC(=O)O)c2ccccc2n1 ; [Cl-]] | (400 MHz, CD3OD) 9.73 (s, 1H), 8.65-8.54 (m, 2H), 8.41-8.28 (m, 2H), 4.93 (s, 3H), 3.78-3.70 (m, 2H), 3.04 (t, 2H) (CO2H proton missing) |
| A56 | 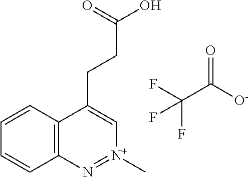 [SMILES: C[n+]1cc(CCC(=O)O)c2ccccc2n1 ; O=C([O-])C(F)(F)F] | (400 MHz, CD3OD) 9.66 (s, 1H), 8.64-8.54 (m, 2H), 8.38-8.28 (m, 2H), 4.91 (s, 3H), 3.69 (t, 2H), 2.97 (t, 2H) (CO2H proton missing) |
| A57 | 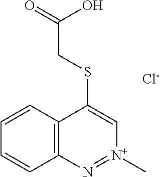 [SMILES: C[n+]1cc(SCC(=O)O)c2ccccc2n1 ; [Cl-]] | (400 MHz, CD3OD) 9.62 (s, 1H), 8.53-8.48 (m, 1H), 8.48-8.42 (m, 1H), 8.34-8.25 (m, 2H), 4.87 (s, 3H), 4.49 (s, 2H) (CO2H proton missing) |
| A58 | 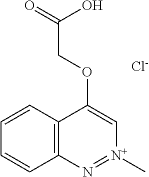 [SMILES: C[n+]1cc(OCC(=O)O)c2ccccc2n1 ; [Cl-]] | (400 MHz, CD3OD) 9.69 (d, 1H), 8.62-8.55 (m, 1H), 8.51 (dd, 1H), 8.34-8.22 (m, 2H), 5.37 (d, 2H), 4.87 (s, 3H) (CO2H proton missing) |
| A59 | 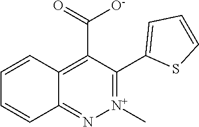 [SMILES: C[n+]1nc2ccccc2c(C(=O)[O-])c1-c1cccs1] | (400 MHz, CD3OD) 8.64-8.58 (m, 1H), 8.37-8.29 (m, 3H), 7.99 (dd, 1H), 7.59 (dd, 1H), 7.37-7.33 (m, 1H), 4.70 (s, 3H) |
| A60 | 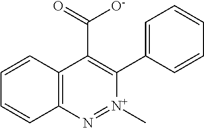 [SMILES: C[n+]1nc2ccccc2c(C(=O)[O-])c1-c1ccccc1] | (400 MHz, CD3OD) 8.66-8.60 (m, 1H), 8.38-8.31 (m, 3H), 7.72-7.65 (m, 5H), 4.60 (s, 3H) |
| A61 | 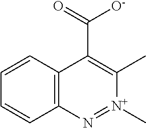 | (400 MHz, CD3OD) 8.38-8.50 (m, 1H), 8.14-8.23 (m, 3H), 4.84 (s, 3H), 2.95-3.13 (m, 3H) |
| A62 | 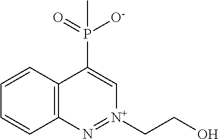 [SMILES: CP(=O)([O-])c1c[n+](CCO)nc2ccccc12] | (400 MHz, D2O) 9.45 (d, 1H), 8.82 (d, 1H), 8.55 (d, 1H), 8.35-8.21 (m, 2H), 5.20-5.11 (m, 2H), 4.26-4.18 (m, 2H), 1.62 (d, 3H) (OH proton missing) |
| A63 | 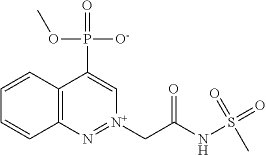 [SMILES: COP(=O)([O-])c1c[n+](CC(=O)NS(C)(=O)=O)nc2ccccc12] | (400 MHz, D2O) 9.46-9.56 (m, 1H) 8.75 (d, 1H) 8.55 (d, 1H) 8.22-8.41 (m, 2H) 5.91-5.99 (m, 2H) 3.51 (s, 3H) 3.17 (s, 3H) (NH proton missing) |
| A64 | 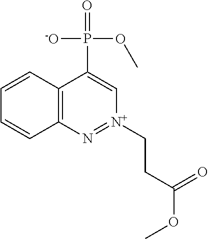 | (400 MHz, CD3OD) 9.79-9.71 (m, 1H), 9.06-8.99 (m, 1H), 8.63-8.54 (m, 1H), 8.39-8.29 (m, 2H), 5.44-5.36 (m, 2H), 3.70-3.66 (m, 3H), 3.66-3.59 (m, 3H), 3.42-3.33 (m, 2H) |
| A65 | 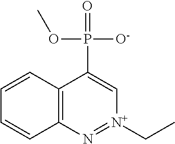 [SMILES: CC[n+]1cc(P(=O)([O-])OC)c2ccccc2n1] | (400 MHz, CD3OD) 9.67 (d, 1H), 9.05-8.99 (m, 1H), 8.66-8.57 (m, 1H), 8.39-8.29 (m, 2H), 5.18 (q, 2H), 3.65-3.60 (m, 3H), 1.84 (t, 3H) |
| A66 | 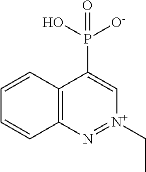 [SMILES: CC[n+]1cc(P(=O)([O-])O)c2ccccc2n1] | (400 MHz, CD3OD) 9.68-9.75 (m, 1H) 9.02-9.09 (m, 1H) 8.56-8.65 (m, 1H) 8.26-8.40 (m, 2H) 5.19 (q, 2H) 1.76-1.89 (m, 3H) (POH proton missing) |
| A67 | 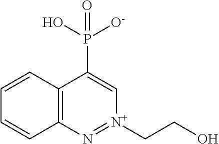 [SMILES: O=P([O-])(O)c1c[n+](CCO)nc2ccccc12] | (400 MHz, D2O) 9.45-9.51 (m, 1H) 8.68-8.74 (m, 1H) 8.47-8.51 (m, 1H) 8.46 (s, 1H) 8.15-8.29 (m, 2H) 5.12 (dd, 2H) 4.14-4.26 (m, 2H) (OH or POH proton missing) |
| A68 | 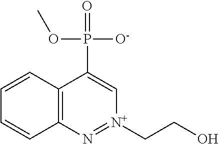 [SMILES: COP(=O)([O-])c1c[n+](CCO)nc2ccccc12] | (400 MHz, D2O) 9.45-9.51 (m, 1H) 8.71 (d, 1H) 8.51-8.57 (m, 1H) 8.20-8.33 (m, 2H) 5.15 (dd, 2H) 4.17-4.25 (m, 2H) 3.47-3.57 (m, 3H) (OH proton missing) |
| A69 | 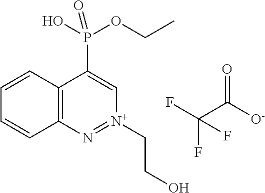 [SMILES: CCOP(=O)(O)c1c[n+](CCO)nc2ccccc12 ; O=C([O-])C(F)(F)F] | (400 MHz, CD3OD) 9.63 (d, 1H), 9.08-9.03 (m, 1H), 8.63 (dd, 1H), 8.40-8.29 (m, 2H), 5.24-5.18 (m, 2H), 4.25-4.20 (m, 2H), 4.02-3.91 (m, 2H), 1.22 (t, 3H) (OH and POH protons missing) |
| A70 | 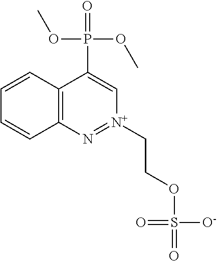 [SMILES: COP(=O)(OC)c1c[n+](CCOS(=O)(=O)[O-])nc2ccccc12] | (400 MHz, CD3OD) 9.94 (d, 1H), 8.80 (s, 2H), 8.54-8.48 (m, 1H), 8.46-8.40 (m, 1H), 5.53-5.41 (m, 2H), 4.70-4.63 (m, 2H), 4.00 (s, 3H), 3.98- 3.95 (m, 3H) |
| A71 | 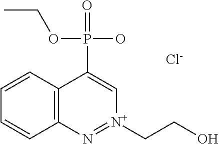 [SMILES: CCOP(=O)(O)c1c[n+](CCO)nc2ccccc12 ; [Cl-]] | (400 MHz, CD3OD) 9.78 (d, 1H), 8.92 (d, 1H), 8.71 (d, 1H0, 8.50-8.36 (m, 2H), 5.31-5.23 (m, 2H), 4.27-4.21 (m, 2H), 4.20-4.10 (m, 2H), 1.31 (t, 3H) (OH and POH protons missing) |
| A72 | 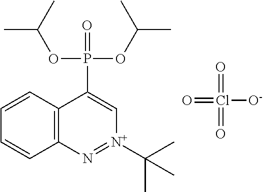 [SMILES: CC(C)OP(=O)(OC(C)C)c1c[n+](C(C)(C)C)nc2ccccc12 ; [O-][Cl+3]([O-])([O-])[O-]] | (400 MHz, D2O) 9.69-9.66 (d, 1H), 8.83-8.81 (d, 1H), 8.65-8.63 (d, 1H), 8.59-8.55 (m, 1H), 8.48- 8.44 (m, 1H), 4.91-4.83 (m, 2H), 1.95 (s, 9H), 1.42-1.41 (d, 6H), 1.24-1.22 (d, 6H) |
| A73 | 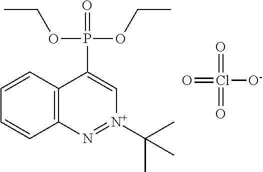 [SMILES: CCOP(=O)(OCC)c1c[n+](C(C)(C)C)nc2ccccc12 ; [O-][Cl+3]([O-])([O-])[O-]] | (400 MHz, D2O) 9.65-9.63 (d, 1H), 8.69-8.67 (d, 1H), 8.61-8.59 (d, 1H), 8.40-8.29 (m, 2H), 4.34- 4.18 (m, 4H), 1.90 (s, 9H), 1.28-1.26 (t, 6H) |
| A74 | 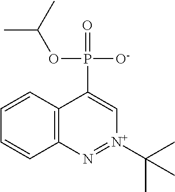 [SMILES: CC(C)OP(=O)([O-])c1c[n+](C(C)(C)C)nc2ccccc12] | (400 MHz, D2O) 9.58-9.56 (d, 1H), 8.68-8.66 (d, 1H), 8.57-8.55 (d, 1H), 8.28-8.19 (m, 2H), 4.43- 4.35 (m, 1H), 1.87 (s, 9H), 1.08-1.06 (d, 6H) |
| A75 | 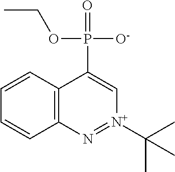 [SMILES: CCOP(=O)([O-])c1c[n+](C(C)(C)C)nc2ccccc12] | (400 MHz, D2O) 9.57-9.55 (d, 1H), 8.69-8.67 (d, 1H), 8.58-8.56 (d, 1H), 8.29-8.20 (m, 2H), 3.85- 3.78 (m, 2H), 1.87 (s, 9H), 1.11-1.07 (t, 3H) |
| A76 | 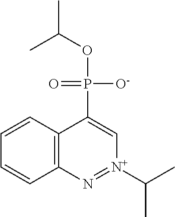 [SMILES: CC(C)OP(=O)([O-])c1c[n+](C(C)C)nc2ccccc12] | (400 MHz, D2O) 9.46-9.43 (d, 1H), 8.70-8.68 (d, 1H), 8.54-8.52 (d, 1H), 8.28-8.19 (m, 2H), 5.46- 5.39 (m, 1H), 4.45-4.37 (m, 1H), 1.72-1.70 (d, 6H), 1.08-1.07 (d, 6H) |
| A77 | 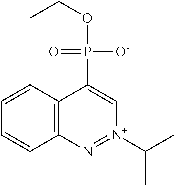 [SMILES: CCOP(=O)([O-])c1c[n+](C(C)C)nc2ccccc12] | (400 MHz, D2O) 9.46-9.44 (d, 1H), 8.70-8.68 (d, 1H), 8.55-8.53 (t, 1H), 8.28-8.20 (m, 2H), 5.46- 5.40 (m, 1H), 3.87-3.80 (m, 2H), 1.72-1.70 (d, 6H), 1.12-1.08 (t, 3H) |
| A78 | 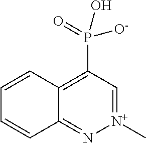 | (400 MHz, D2O) 9.46 (d, 1H), 8.72 (d, 1H), 8.49 (d, 1H), 8.31-8.17 (m, 2H), 4.81 (s, 3H) (POH proton missing) |
| A79 | 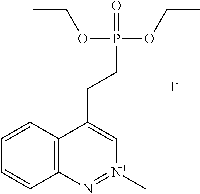 [SMILES: CCOP(=O)(CCc1c[n+](C)nc2ccccc12)OCC ; [I-]] | (400 MHz, CD₃OD) 9.88 (s, 1H), 8.61-8.53 (m, 2H), 8.44-8.32 (m, 2H), 4.96 (s, 3H), 4.28-4.12 (m, 4H), 3.77-3.68 (m, 2H), 2.65-2.53 (m, 2H), 1.37-1.31 (m, 6H) |
| A80 | 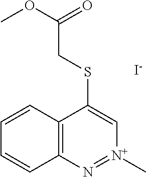 [SMILES: COC(=O)CSc1c[n+](C)nc2ccccc12 ; [I-]] | (400 MHz, CD₃OD) 9.59 (s, 1H), 8.50-8.55 (m, 1H), 8.43-8.48 (m, 1H), 8.26-8.34 (m, 2H), 4.85 (s, 3H), 4.50 (s, 2H), 3.83 (s, 3H) |
| A81 | 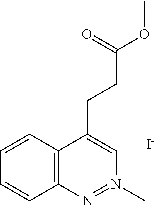 [SMILES: COC(=O)CCc1c[n+](C)nc2ccccc12 ; [I-]] | ¹H NMR (400 MHz, CD₃OD) 9.69 (s, 1H), 8.63- 8.55 (m, 2H), 8.38-8.29 (m, 2H), 4.89 (s, 3H), 3.75-3.65 (m, 5H), 3.05-3.00 (m, 2H) |
| A82 | 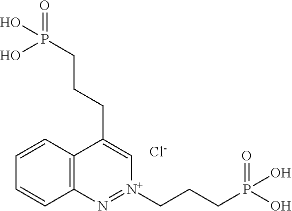 [SMILES: O=P(O)(O)CCCc1c[n+](CCCP(=O)(O)O)nc2ccccc12 ; [Cl-]] | (300 MHz, D₂O) 9.45 (s, 1H), 8.49-8.41 (m, 2H), 8.22-8.20 (m, 2H), 5.07-5.02 (t, 2H), 3.45- 3.40 (t, 2H), 2.43-2.32 (m, 2H), 2.06-2.01 (m, 2H), 1.82-1.65 (m, 4H) (POH protons missing) |

### BIOLOGICAL EXAMPLES

### Post-Emergence Efficacy

Seeds of a variety of test species were sown in standard soil in pots. After cultivation for 14 days (post-emergence) under controlled conditions in a glasshouse (at 24/16° C., day/night; 14 hours light; 65% humidity), the plants were sprayed with an aqueous spray solution derived from the dissolution of the technical active ingredient formula (I) in a small amount of acetone and a special solvent and emulsifier mixture referred to as IF50 (11.12% Emulsogen EL360™+44.44% N-methylpyrrolidone+44.44% Dowanol DPM glycol ether), to create a 50 g/l solution which was then diluted to required concentration using 0.25% or 1% Empicol ESC70 (Sodium lauryl ether sulphate)+1% ammonium sulphate in water as diluent.

The test plants were then grown in a glasshouse under controlled conditions (at 24/16° C., day/night; 14 hours light; 65% humidity) and watered twice daily. After 13 days the test was evaluated (100=total damage to plant; 0=no damage to plant).

The results are shown in Table B (below). A value of n/a indicates that this combination of weed and test compound was not tested/assessed.

### Test Plants:

Ipomoea hederacea (IPOHE), Euphorbia heterophylla (EPHHL), Chenopodium album (CHEAL), Amaranthus palmeri (AMAPA), Lolium perenne (LOLPE), Digitaria sanguinalis (DIGSA), Eleusine indica (ELEIN), Echinochloa crus-galli (ECHCG), Setaria faberi (SETFA)

TABLE B
Control of weed species by compounds of formula (I) after post-emergence application

| Compound | Application | | | | | | | | | |
|---|---|---|---|---|---|---|---|---|---|---|
| No. | Rate g/Ha | AMAPA | CHEAL | EPHHL | IPOHE | ELEIN | LOLPE | DIGSA | SETFA | ECHCG |
| A1 | 1000 | 100 | 100 | 70 | 70 | 100 | 50 | 90 | 30 | 20 |
| A2 | 1000 | 100 | 70 | 70 | 70 | 70 | 30 | 60 | 60 | 30 |
| A3 | 1000 | 20 | 20 | 30 | 20 | 60 | 20 | 50 | 10 | 30 |
| A4 | 1000 | 10 | 40 | 50 | 20 | 20 | 0 | 0 | 10 | 10 |
| A5 | 1000 | 100 | 90 | 90 | 90 | 90 | 50 | 90 | 70 | 50 |
| A6 | 1000 | 10 | 60 | 60 | 0 | 10 | 10 | 10 | 10 | 10 |
| A7 | 1000 | 100 | 100 | 90 | 70 | 80 | 40 | 100 | 70 | 70 |
| A8 | 1000 | 20 | 30 | 30 | 10 | 10 | 20 | 20 | 10 | 20 |
| A9 | 1000 | 50 | 50 | 50 | 50 | 40 | 10 | 40 | 30 | 20 |
| A10 | 1000 | 80 | 70 | 70 | 30 | 60 | 30 | 70 | 50 | 50 |
| A11 | 1000 | 40 | 60 | 70 | 60 | 30 | 20 | 40 | 50 | 50 |
| A12 | 1000 | 60 | 60 | 70 | 70 | 30 | 40 | 20 | 60 | 30 |
| A13 | 1000 | 0 | 0 | 10 | 10 | 10 | 10 | 10 | 10 | 10 |
| A14 | 1000 | 20 | 20 | 40 | 40 | 30 | 0 | 40 | 30 | 20 |
| A15 | 1000 | 50 | 30 | 30 | 10 | 0 | 10 | 0 | 10 | 20 |
| A16 | 1000 | 50 | 60 | 30 | 40 | 0 | 10 | 0 | 10 | 20 |
| A17 | 1000 | 50 | 30 | 20 | 40 | 20 | 10 | 10 | 10 | 0 |
| A18 | 1000 | 10 | 10 | 10 | 20 | 20 | 0 | 30 | 10 | 20 |
| A19 | 500 | 30 | 60 | 40 | 30 | 50 | 20 | 40 | 0 | 60 |
| A20 | 1000 | 50 | 30 | 20 | 10 | 0 | 0 | 0 | 0 | 10 |
| A21 | 1000 | 90 | 70 | 80 | 80 | 80 | 60 | 70 | 90 | 50 |
| A22 | 1000 | 70 | 70 | 60 | 90 | 70 | 60 | 80 | 70 | 50 |
| A23 | 1000 | 50 | 50 | 20 | 20 | 40 | 10 | 70 | 30 | 30 |
| A24 | 1000 | 70 | 70 | 40 | 60 | 50 | 20 | 40 | 50 | 50 |
| A25 | 1000 | 50 | 50 | 20 | 30 | 10 | 10 | 10 | 20 | 10 |
| A26 | 1000 | 20 | 40 | 20 | 30 | 20 | 10 | 0 | 10 | 20 |
| A27 | 1000 | 100 | 100 | 100 | 100 | 70 | 80 | 80 | 80 | 40 |
| A28 | 1000 | 90 | 70 | 30 | 20 | 40 | 50 | 70 | 40 | 20 |
| A29 | 1000 | 90 | 90 | 80 | 80 | 20 | 40 | 30 | 50 | 40 |
| A30 | 1000 | 100 | 100 | 100 | 100 | 80 | 40 | 90 | 70 | 50 |
| A31 | 1000 | 100 | 80 | 100 | 90 | 100 | 70 | 100 | 90 | 60 |
| A33 | 1000 | 40 | 40 | 80 | 60 | 70 | 40 | 80 | 40 | 20 |
| A34 | 1000 | 20 | 50 | 60 | 50 | 40 | 0 | 30 | 60 | 20 |
| A35 | 1000 | 20 | 0 | 40 | 20 | 0 | 0 | 10 | 10 | 0 |
| A36 | 1000 | 40 | 30 | 20 | 30 | 30 | 0 | 10 | 30 | 10 |
| A37 | 1000 | 100 | 80 | 90 | 80 | 70 | 40 | 80 | 60 | 40 |
| A38 | 1000 | 60 | 50 | 60 | 20 | 90 | 0 | 30 | 40 | 20 |
| A39 | 1000 | 80 | 30 | 20 | 30 | 10 | 0 | 10 | 30 | 20 |
| A40 | 1000 | 90 | 70 | 30 | 10 | 10 | 20 | 20 | 40 | 20 |
| A41 | 1000 | 80 | 60 | 100 | 80 | 30 | 30 | 40 | 40 | 20 |
| A42 | 1000 | 70 | 60 | 60 | 40 | 80 | 10 | 60 | 40 | 20 |
| A43 | 1000 | 60 | 30 | 90 | 80 | 90 | 20 | 70 | 50 | 30 |
| A44 | 1000 | 80 | 40 | 30 | 20 | 0 | 0 | 0 | 20 | 20 |
| A45 | 1000 | 100 | 90 | 100 | 90 | 100 | 50 | 90 | 90 | 70 |
| A46 | 1000 | 100 | 100 | 100 | 100 | 100 | 60 | 100 | 90 | 90 |
| A47 | 1000 | 60 | 70 | 30 | 20 | 50 | 10 | 40 | 40 | 30 |
| A48 | 1000 | 100 | 100 | 100 | 70 | 100 | 40 | 70 | 100 | 80 |
| A49 | 1000 | 100 | 90 | 100 | 90 | 100 | 30 | 100 | 90 | 70 |
| A50 | 1000 | 60 | 30 | 20 | 20 | 30 | 0 | 20 | 10 | 20 |
| A51 | 1000 | 90 | 60 | 90 | 60 | 80 | 20 | 90 | 90 | 70 |
| A52 | 500 | 50 | 60 | 40 | 20 | 70 | 10 | 30 | 10 | 10 |
| A53 | 1000 | 100 | 70 | 100 | 100 | 100 | 30 | 90 | 90 | 80 |
| A54 | 500 | 30 | 20 | 80 | 20 | 30 | 20 | 40 | 30 | 30 |
| A55 | 1000 | 90 | 100 | 70 | 70 | 100 | 20 | 100 | 30 | 90 |
| A56 | 500 | 100 | 90 | 40 | 30 | 70 | 10 | 100 | 60 | 70 |
| A57 | 1000 | 40 | 40 | 20 | 10 | 40 | 10 | 30 | 30 | 10 |
| A58 | 1000 | 80 | 70 | 20 | 20 | 30 | 0 | 70 | 60 | 10 |
| A60 | 1000 | 100 | 100 | 100 | 100 | 100 | 40 | 90 | 70 | 50 |
| A61 | 1000 | 100 | 100 | 70 | 100 | 90 | 10 | 70 | 40 | 50 |
| A63 | 1000 | 30 | 30 | 70 | 30 | 50 | 10 | 70 | 60 | 50 |
| A64 | 1000 | 20 | 20 | 70 | 70 | 50 | 10 | 50 | 50 | 50 |
| A65 | 1000 | 90 | 90 | 100 | 70 | 80 | 40 | 90 | 80 | 50 |
| A66 | 1000 | 40 | 30 | 80 | 60 | 70 | 10 | 90 | 70 | 60 |
| A67 | 1000 | 30 | 60 | 30 | 30 | 10 | 0 | 90 | 50 | 20 |
| A68 | 1000 | 100 | 100 | 100 | 100 | 100 | 40 | 100 | 90 | 60 |
| A69 | 500 | 100 | 90 | 100 | 90 | 90 | 40 | 100 | 90 | 70 |
| A70 | 1000 | 50 | 60 | 60 | 30 | 80 | 10 | 70 | 20 | 70 |
| A71 | 1000 | 100 | 90 | 100 | 80 | 90 | 40 | 90 | 100 | 30 |
| A74 | 500 | 100 | 60 | 50 | 50 | 60 | 30 | 20 | 20 | 10 |
| A75 | 500 | 50 | 50 | 30 | 30 | 70 | 30 | 30 | 30 | 70 |
| A76 | 500 | 100 | 90 | 100 | 90 | 100 | 50 | 80 | 80 | 70 |
| A77 | 500 | 100 | 90 | 80 | 90 | 100 | 30 | 90 | 90 | 50 |
| A78 | 1000 | 30 | 10 | 70 | 10 | 40 | 10 | 50 | 40 | 50 |
| A82 | 1000 | 30 | 0 | 20 | 10 | 10 | 10 | 10 | 0 | 20 |

1. A compound of formula (I) or an agronomically acceptable salt or zwitterionic species thereof: 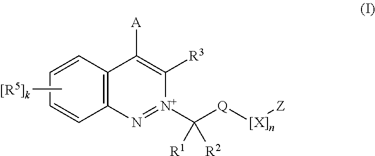
wherein
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O;
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂—NHR⁷, —NHR¹⁵a, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³ is selected from the group consisting of hydrogen, halogen, cyano, nitro, —S(O)ᵣR¹⁵, C₁-C₆alkyl, C₁-C₆halooalkyl, C₁-C₆haloalkoxy, C₁-C₆alkoxy, C₃-C₆cycloalkyl, —N(R⁶)₂, phenyl, a 5- or 6-membered heteroaryl comprising 1, 2, 3 or 4 heteroatoms individually selected from N, O and S, and a 4- to 6-membered heterocyclyl comprising 1, 2 or 3 heteroatoms individually selected from N, O and S, and wherein said phenyl, heteroaryl or heterocyclyl moieties are optionally substituted by 1 or 2 R⁹ substituents;
A is selected from the group consisting of: —C(O)OR⁴¹⁰, —CHO, —C(O)R⁴²⁴, —C(O)NHOR⁴¹¹, —C(O)NHCN, —C(O)NHR⁴²⁵, —S(O)₂NHR⁴²⁵, —C(O)NHS(O)₂R⁴¹⁴, —C(O)NR⁴⁶(CR⁴⁶₂)_{q}—C(O)OR⁴¹⁰, —C(O)NR⁴⁶(CR⁴⁶₂)_{q}S(O)₂OR⁴¹⁰, —C(O)NR⁴⁶(CR⁴⁶₂)_{q}P(O)(R⁴¹³)OR⁴¹⁰, —C(O)NR⁴⁶S(O)₂(CR⁴⁶₂)_{q}C(O)OR⁴¹⁰—(CR⁴⁶₂)_{q}C(O)OR⁴¹⁰, —(CR⁴⁶₂)_{q}S(O)₂OR⁴¹⁰, —(CR⁴⁶₂)_{q}P(O)(R⁴¹³)(OR⁴10), —OC(O)NHOR⁴¹¹—O(CR⁴⁶₂)_{q}C(O)OR⁴¹⁰, —OC(O)NHCN, —O(CR⁴⁶₂)_{q}S(O)₂OR⁴¹⁰—O(CR⁴⁶₂)_{q}P(O)(R⁴¹³)(OR⁴¹⁰), —NR⁴⁶C(O)NHOR⁴¹¹, —NR⁴⁶C(O)NHCN, —C(O)NHS(O)₂R⁴¹², —OC(O)NHS(O)₂R⁴¹², —NR⁴⁶C(O)NHS(O)₂R⁴¹², —S(O)₂OR⁴¹⁰, —OS(O)₂OR⁴¹⁰, —NR⁴⁶S(O)₂OR⁴¹⁰, —NR⁴⁶S(O)OR⁴¹⁰, —NHS(O)₂R⁴¹⁴, —S(O)OR⁴¹⁰, —S(CR⁴⁶₂)_{q}C(O)OR⁴¹⁰, —S(CR⁴⁶₂)_{q}S(O)₂OR⁴¹⁰, —S(CR⁴⁶₂)_{q}P(O)(R⁴¹³)(OR⁴¹⁰), —OS(O)₂OR⁴¹⁰—S(O)₂NHCN, —S(O)₂NHC(O)R⁴¹⁸, —S(O)₂NHS(O)₂R⁴¹², —OS(O)₂NHCN, —OS(O)₂NHS(O)₂R⁴¹², —OS(O)₂NHC(O)R⁴¹⁸, —NR⁴⁶S(O)₂NHCN, —NR⁴⁶S(O)₂NHC(O)R⁴¹⁸, —N(OH)C(O)R⁴¹⁵—ONHC(O)R⁴¹⁵—NR⁴⁶S(O)₂NHS(O)₂R⁴¹², —P(O)(R⁴¹³)(OR⁴¹⁰), —P(O)H(OR⁴¹⁰), —OP(O)(R⁴¹³)(OR⁴10), —NR⁴⁶P(O)(R⁴¹³)(OR⁴10) and tetrazole;
each R⁴⁶ is independently selected from hydrogen and C₁-C₆alkyl;
each R⁴⁹ is independently selected from the group consisting of halogen, cyano, —OH, —N(R⁴⁶)₂, C₁-C₄alkyl, C₁-C₄alkoxy, C₁-C₄haloalkyl and C₁-C₄haloalkoxy;
R⁴¹⁰ is selected from the group consisting of hydrogen, C₁-C₆alkyl, phenyl and benzyl, and wherein said phenyl or benzyl are optionally substituted by 1, 2 or 3 R⁴⁹ substituents, which may be the same or different;
R⁴¹¹ is selected from the group consisting of hydrogen, C₁-C₆alkyl, —C(O)OR⁴¹⁰, and phenyl, and wherein said phenyl is optionally substituted by 1, 2 or 3 R⁴⁹ substituents, which may be the same or different;
R⁴¹² is selected from the group consisting of C₁-C₆alkyl, C₃-C₆cycloalkyl, C₁-C₆haloalkyl, C₁-C₆alkoxy, —OH, —N(R⁴⁶)₂, phenyl, a 5- or 6-membered heteroaryl comprising 1, 2, 3 or 4 heteroatoms individually selected from N, O and S, and a 4- to 6-membered heterocyclyl comprising 1, 2 or 3 heteroatoms individually selected from N, O and S, and wherein said phenyl, heteroaryl or heterocyclyl moieties are optionally substituted by 1 or 2 R⁴²⁰ substituents;
R⁴¹³ is selected from the group consisting of —OH, C₁-C₆alkyl, C₁-C₆alkoxy and phenyl;
R⁴¹⁴ is selected from the group consisting of C₁-C₆alkyl, C₁-C₆haloalkyl, and N(R⁴⁶)₂;
R⁴¹⁵ is selected from the group consisting of C₁-C₆alkyl and phenyl, and wherein said phenyl is optionally substituted by 1, 2 or 3 R⁴⁹ substituents, which may be the same or different;
R⁴¹⁸ is selected from the group consisting of hydrogen, C₁-C₆alkyl, C₁-C₆haloalkyl, C₁-C₆alkoxy, —N(R⁴⁶)₂ and phenyl, and wherein said phenyl is optionally substituted by 1, 2 or 3 R⁴⁹ substituents, which may be the same or different;
each R⁴²⁰ is independently C₁-C₆ alkyl, C₁-C₆alkoxy, halogen, C₁-C₆haloalkyl, C₁-C₆haloalkoxy, or C₁-C₃alkoxyC₁-C₃alkyl;
R⁴²⁴ is a peptide moiety comprising 1, 2, or 3 amino acid moieties, each amino acid moiety independently selected from the group consisting of Ala, Cys, Asp, Glu, Phe, Gly, His, Ile, Lys, Leu, Met, Asn, Pro, Gln, Arg, Ser, Thr, Val, Trp and Tyr, wherein said peptide moiety is bonded to the rest of the molecule via a nitrogen atom in the amino acid moiety;
R⁴²⁵ is phenyl optionally substituted by 1 or 2 R⁴⁹ substituents, or a 5- or 6-membered heteroaryl comprising 1, 2, 3 or 4 heteroatoms individually selected from N, O and S and optionally substituted by 1 or 2 R⁴⁹ substituents;
q is an integer of 1, 2, or 3;
each R⁵ is independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;
k is an integer of 0, 1, 2, 3, or 4;
each R⁶ is independently selected from hydrogen and C₁-C₆alkyl;
each R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, and wherein the aforementioned CR¹R², Q and Z moieties may be attached at any position of said cycloalkyl, phenyl, heteroaryl or heterocyclyl moieties;
n is 0 or 1;
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
Z is selected from the group consisting of hydrogen, methoxy, —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NRS(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl and phenyl, and wherein said phenyl is optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and,
r is 0, 1 or 2,
with the proviso that:
(i) when A is —P(O)(OH)(OR⁴¹⁰) and R⁴¹⁰ is C₁-C₆alkyl, and R¹ and R² are both hydrogen, m is 0, and n is 0, then Z is not hydrogen, and
(ii) the compound of formula (I) is not methyl 2,3-dimethylcinnolin-2-ium-4-carboxylate.

2. The compound according to claim 1, wherein k is 1 or 2, and each R⁵ is is independently selected from the group consisting of halogen, —OR⁷, C₁-C₃alkyl, C₁-C₃haloalkyl, C₃-C₆cycloalkyl, C₁-C₃haloalkoxy, C₂-C₄alkenyl, C₂-C₄alkynyl, C₁-C₃alkoxycarbonyl, C₁-C₃alkylaminocarbonyl, di-C₁-C₃alkylaminocarbonyl and phenyl, wherein said phenyl is optionally substituted by 1, 2 or 3 R⁹, which may be the same or different.

3. The compound according to claim 1, wherein R³ is selected from the group consisting of hydrogen, halogen and C₁-C₆alkyl, phenyl and thiazole, wherein said phenyl or thiazole is optionally substituted by 1 or 2 R⁹, which may be the same or different.

4. The compound according to claim 1, wherein n is 0.

5. The compound according to claim 1, wherein R¹ is selected from the group consisting of hydrogen, halogen, C₁-C₆alkyl, C₁-C₆fluoroalkyl, —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵.

6. The compound according to claim 1, wherein R² is selected from the group consisting of hydrogen, halogen, C₁-C₆alkyl and C₁-C₆fluoroalkyl.

7. The compound according to claim 1, wherein m is 1, 2, or 3.

8. The compound according to claim 7, wherein each R^{1a} and R^{2b} are independently selected from the group consisting of hydrogen, halogen, C₁-C₆alkyl, C₁-C₆fluoroalkyl, —OH, —NH₂, and NHR⁷.

9. The compound according to claim 1, wherein each R^{1a} is hydrogen.

10. The compound according to claim 1, wherein each R^{2b} is independently selected from the group consisting of hydrogen, chloro, fluoro, methyl, ethyl, n-propyl, n-butyl, n-pentyl or n-hexyl.

11. The compound according to claim 1, wherein m is 0,

12. The compound according to claim 1, wherein Z is selected from the group consisting of hydrogen, —CH₂OH, and —OCH₃.

13. The compound according to claim 12, wherein Z is hydrogen.

14. The compound according to claim 1,
wherein Z is selected from the group consisting of —C(O)OR¹⁰, —CHO, —C(O)NHOR¹¹, —C(O)NHCN, —OC(O)NHOR¹¹, —OC(O)NHCN, —NR⁶C(O)NHOR¹¹, —NR⁶C—(O)NHCN, —C(O)NHS(O)₂R¹², —OC(O)NHS(O)₂R¹², —NR⁶C(O)NHS(O)₂R¹², —S(O)₂OR¹⁰, —OS—(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole.

15. The compound according to claim 14, wherein Z is selected from the group consisting of —C(O)OR¹⁰, —CH₂OH, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰, —P(O)(R¹³)(OR¹⁰) and tetrazole.

16. The compound according to claim 1, wherein A is selected from the group consisting of —C(O)OR⁴¹⁰, —C(O)NHOR⁴¹¹, —C(O)NHR⁴²⁵, —S(O)₂NHR⁴²⁵, —C(O)NHS(O)₂R⁴¹⁴—C(O)NR⁴⁶(CR⁴⁶₂)_{q}C(O)OR⁴¹⁰, —C(O)NR⁴⁶S(O)₂(CR⁴⁶₂)_{q}C(O)OR⁴¹⁰—(CR⁴⁶₂)_{q}C(O)OR⁴¹⁰, —(CR⁴⁶₂)_{q}P(O)(R⁴¹³)(OR⁴¹⁰), —OC(O)NHOR⁴¹¹, —O(CR⁴⁶₂)_{q}C(O)OR⁴¹⁰, —OC(O)NHCN, —O(CR⁴⁶₂)_{q}S(O)₂OR⁴¹⁰, —O(CR⁴⁶₂)_{q}P(O)(R⁴¹³)(OR⁴¹⁰), —S(O)₂OR⁴¹⁰, —S(CR⁴⁶₂)_{q}C(O)OR⁴¹⁰, —S(CR⁴⁶₂)_{q}S(O)₂OR⁴¹⁰—P(O)(R⁴¹³)(OR⁴¹⁰), —P(O)H(OR⁴¹⁰), —OP(O)(R⁴¹³)(OR⁴10) and —NR⁴⁶P(O)(R⁴¹³)(OR⁴¹⁰).

17. The compound according claim 16, wherein A is selected from the group consisting of: —C(O)OR⁴¹⁰, —C(O)NHOR⁴¹¹, —C(O)NHR⁴²⁵, —C(O)NHS(O)₂R⁴¹⁴, —C(O)NR⁴⁶(CR⁴⁶₂)_{q}C(O)OR⁴¹⁰, —C(O)NR⁴⁶S(O)₂(CR⁴⁶₂)_{q}C(O)—OR⁴¹⁰, —(CR⁴⁶₂)_{q}C(O)OR⁴¹⁰, —(CR⁴⁶₂)_{q}P(O)(R⁴¹³)(OR⁴10), —S(O)₂OR⁴¹⁰, —S(CR⁴⁶₂)_{q}C(O)OR⁴¹⁰—O(CR⁴⁶₂)_{q}C(O)OR⁴10, and —P(O)(R⁴¹³)(OR⁴¹⁰).

18. The compound according to claim 14, wherein A is selected from the group consisting of —C(O)OR⁴¹⁰, —C(O)NHS(O)₂R⁴¹⁴, —S(O)₂—OR¹⁰, and —P(O)(R⁴¹³)(OR⁴¹⁰).

19. An agrochemical composition comprising a herbicidally effective amount of a compound of formula (I) as defined in claim 1 and an agrochemically-acceptable diluent or carrier.

20. A method of controlling unwanted plant growth, comprising applying a compound of formula (I) as defined in claim 1 to the unwanted plants or to the locus thereof.
